(19) United States
(12) Patent Application Publication
(10) Pub. No.: US 2024/0016900 A1
(43) Pub. Date: Jan. 18, 2024

(54) COLLAGEN PEPTIDE-BASED MEDICAMENT COMPOSITIONS AND USES THEREOF

(71) Applicant: SUSTAIN HOLDINGS, LLC, Stuart, FL (US)

(72) Inventors: Robert O. BARATTA, Palm City, FL (US); Richard E. SCHLUMPF, Stuart, FL (US); Brian J. DEL BUONO, Leesburg, VA (US); David J. CALKINS, Nashville, TN (US)

(21) Appl. No.: 18/342,492

(22) Filed: Jun. 27, 2023

Related U.S. Application Data
(60) Provisional application No. 63/367,080, filed on Jun. 27, 2022.

(51) Int. Cl. A61K 38/39; A61P 27/10; A61K 47/64

(52) U.S. Cl. CPC A61K 38/39; A61P 27/10; A61K 47/6435

(57) ABSTRACT

The present invention is in the fields of medicinal chemistry, biotechnology and pharmaceuticals. The invention provides compositions comprising one or more collagen mimetic peptides, optionally attached to one or more therapeutic compounds or one or more imaging compounds, methods for use of such compositions in treating, preventing, ameliorating, curing and/or diagnosing certain diseases and physical disorders in humans and veterinary animals, including anterior segment ocular diseases and physical disorders, including corneoscleral diseases, disorders or conditions such as myopia, presbyopia, keratoconus and the like. The invention also provides the use of such compositions in treating, preventing, ameliorating, curing and/or diagnosing diseases, disorders and conditions in a variety of other tissues, organs and organ systems. The invention also provides methods of manufacturing such collagen mimetic peptides and compositions. The invention also provides medical devices comprising one or more such compositions of the invention.

### CROSS-REFERENCE TO RELATED APPLICATIONS AND INCORPORATION BY REFERENCE

The present application claims the benefit of the filing date of U.S. Provisional Patent Application No. 63/367,080, filed Jun. 27, 2022, entitled “Collagen Peptide-Based Medicament Compositions and Uses Thereof” and naming as inventors Robert O. Baratta, Richard E. Schlumpf, Brian J. Del Buono and David J. Calkins, and the entirety of which is incorporated herein by reference.

### STATEMENT REGARDING FEDERALLY SPONSORED RESEARCH AND DEVELOPMENT

Not applicable.

### NAMES OF THE PARTIES TO A JOINT RESEARCH AGREEMENT

Not applicable.

### REFERENCE TO SEQUENCE LISTING SUBMITTED ELECTRONICALLY

The contents of the electronic sequence listing (0123-0013US1_SL.xml; Size: 1,851,478 bytes; and Date of Creation Sep. 25, 2023) submitted herewith, is herein incorporated by reference in its entirety.

### BACKGROUND OF THE INVENTION

### Field of the Invention

The present invention is in the fields of medicinal chemistry, biotechnology, pharmaceuticals and medical devices, as well as the use of medicinal compounds and medical devices for the treatment, prevention and amelioration of diseases, disorders and physical ailments in humans and veterinary animals, particularly certain ocular diseases and disorders including corneoscleral diseases and disorders such as myopia, keratoconus, scleritis, episcleritis and presbyopia.

### Background Art

Collagen is the most abundant protein in vertebrates, and is the fundamental structural protein for vertebrate tissues, occurring in virtually every tissue including skin and other epithelial tissues (including the lining of most luminal organs such as those of the gastrointestinal tract), tendons, bone, blood vessels, cartilage, ligaments and teeth. In humans, collagen makes up about a third of the total protein and about three-quarters of the dry weight of skin (see Shoulders, M. D., and Raines, R. T., Ann. Rev. Biochem. 78:929-958 (2009); Gelse, K., et al., Adv. Drug Deliv. Rev. 55:1531-1546 (2003)).

Collagen is a fibrous protein that is composed of a triple helix, which generally consists of two identical chains and a third chain that differs slightly in its chemical composition. Mammals produce at least 46 distinct collagen polypeptide chains that combine to form variants or “types” of collagen. To date, 28 types of collagen have been described. Collagen types are generally grouped according to their structural forms: fibrillar (types I, II, III, V and XI) which represent about 90% of all collagen protein found in mammals, and non-fibrillar (basement membrane or type IV, and other non-fibrillar collagen types with interrupted helix structures) see Id.). The five most common types of collagen, and their tissue distributions, are:

Type I: skin, tendon, organs, bone, vascular connective tissue;

Type II: cartilage;

Type III: reticular connective tissue, often associated with Type I collagen;

Type IV: basement membranes of epithelial tissues and certain solid tumors; and

Type V: hair, placenta, external cellular membranes.

In each of these variants, the polypeptide chains of collagen are composed of approximately 300 repeats of the amino acids proline (Pro), 4(R)-hydroxyproline (Hyp) and glycine (Gly), usually in the sequence X-Y-Gly, where X is often a Pro residue and Y is often a (Hyp) residue; in vertebrates, the typical repeat motif in collagen is ProProGly (see Hulmes, D. J. S., “Collagen Diversity, Synthesis and Assembly,” in: Collagen: Structure and Mechanics, P. Fratzl, Ed., New York: Springer, pp. 15-47 (2008)). Subsequently, in vivo, the hydroxylation of Pro residues is performed enzymatically after collagen biosynthesis but before the chains begin to form a triple helix. Thus, hydroxylation of at least one Pro residue in the ProProGly motif, typically forming ProHypGly, appears to be important for both the proper folding and stability of the collagen triple helix, both of which are key to the normal structure and function of collagen in vivo (see Shoulders, M. D., and Raines, R. T., Ann. Rev. Biochem. 78:929-958 (2009)). For example, the melting temperature of a triple helix of (ProHypGly)₁₀ (SEQ ID NO: 396) chains is 58° C., while that of a triple helix of (ProProGly)₁₀ (SEQ ID NO: 479) chains is only 24° C. (Sakakibara et al., Biochim. Biophys. Acta, 303:198-202 (1973)), and the rate at which (ProHypGly)₁₀ (SEQ ID NO: 396) chains fold into a triple helix is substantially greater than the corresponding rate for (ProProGly)₁₀ SEQ ID NO: 479) chains (Chopra and Ananthanarayanan, Proc. Natl. Acad. Sci. USA, 79:7180-7184 (1982)).

Type I collagen is the most abundant and best-studied collagen. In humans and most other animals it forms more than 90% of the organic mass of bone and is the major collagen of tendons, skin, ligaments, cornea, and many interstitial connective tissues with the exception of a very few such as hyaline cartilage, brain and the vitreous body. The collagen type I triple helix is usually formed as a heterotrimer by two identical al chains and one α2 chain. The triple helical fibers are, in vivo, primarily incorporated into composite fibrils containing other types of collagens, which as noted above vary depending upon tissue type and location (Fleischmajer, E. D. et al., J. Struct. Biol. 105: 162-169 (1990); Niyibizi, C. and Eyre, D. R., Connect. Tissue Res. 20: 247-250 (1989)). In most organs and notably in tendons and fascia, type I collagen provides tensile rigidity and in bone, it defines the biomechanical properties relating to load bearing, tensile strength and torsional stiffness.

In connective tissues (such as bone, tendon, cartilage, ligament, skin, blood vessels and teeth), individual collagen molecules are wound together in tight triple helices. These helices are organized into fibrils of great tensile strength (Jones & Miller, J. Mol. Biol., 218:209-219 (1991)) via cross-linking of individual triple helix fibers (Lodish, H. et al., “Collagen: The Fibrous Proteins of the Matrix”, in: Molecular Cell Biology, 4th ed., Section 22.3, New York: W. H. Freeman (2000)). Varying the arrangements and cross linking of the collagen fibrils enables vertebrates to support stress in one dimension (tendons), two dimensions (skin) or three dimensions (cartilage).

Collagens serve within the body to a considerable extent for the maintenance of the structural integrity of tissues and organs. In all parenchymal organs, collagens represent the major component of the interstitial matrix as well as the basement membranes, while in all connective tissues, particularly bone and cartilage, collagens provide the major functional backbone of the structures. Besides the biomechanical aspects, however, collagens are also involved in a variety of additional functions. For example, specific cell surface and intracellular receptors interact with collagens, and signaling by these receptors is involved in cellular adhesion, differentiation, growth and other cellular activities, as well as the survival of cells both in vivo and in vitro (Vogel, W. F., Eur. J. Dermatol. 11: 506-514 (2001); Gelse, K., et al., Adv. Drug Deliv. Rev. 55:1531-1546 (2003)). Collagens also are involved in the entrapment, local storage and delivery of growth factors and cytokines in a variety of tissues in which the collagens are found. Through these receptor interactions and storage and delivery functions, collagen plays a key role in organ development, wound healing and tissue repair (Chattopadhyay, S. and R. Raines, Biopolymers 101: 821-833 (2014); Yamaguchi, Y. et al., Nature 346: 281-284 (1990); Hay, E. D., J. Cell Biol. 91:205s-223s (1981); Bautista, C. M. et al., Metabolism 39: 96-100 (1990); Zhu, Y. et al., J. Cell Biol. 144: 1069-1080 (1998); Schlegel, K. A. et al., Biomaterials 25:5387-5393 (2004); Kumar, V. A., et al., Biomacromol. 15: 1484-1490 (2014)). These functions also qualify collagens as candidate transport vehicles for the delivery of therapeutic compounds (see, e.g., Chattopadhyay, S. et al., J. Tissue Eng. Regen. Med. 10:1012-1020 (2012); Schuppan, D. et al., Gastroenterol. 114: 139-152 (1998); Frenkel, S. R. et al., J. Bone Jt. Surg. 79-B: 831-836 (1997); Albu, M. G. et al., “Collagen-Based Drug Delivery Systems for Tissue Engineering”, in: Biomaterials Applications for Nanomedicine, Pignatello, R. (Ed.), ISBN: 978-953-307-661-4, DOI: 10.5772/22981, Rijeka, Croatia: InTech, available from: https://www.intechopen.com/books/biomaterials-applications-for-nanomedicine/collagen-based-drug-delivery-systems-for-tissue-engineering (2011)), and for use in wound healing by directly promoting tissue repair or regeneration (Wakitani, S. et al., J. Bone Jt. Surg. 71-B: 74-80 (1989); Kumar, V. A., et al., Biomacromol. 15: 1484-1490 (2014)). Collagen (more particularly, disrupted collagen) has also been implicated in tumor progression and metastasis in humans and other vertebrates (for a review of this issue, see Fang, M., et al., Tumor Biol. 35:2871-2882 (2014)).

Beyond intact collagen molecules, however, fragments of collagen may also have potential therapeutic uses, and indeed, may perform in a superior fashion relative to native collagen. For example, non-collagenous fragments of collagens IV, XV and XVIII have been shown to promote the growth of blood vessels and tumor cells, and to influence a variety of other cellular activities (Ortega, N. and Werb, Z., J. Cell Sci. 115: 4201-4214 (2002); Davis, G. E. et al., Am. J. Pathol. 156: 1489-1498 (2000); O'Reilly, M. S. et al., Cell 88: 277-285 (1997)). Analogously, as described in greater detail below, fragments or synthetic collagen mimetic peptides (CMPs) of collagen type I have recently been studied for their utility in treatment of diseases and medical disorders, both as active pharmaceutical ingredients (APIs) in their own right and in the delivery of a skin wound-healing agent (see U.S. Pat. Nos. 5,973,112, 7,122,521, 7,858,741, and U.S. Patent Publ. No. US 2007/0275897 A1, the disclosures of all of which are incorporated herein by reference in their entireties; see also e.g., Chattopadhyay, S. et al., J. Tissue Eng. Regen. Med. 10:1012-1020 (2012); Kumar, V. A. et al., Biomacromolecules 15:1484-1490 (2014)).

Collagen abnormalities are associated with a wide variety of human diseases, including diseases and disorders of the eye such as cataracts and glaucoma (Coudrillier, B., et al., PLoS ONE 10: e0131396 (2015); Huang, W. et al., Med. Sci. Monit. Basic Res. 19: 237-240 (2013); Dua, H. S., et al., Br. J. Ophthalmol. 98: 691-697 (2014)), arthritis, rheumatism, brittle bones, atherosclerosis and cirrhosis. Disruptions in collagen are also associated with certain human and veterinary diseases such as certain cancers (particularly carcinomas of the luminal organs, and certain sarcomas); see, e.g., Lauer, J. L., and Fields, G. B., “Collagen in Cancer”, in The Tumor Microenvironment, New York: Springer, pp. 477-507 (2010). Collagen is also critically important in wound healing and is known to be upregulated in areas of epithelial wounds where healing is taking place (see, e.g., U.S. Pat. Nos. 5,973,112 and 7,122,521, which are incorporated herein by reference in their entireties; see also Chattopadhyay, S., et al., J. Tissue Eng. Regen. Med. 10:1012-1020 (2012); Chattopadhyay, S., et al., Org. Biomol. Chem. 10:5892-5897 (2012); Kumar, V. A., et al., Biomacromol. 15: 1484-1490 (2014)), including in the skin and the cornea of the eye. Indeed, collagen, collagen fragments or certain mimetic peptides of natural collagen have been reported to show promise in treating certain wounds and diseases in humans and animals, particularly skin wounds (see, e.g., U.S. Pat. Nos. 5,973,112, 7,122,521, 7,858,741, and U.S. Patent Publ. No. US 2007/0275897 A1, all of which are incorporated herein by reference in their entireties; see also Kumar, V. A. et al., Biomacromolecules 15:1484-1490 (2014)). It is thought that these collagen fragments or collagen mimetic peptides may specifically target areas of collagen disruption associated with skin wounds by intercalating into the disrupted collagen and reforming the native collagen I triple helix (see, e.g., Chattopadhyay, S., et al., J. Tissue Eng. Regen. Med. 10:1012-1020 (2012); Chattopadhyay, S., et al., Org. Biomol. Chem. 10:5892-5897 (2012)). As a result, there have been attempts made to use collagen as a vehicle for delivering certain drugs, with varying degrees of success (see, e.g., B. An, et al., Adv. Drug Deliv. Rev. 97:69-84 (2016); V. Chak, et al., Intl. J. Pharm. Teaching and Practices 4:811 (2013)). Collagen mimetic peptides have also been used in a topical application to deliver a conjugated therapeutic compound, the neuropeptide known as Substance P, to areas of skin wounds; such CMP-Substance P conjugates have been shown to accelerate wound healing in a mouse skin model (Chattopadhyay, S., et al., J. Tissue Eng. Regen. Med. 10:1012-1020 (2012)). Certain extracellular matrix (ECM) components, including collagens, are also involved in maintaining proper structure and function of the nervous system, particularly the peripheral nervous system, and disruption of or damage to these ECM components often leads to nerve cell disorder and/or death (see, e.g., Koopmans G, Hasse B, Sinis N. The role of collagen in peripheral nerve repair (Chapter 19). International Review of Neurobiology. Volume 87: Academic Press, Elsevier; pp. 363-79 (2009); Gao X, et al., Rev. Neurosci. 24(4):443-53 (2013); Campbell I C et al., J. Biomech. Eng. 136(2):021005 (2014); Vecino E et al., J. Cytol. Histol. S3:007 (2015); Vecino E., and Kwok, J. C. F., “The Extracellular Matrix in the Nervous System: The Good and the Bad Aspects”, in Composition and Function of the Extracellular Matrix in the Human Body, F. Travascio, ed., Intech Open, ISBN 978-953-51-2416-0 (2016), accessed Nov. 8, 2019, at http://dx.oi.org/10.5772/62527).

Treatments for diseases/disorders are expensive, difficult to deliver with specificity, and may have deleterious effects at sites distal to the intended site of action. For example, many medicinal compositions, including antibiotics, small molecule therapeutics (e.g., anti-cancer compounds) and biologics (e.g., monoclonal antibody therapeutics) are administered parenterally in a non-targeted fashion and must diffuse or otherwise find their way to the site of the affliction before they are able to provide their therapeutic benefits. This “shotgun approach” to therapy necessarily requires higher dosing and can result in longer periods of therapy and reduced patient compliance than a therapeutic approach which would deliver therapeutic compounds and compositions in a more targeted fashion which would allow for controlled or programmable release at or near the site of the affliction in a human or veterinary animal. In particular, ocular diseases, disorders and physical conditions, particularly those involving the cornea and/or sclera of the eye (referred to herein as “corneoscleral”), have often proven to be difficult to treat and/or remediate. For example, presbyopia is an ocular disorder that is frequently associated with aging (Lafosse, E. et al., Cont. Lens Ant. Eye 43(2): 103-114 (2020); Balgos, M. J. T. D et al., Taiwan J. Ophthalmol 8:121-140 (2018); Katz, J. A. et al., Clin. Ophthalmol. 15:2167-2178 (2021)) and is often treated via optical correction either mechanically (e.g., via eyeglasses or contact lenses) or via refractive surgery (e.g., conductive keratoplasty, LASIK/LASEK surgery, photorefractive keratectomy, or via lens implants). Myopia is an ocular disorder that is found in all ages including both children (Hou, W. et al., Eye Contact Lens 44(4):248-259 (2018); Lau, J. K. et al., Invest. Ophthalmol. 61(2):22 (2020); Tideman, J. W. L. et al., Acta Ophthalmol. 96:301-309 (2018)) and adults (Kim, H. K. et al., Int. J. Ophthalmol. 14(8):1231-1236 (2021); Wang, B. et al., PLoS ONE 12(4):e0175913 (2017); Pugazhendhi, S. et al., Clin. Ophthalmol. 14:853-873 (2020)) which is treated with corrective lenses and/or pharmaceutically, e.g., with atropine or pirenzepine (Gwiazda, J., Optom. Vis. Sci. 86(6):624-628 (2009)) and other pharmacotherapeutic approaches (Wang, W.-Y. et al, Biomed. Pharmacother. 133:111092 (2021)). Particularly in children and the elderly, patient compliance with such corrective measures is often incomplete due to inconvenience, discomfort or the like, leading to continued, and in some cases worsening, loss of vision.

Previous work from some of the present inventors has demonstrated and/or suggested that certain CMP-containing formulations may be useful in treating certain front-of-eye ocular conditions such as dry eye disease and other corneal diseases, disorders and injuries (see, e.g., U.S. Pat. No. 10,632,168; Baratta, R. O. et al., Baratta et al., Front. Pharmacol. 12:705623 (2021); Baratta, R. O. et al., Surv. Ophthalmol. 67:60-67 (2022)), and certain back-of-eye disorders and conditions such as glaucoma, macular degeneration, optic neuropathy and the like (see, e.g., U.S. Pat. No. 11,389,513; McGrady, N. R. et al., Front. Pharmacol. 12:764709 (2021); Ribeiro, M. et al., Int. J. Mol. Sci 23: 2911 (2022); Ribeiro et al., Int. J. Mol. Sci. 23: 7004 (2022)); the disclosure of each of these references is incorporated herein by reference in its entirety. However, this previous work was largely silent as to the potential impact of CMP-containing formulations in treating and/or preventing other eye afflictions such as corneoscleral disorders, diseases and conditions including but not limited to myopia, presbyopia and keratoconus, most of which require mechanical and/or surgical interventions which are often incomplete, uncomfortable or inconvenient for the patient and which often require adjustment and/or re-intervention as the patient ages.

Thus, there is a need in the art for formulations and methods of use that will overcome many of these shortcomings in traditional treatments for certain ocular diseases and disorders, particularly corneoscleral diseases, disorders and conditions, in humans and veterinary animals. Such formulations and methods of use would allow the use of lower doses of medication and more targeted delivery of the medications to the intended sites of action, as well as reducing the therapeutic problems or delays resulting from patient non-compliance. Finally, there is a need in the art for methods of producing such compositions that will meet the needs of the medical and patient communities in maximizing treatment efficacies while reducing costs.

### BRIEF SUMMARY OF THE INVENTION

The present inventors reasoned that since collagen disruption is associated with a variety of diseases and disorders in humans and other animals, the conjugation of a variety of therapeutic compounds and/or diagnostic compounds to collagen or collagen mimetic peptides would provide an elegant, rapid and reproducible way of overcoming many of the above-referenced limitations in treatment and diagnosis of certain physical diseases and disorders and in drug delivery. Thus, the present invention provides compositions suitable for use in methods of treatment and diagnosis of physical disorders and diseases, particularly corneoscleral diseases, disorders and conditions, and provides drug delivery systems, medical devices and methods of manufacturing the same. Accordingly, the present invention meets the needs in the art as expressed hereinabove.

In one aspect, the invention provides compositions comprising one or more collagen mimetic peptides (CMPs), which in certain embodiments have been conjugated one or more therapeutic compounds and/or one or more diagnostic compounds thereby forming CMP conjugates and compositions. Such CMPs and CMP conjugates, and compositions comprising such CMPs and/or CMP conjugates, are useful in treating, preventing, ameliorating and diagnosing a variety of diseases, disorders and physical conditions in humans and veterinary animals. In certain embodiments of this aspect, the invention provides compositions comprising such CMPs and/or CMP conjugates and one or more pharmaceutically acceptable carriers, excipients or compounding agents, and optionally one or more additional therapeutic or diagnostic agents, to provide therapeutic and diagnostic compositions useful in treating, preventing, ameliorating or diagnosing certain diseases and disorders in humans and veterinary animals.

In another aspect, the invention provides methods of treating, preventing, ameliorating or diagnosing certain diseases and disorders in humans and veterinary animals, by administering the conjugates and/or compositions of the invention to a human or veterinary animal suffering from or predisposed to such diseases or disorders. Diseases and disorders suitably treated, prevented, cured, ameliorated or diagnoses according to this aspect of the invention include ocular diseases or disorders, particularly corneoscleral diseases, disorders and conditions, including but not limited to keratoconus, myopia and presbyopia; skin diseases or disorders; cancers; gastrointestinal diseases or disorders; genitourinary tract diseases or disorders; fibrotic diseases or disorders; cardiovascular diseases or disorders; bone diseases or disorders; and rheumatic diseases or disorders.

In yet another aspect, the invention provides medical devices coated with or comprising one or more of the conjugates or compositions of the invention. In related aspects, the invention provides methods of treating, curing, preventing or ameliorating diseases or disorders in humans or veterinary animals comprising implanting one or more of the medical devices of this aspect of the invention into the human or veterinary animal, under conditions such that the disease or disorder is treated, cured, prevented or ameliorated.

In still other aspects, the invention provides methods of manufacturing the compositions, conjugates and medical devices of the invention.

Other objects, advantages, and features of the present invention will be readily apparent to those of ordinary skill in the art upon review of the description, drawings, examples and claims presented herein.

### BRIEF DESCRIPTION OF THE DRAWINGS

FIG. 1 is a pair of photomicrographs depicting images from atomic force microscopy (“AFM”) in a section of mouse eye sclera in the area near the optic nerve head (the peripapillary sclera). White arrows depict the point of measurement of Young's Modulus, shown in subsequent figures. FIG. 1A: lower-power view; FIG. 1B: higher-power view. White bars in each photomicrograph represents 100 μm.

FIG. 2 is a bar graph showing Young's Modulus measurements (in kilopascals (KPa)) in representative peripapillary scleral locations measured by AFM as described in FIG. 1, after treatment with 50 μg/ml MMP1 for 30 minutes and then no subsequent treatment or treatment with a CMP formulation of the invention (comprising SEQ ID NO:1) for 60 minutes. Each individual dot represents one Young's Modulus measurement in a single location. Baseline: no MMP or CMP treatment. Bar heights represent means of all measurements in that group (ANOVA p<0.0001).

FIG. 3 is a series of bar graphs showing Young's Modulus measurements in representative peripapillary scleral locations as measured by AFM before and after treatment with 50 μg/ml MMP1 for 30 minutes (FIG. 3A) and then treatment with a CMP formulation of the invention (comprising SEQ ID NO:1) for either 30 minutes (FIG. 3B) or 60 minutes (FIG. 3C). Each individual dot represents one Young's Modulus measurement in a single location. Baseline: no MMP or CMP treatment. Bar heights represent means of all measurements in that group, with p values (ANOVA) shown between groups in a given pair.

FIG. 4 is a pair of bar graphs showing Young's Modulus measurements in representative peripapillary scleral locations as measured by AFM before and after treatment with 50 μg/ml MMP1 for 30 minutes (FIG. 4A) and then treatment with a CMP formulation of the invention (comprising SEQ ID NO:1) for 30 minutes (FIG. 4B). Each individual dot represents one Young's Modulus measurement in a single location. Baseline: no MMP or CMP treatment. Bar heights represent means of all measurements in that group, with p values (ANOVA) shown between groups in a given pair.

FIG. 5 is a series of bar graphs showing Young's Modulus measurements in representative peripapillary scleral locations as measured by AFM before and after treatment with 50 μg/ml MMP1 for 30 minutes (FIG. 5A) and then treatment with a CMP formulation of the invention (comprising SEQ ID NO:1) for either 30 minutes (FIG. 5B) or 60 minutes (FIG. 5C). Each individual dot represents one Young's Modulus measurement in a single location. Baseline: no MMP or CMP treatment. Bar heights represent means of all measurements in that group, with p values (ANOVA) shown between groups in a given pair.

FIG. 6 is a pair of bar graphs showing Young's Modulus measurements in representative peripapillary scleral locations as measured by AFM before and after treatment with 50 μg/ml MMP1 for 30 minutes (FIG. 6A) and then treatment with a CMP formulation of the invention (comprising SEQ ID NO:1) for 30 minutes (FIG. 6B). Each individual dot represents one Young's Modulus measurement in a single location. Baseline: no MMP or CMP treatment. Bar heights represent means of all measurements in that group, with p values (ANOVA) shown between groups in a given pair.

FIG. 7 is a scatter plot showing the mean Young's Modulus measurements in representative scleral locations as measured by AFM before and after treatment with 50 μg/ml MMP1 for 30 minutes or 45 minutes, and samples treated for 45 minutes with MMP and then with a CMP formulation of the invention (comprising SEQ ID NO:1) for 30 minutes or 60 minutes. Each point represents the mean of several hundred measurements at a particular peripapillary scleral location, all normalized with respect to its own baseline value (thus baseline means are all 1). Baseline: no MMP or CMP treatment. Error bars show standard error of measurement.

FIG. 8 is a pair of bar graphs showing Young's Modulus measurements in representative peripapillary scleral locations as measured by AFM before and after treatment with 50 μg/ml MMP1 for 30 minutes and then treatment with a CMP formulation of the invention (comprising SEQ ID NO:1, or “CMP3”) for 60 minutes. Baseline: no MMP or CMP treatment; 26 locations, 6656 independent measurements. MMP1: treatment with MMP1 for 30 minutes; 20 locations, 5120 independent measurements. CMP3: treatment with MMP1 for 30 minutes and then with CMP3-containing formulation for 60 minutes; 12 locations, 2938 independent measurements. Bar heights represent means of all measurements in that group. FIG. 8A: mean measurements for each location are shown; each individual dot represents the mean of a number of individual measurements of Young's Modulus measurement in a single location. FIG. 8B: mean measurements shown in FIG. 8A, with individual location means removed and rescaled to show the differences in Young's Modulus measurements between the individual treatment groups. Error bars represent standard deviation across all measurements in that group (ANOVA p<0.001 between groups).

FIG. 9 is a pair of bar graphs showing Young's Modulus measurements in representative peripapillary scleral locations as measured by AFM before and after treatment with 50 μg/ml MMP1 for 45 minutes and then treatment with a CMP formulation of the invention (comprising SEQ ID NO:6, or “CMP13”) for 60 minutes. Baseline: no MMP or CMP treatment; 3 locations, 768 independent measurements. MMP1: treatment with MMP1 for 45 minutes; 3 locations, 768 independent measurements. CMP13: treatment with MMP1 for 45 minutes and then with CMP13-containing formulation for 60 minutes; 3 locations, 768 independent measurements. Bar heights represent means of all measurements in that group. FIG. 9A: mean measurements for each location are shown; each individual dot represents the mean of a number of individual measurements of Young's Modulus measurement in a single location. FIG. 9B: mean measurements shown in FIG. 9A, with individual location means removed and rescaled to show the differences in Young's Modulus measurements between the individual treatment groups. Error bars represent standard deviation across all measurements in that group (ANOVA p<0.001 between groups).

FIG. 10 is a series of photomicrographs depicting images from atomic force microscopy in the glial lamina of a section of mouse eye near the peripapillary sclera. White arrows depict the point of measurement of Young's Modulus, shown in subsequent figures. FIG. 10A: baseline, no MMP or CMP treatment. FIG. 10B: after treatment for 30 minutes with 5 μg/ml MMP1. FIG. 10C: after treatment for 30 minutes with 5 μg/ml MMP1 followed by treatment for 30 minutes with a CMP formulation of the invention (comprising SEQ ID NO:6, or “CMP13”). White bar in FIG. 10A represents 100 μm (same scale for each photomicrograph).

FIG. 11 is a series of bar graphs showing Young's Modulus measurements in representative glial lamina locations as measured by AFM before and after treatment with 5 μg/ml MMP1 for 30 minutes and then treatment with a CMP formulation of the invention (comprising SEQ ID NO:1) or vehicle (PBS) for 30 minutes. Two locations were sampled, with 256 independent measurements made for each treatment condition. Each individual dot represents one Young's Modulus measurement in a single location. Baseline: no MMP or CMP treatment. Bar heights represent means of all measurements in that group, with p values (ANOVA) shown between groups in a given pair. FIG. 11A: Sample 1, baseline vs. MMP treatment vs. MMP+CMP treatment. FIG. 11B: Sample 2, baseline vs. MMP treatment vs. MMP+CMP treatment. FIG. 11C: Sample 2, baseline vs. MMP treatment vs. MMP+PBS treatment.

FIG. 12 is a pair of bar graphs showing Young's Modulus measurements in representative glial locations as measured by AFM before and after treatment with 5 μg/ml MMP1 for 30 minutes and then treatment with a CMP formulation of the invention (comprising SEQ ID NO:1, or “CMP3”) for 60 minutes. Baseline: no MMP or CMP treatment; 43 locations, 13,996 independent measurements. MMP1: treatment with MMP1 for 30 minutes; 9 locations, 2304 independent measurements. CMP3: treatment with MMP1 for 30 minutes and then with CMP3-containing formulation for 60 minutes; 9 locations, 2229 independent measurements. PBS: treatment with MMP1 for 30 minutes and then with PBS for 60 minutes; 4 locations, 1024 independent measurements. Bar heights represent means of all measurements in that group. FIG. 12A: mean measurements for each location are shown; each individual dot represents the mean of a number of individual measurements of Young's Modulus measurement in a single glial lamina location. FIG. 12B: mean measurements shown in FIG. 12A, with individual location means removed and rescaled to show the differences in Young's Modulus measurements between the individual treatment groups. Error bars represent standard deviation across all measurements in that group (ANOVA p<0.001 between groups).

FIG. 13 is a series of photomicrographs showing the microscopic morphology of the glial lamina and peripapillary sclera after treatment with MMP1, with or without subsequent treatment with a CMP formulation of the invention (comprising SEQ ID NO:1). FIG. 13a: representative AFM microscopic view; “A” localizes the glial lamina, “B” localizes the peripapillary sclera. FIGS. 13b-13e: fluorescence microscopic views of the glial lamina (FIGS. 13b and 13c) and peripapillary sclera (FIGS. 13d and 13e), treated only with MMP1 for 30 minutes (FIGS. 13b, 13d), or with MMP1 for 30 minutes and then with the CMP formulation for 30 minutes (FIGS. 13c, 13e). Fluorescent colors represent different components in those areas of the glial lamina and sclera: blue is cell nuclei (DAPI-stained); green is type I collagen (stained with an anti-collagen I antibody (“Col1”)); red shows areas of disrupted or digested collagen (stained with a Cy3-labeled collagen-hybridizing peptide (“RCHP”)).

FIG. 14 is a series of scanning electron micrographs of samples of human type I collagen deposited on a substrate at 10 μg/ml, prior to (FIGS. 14A, 14C) and following (FIGS. 14B, 14D) treatment for 30 minutes with 60 ng/ml MMP1. Magnifications: FIG. 14A=10 Kx; FIG. 14B=8.2 Kx; FIG. 14C=31 Kx; FIG. 14D=16 Kx.

FIG. 15 is a series of AFM micrographs of samples of human type I collagen deposited on a substrate at 10 μg/ml, prior to (FIGS. 15A, 15B) and following (FIGS. 15C, 15D) treatment for 30 minutes with 60 ng/ml MMP1, or for 30 minutes with 100 ng/ml MMP1 and 1 μg/ml of a CMP13 (SEQ ID NO:6)-containing formulation of the invention (FIGS. 15E, 15F). Depictions are through-focus of samples examining the height of the collagen deposited in each treatment condition (FIGS. 15A, 15C, 15E), or the three-dimensional fibrous structure of the collagen on the substrate (FIGS. 15B, 15D, 15F). Scale bars are as shown under each photomicrograph.

### DETAILED DESCRIPTION OF THE INVENTION

Unless defined otherwise, all technical and scientific terms used herein have the same meanings as commonly understood by one of ordinary skill in the arts to which this invention belongs. Although any methods and materials similar or equivalent to those described herein can be used in the practice or testing of the present invention, the preferred methods and materials are described hereinafter.

According to a first aspect, the invention provides compositions suitable for use in a medicament for treating or preventing a disease, disorder, structural abnormality or injury in a human or veterinary animal in need of treatment or prevention of such as a disease, disorder, structural abnormality or injury. In certain embodiments, the compositions provided by the invention comprise (a) at least one collagen mimetic peptide (CMP) attached to at least one additional therapeutic compound (TC) to form a CMP-TC conjugate, and (b) one or more pharmaceutically suitable carriers. In related aspects, the invention provides compositions suitable for use in a diagnostic agent suitable for diagnosing or detecting a disease, disorder, structural abnormality or injury in a human or veterinary animal in need thereof. In certain embodiments, the compositions provided by the invention comprise (a) at least one collagen mimetic peptide (CMP) attached to at least one diagnostic compound or agent (DC) to form a CMP-DC conjugate, and (b) one or more pharmaceutically suitable carriers. In other related embodiments, the compositions provided by the invention comprise (a) at least one collagen mimetic peptide (CMP) and (b) at least one additional therapeutic compound, wherein the CMP and the at least one additional therapeutic compound are admixed in a formulation, or “co-formulated,” optionally together with one or more pharmaceutically suitable carriers. In analogous embodiments, the compositions provided by the invention comprise (a) at least one collagen mimetic peptide (CMP) and (b) at least one diagnostic compound or agent, such as a labeling compound or agent, wherein the CMP and the at least one diagnostic compound or agent are admixed in a formulation, or “co-formulated,” optionally together with one or more pharmaceutically suitable carriers, for use in one or more diagnostic methods of the invention.

In certain embodiments of the invention, the collagen mimetic peptide comprises, consists essentially of or consists of an amino acid sequence that is a multimeric repeat of a specific tripeptide having a sequence (Xaa-Yaa-Gly)ₙ (SEQ ID NO: 417), wherein Xaa is independently selected from the group consisting of proline, 4S-hydroxyproline, fluoroproline, chloroproline, lysine, cysteine and methionine; wherein Yaa is independently selected from the group consisting of proline, 4R-hydroxyproline, fluoroproline, chloroproline, lysine, cysteine and methionine; wherein Gly is a glycine residue; and wherein n is an integer ranging from 1 to 20, such as from 3 to 15, from 5 to 15, or from 5 to 10, and is preferably 5, 6, 7, 8, 9 or 10.

In other embodiments, the invention provides collagen mimetic peptides that comprise one or more alternative amino acids in place of at least one of the amino acids set forth in the tripeptide shown in SEQ ID NO:417, including but not limited to alanine (Ala), glutamine (Gln), glutamic acid (Glu), asparagine (Asn) and aspartic acid (Asp).

In certain embodiments of the invention, the collagen mimetic peptide comprises, consists essentially of or consists of an amino acid sequence that is or corresponds to a 21-mer comprising seven repeats of a three amino acid sequence of proline-proline-glycine ((Pro-Pro-Gly)₇), i.e., an amino acid sequence of: Pro-Pro-Gly-Pro-Pro-Gly-Pro-Pro-Gly-Pro-Pro-Gly-Pro-Pro-Gly-Pro-Pro-Gly-Pro-Pro-Gly (SEQ ID NO:1).

In certain other embodiments of the invention, the collagen mimetic peptide comprises, consists essentially of or consists of an amino acid sequence that is or corresponds to a 21-mer comprising seven repeats of a three amino acid sequence in which hydroxyproline (Hyp), and preferably a 4S-hydroxyproline residue, has been substituted for proline₁ in SEQ ID NO:1, yielding a sequence of seven repeats of 4S-hydroxyproline-proline-glycine ((Hyp-Pro-Gly)₇), i.e., an amino acid sequence of: Hyp-Pro-Gly-Hyp-Pro-Gly-Hyp-Pro-Gly-Hyp-Pro-Gly-Hyp-Pro-Gly-Hyp-Pro-Gly-Hyp-Pro-Gly (SEQ ID NO:2).

In certain other embodiments of the invention, the collagen mimetic peptide comprises, consists essentially of or consists of an amino acid sequence that is or corresponds to a 21-mer comprising seven repeats of a three amino acid sequence in which Hyp, and preferably a 4S-hydroxyproline residue, has been substituted for proline₂ in SEQ ID NO:1, yielding a sequence of seven repeats of 4S-hydroxyproline-proline-glycine ((Pro-Hyp-Gly)₇), i.e., an amino acid sequence of: Pro-Hyp-Gly-Pro-Hyp-Gly-Pro-Hyp-Gly-Pro-Hyp-Gly-Pro-Hyp-Gly-Pro-Hyp-Gly-Pro-Hyp-Gly (SEQ ID NO:3).

In certain other embodiments of the invention, the collagen mimetic peptide comprises, consists essentially of or consists of an amino acid sequence that is or corresponds to a 21-mer comprising seven repeats of a three amino acid sequence in which fluoroproline (Flp) has been substituted for proline₁ in SEQ ID NO:1, yielding a sequence of seven repeats of fluoroproline-proline-glycine ((Flp-Pro-Gly)₇), i.e., an amino acid sequence of: Flp-Pro-Gly-Flp-Pro-Gly-Flp-Pro-Gly-Flp-Pro-Gly-Flp-Pro-Gly-Flp-Pro-Gly-Flp-Pro-Gly (SEQ ID NO:4).

In certain other embodiments of the invention, the collagen mimetic peptide comprises, consists essentially of or consists of an amino acid sequence that is or corresponds to a 21-mer comprising seven repeats of a three amino acid sequence in which Flp has been substituted for proline₂ in SEQ ID NO:1, yielding a sequence of seven repeats of proline-fluoroproline-glycine ((Pro-Flp-Gly)₇), i.e., an amino acid sequence of: Pro-Flp-Gly-Pro-Flp-Gly-Pro-Flp-Gly-Pro-Flp-Gly-Pro-Flp-Gly-Pro-Flp-Gly-Pro-Flp-Gly (SEQ ID NO:5).

In certain other embodiments of the invention, the collagen mimetic peptide comprises, consists essentially of or consists of an amino acid sequence that is or corresponds to a 21-mer comprising seven repeats of a three amino acid sequence in which fluoroproline (Flp) has been substituted for proline₁ in SEQ ID NO:1 and Hyp has been substituted for proline₂ in SEQ ID NO:1, yielding a sequence of seven repeats of fluoroproline-hydroxyproline-glycine ((Flp-Hyp-Gly)₇), i.e., an amino acid sequence of: Flp-Hyp-Gly-Flp-Hyp-Gly-Flp-Hyp-Gly-Flp-Hyp-Gly-Flp-Hyp-Gly-Flp-Hyp-Gly-Flp-Hyp-Gly (SEQ ID NO:6).

In CMPs containing Flp, the Flp moiety may be in the 4-cis or 4-trans configuration, and preferably is in the 4-cis configuration.

In certain other embodiments of the invention, the collagen mimetic peptide may comprise, consist of or have an amino acid sequence that is or corresponds to a 21-mer comprising seven repeats of a three amino acid sequence in which chloroproline (Clp) has been substituted for proline₁ in SEQ ID NO:1, yielding a sequence of seven repeats of chloroproline-proline-glycine ((Clp-Pro-Gly)₇), i.e., an amino acid sequence of: Clp-Pro-Gly-Clp-Pro-Gly-Clp-Pro-Gly-Clp-Pro-Gly-Clp-Pro-Gly-Clp-Pro-Gly-Clp-Pro-Gly (SEQ ID NO:7).

In certain other embodiments of the invention, the collagen mimetic peptide may comprise, consist of or have an amino acid sequence that is or corresponds to a 21-mer comprising seven repeats of a three amino acid sequence in which chloroproline (Clp) has been substituted for proline₂ in SEQ ID NO:1, yielding a sequence of seven repeats of proline-chloroproline-glycine ((Pro-Clp-Gly)₇), i.e., an amino acid sequence of: Pro-Clp-Gly-Pro-Clp-Gly-Pro-Clp-Gly-Pro-Clp-Gly-Pro-Clp-Gly-Pro-Clp-Gly-Pro-Clp-Gly (SEQ ID NO:8).

In certain other embodiments of the invention, the collagen mimetic peptide comprises, consists essentially of or consists of an amino acid sequence that is or corresponds to a 21-mer comprising seven repeats of a three amino acid sequence in which Clp has been substituted for proline₁ in SEQ ID NO:1 and Hyp has been substituted for proline₂ in SEQ ID NO:1, yielding a sequence of seven repeats of chloroproline-hydroxyproline-glycine ((Clp-Hyp-Gly)₇), i.e., an amino acid sequence of: Clp-Hyp-Gly-Clp-Hyp-Gly-Clp-Hyp-Gly-Clp-Hyp-Gly-Clp-Hyp-Gly-Clp-Hyp-Gly-Clp-Hyp-Clp-Gly (SEQ ID NO:9).

In CMPs containing Clp, the Clp moiety may be in the 4-cis or 4-trans configuration, and preferably is in the 4-cis configuration.

In certain other embodiments of the invention, the collagen mimetic peptide may comprise, consist of or have an amino acid sequence that is or corresponds to a 21-mer of any one of SEQ ID NOs:1-9, in which at least one cysteine (Cys) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 10)
Pro-Pro-Gly-Pro-Pro-Gly-Cys-Pro-Gly-Pro-Pro-Gly-Pro-Pro-Gly-Pro-Pro-Gly-Pro-Pro-Gly;
(SEQ ID NO: 11)
Hyp-Pro-Gly-Hyp-Pro-Gly-Cys-Pro-Gly-Hyp-Pro-Gly-Hyp-Pro-Gly-Hyp-Pro-Gly-Hyp-Pro-Gly;
(SEQ ID NO: 12)
Pro-Hyp-Gly-Pro-Hyp-Gly-Pro-Cys-Gly-Pro-Hyp-Gly-Pro-Hyp-Gly-Pro-Hyp-Gly-Pro-Hyp-Gly;
(SEQ ID NO: 13)
Pro-Flp-Gly-Pro-Flp-Gly-Pro-Cys-Gly-Pro-Flp-Gly-Pro-Flp-Gly-Pro-Flp-Gly-Pro-Flp-Gly;
(SEQ ID NO: 14)
Pro-Clp-Gly-Pro-Clp-Gly-Pro-Cys-Gly-Pro-Clp-Gly-Pro-Clp-Gly-Pro-Clp-Gly-Pro-Clp-Gly;
(SEQ ID NO: 15)
Cys-Pro-Gly-Pro-Pro-Gly-Pro-Pro-Gly-Pro-Pro-Gly-Pro-Pro-Gly-Pro-Pro-Gly-Pro-Pro-Gly;
(SEQ ID NO: 16)
Pro-Cys-Gly-Pro-Pro-Gly-Pro-Pro-Gly-Pro-Pro-Gly-Pro-Pro-Gly-Pro-Pro-Gly-Pro-Pro-Gly;
(SEQ ID NO: 17)
Pro-Pro-Gly-Cys-Pro-Gly-Pro-Pro-Gly-Pro-Pro-Gly-Pro-Pro-Gly-Pro-Pro-Gly-Pro-Pro-Gly;
(SEQ ID NO: 18)
Pro-Pro-Gly-Pro-Cys-Gly-Pro-Pro-Gly-Pro-Pro-Gly-Pro-Pro-Gly-Pro-Pro-Gly-Pro-Pro-Gly;
(SEQ ID NO: 19)
Pro-Pro-Gly-Pro-Pro-Gly-Pro-Cys-Gly-Pro-Pro-Gly-Pro-Pro-Gly-Pro-Pro-Gly-Pro-Pro-Gly;
(SEQ ID NO: 20)
Pro-Pro-Gly-Pro-Pro-Gly-Pro-Pro-Gly-Cys-Pro-Gly-Pro-Pro-Gly-Pro-Pro-Gly-Pro-Pro-Gly;
(SEQ ID NO: 21)
Pro-Pro-Gly-Pro-Pro-Gly-Pro-Pro-Gly-Pro-Cys-Gly-Pro-Pro-Gly-Pro-Pro-Gly-Pro-Pro-Gly;
(SEQ ID NO: 22)
Pro-Pro-Gly-Pro-Pro-Gly-Pro-Pro-Gly-Pro-Pro-Gly-Cys-Pro-Gly-Pro-Pro-Gly-Pro-Pro-Gly;
(SEQ ID NO: 23)
Pro-Pro-Gly-Pro-Pro-Gly-Pro-Pro-Gly-Pro-Pro-Gly-Pro-Cys-Gly-Pro-Pro-Gly-Pro-Pro-Gly;
(SEQ ID NO: 24)
Pro-Pro-Gly-Pro-Pro-Gly-Pro-Pro-Gly-Pro-Pro-Gly-Pro-Pro-Gly-Cys-Pro-Gly-Pro-Pro-Gly;
(SEQ ID NO: 25)
Pro-Pro-Gly-Pro-Pro-Gly-Pro-Pro-Gly-Pro-Pro-Gly-Pro-Pro-Gly-Pro-Cys-Gly-Cys-Pro-Gly;
(SEQ ID NO: 26)
Pro-Pro-Gly-Pro-Pro-Gly-Pro-Pro-Gly-Pro-Pro-Gly-Pro-Pro-Gly-Pro-Pro-Gly-Cys-Pro-Gly;
(SEQ ID NO: 27)
Pro-Pro-Gly-Pro-Pro-Gly-Pro-Pro-Gly-Pro-Pro-Gly-Pro-Pro-Gly-Pro-Pro-Gly-Pro-Cys-Gly;
(SEQ ID NO: 28)
Cys-Pro-Gly-Hyp-Pro-Gly-Hyp-Pro-Gly-Hyp-Pro-Gly-Hyp-Pro-Gly-Hyp-Pro-Gly-Hyp-Pro-Gly;
(SEQ ID NO: 29)
Hyp-Cys-Gly-Hyp-Pro-Gly-Hyp-Pro-Gly-Hyp-Pro-Gly-Hyp-Pro-Gly-Hyp-Pro-Gly-Hyp-Pro-Gly;
(SEQ ID NO: 30)
Hyp-Pro-Gly-Cys-Pro-Gly-Hyp-Pro-Gly-Hyp-Pro-Gly-Hyp-Pro-Gly-Hyp-Pro-Gly-Hyp-Pro-Gly;
(SEQ ID NO: 31)
Hyp-Pro-Gly-Hyp-Cys-Gly-Hyp-Pro-Gly-Hyp-Pro-Gly-Hyp-Pro-Gly-Hyp-Pro-Gly-Hyp-Pro-Gly;
(SEQ ID NO: 32)
Hyp-Pro-Gly-Hyp-Pro-Gly-Hyp-Cys-Gly-Hyp-Pro-Gly-Hyp-Pro-Gly-Hyp-Pro-Gly-Hyp-Pro-Gly;
(SEQ ID NO: 33)
Hyp-Pro-Gly-Hyp-Pro-Gly-Hyp-Pro-Gly-Cys-Pro-Gly-Hyp-Pro-Gly-Hyp-Pro-Gly-Hyp-Pro-Gly;
(SEQ ID NO: 34)
Hyp-Pro-Gly-Hyp-Pro-Gly-Hyp-Pro-Gly-Hyp-Cys-Gly-Hyp-Pro-Gly-Hyp-Pro-Gly-Hyp-Pro-Gly;
(SEQ ID NO: 35)
Hyp-Pro-Gly-Hyp-Pro-Gly-Hyp-Pro-Gly-Hyp-Pro-Gly-Cys-Pro-Gly-Hyp-Pro-Gly-Hyp-Pro-Gly;
(SEQ ID NO: 36)
Hyp-Pro-Gly-Hyp-Pro-Gly-Hyp-Pro-Gly-Hyp-Pro-Gly-Hyp-Cys-Gly-Hyp-Pro-Gly-Hyp-Pro-Gly;
(SEQ ID NO: 37)
Hyp-Pro-Gly-Hyp-Pro-Gly-Hyp-Pro-Gly-Hyp-Pro-Gly-Hyp-Pro-Gly-Cys-Pro-Gly-Hyp-Pro-Gly;
(SEQ ID NO: 38)
Hyp-Pro-Gly-Hyp-Pro-Gly-Hyp-Pro-Gly-Hyp-Pro-Gly-Hyp-Pro-Gly-Hyp-Cys-Gly-Hyp-Pro-Gly;
(SEQ ID NO: 39)
Hyp-Pro-Gly-Hyp-Pro-Gly-Hyp-Pro-Gly-Hyp-Pro-Gly-Hyp-Pro-Gly-Hyp-Pro-Gly-Cys-Pro-Gly;
(SEQ ID NO: 40)
Hyp-Pro-Gly-Hyp-Pro-Gly-Hyp-Pro-Gly-Hyp-Pro-Gly-Hyp-Pro-Gly-Hyp-Pro-Gly-Hyp-Cys-Gly;
(SEQ ID NO: 41)
Cys-Hyp-Gly-Pro-Hyp-Gly-Pro-Hyp-Gly-Pro-Hyp-Gly-Pro-Hyp-Gly-Pro-Hyp-Gly-Pro-Hyp-Gly;
(SEQ ID NO: 42)
Pro-Cys-Gly-Pro-Hyp-Gly-Pro-Hyp-Gly-Pro-Hyp-Gly-Pro-Hyp-Gly-Pro-Hyp-Gly-Pro-Hyp-Gly;
(SEQ ID NO: 43)
Pro-Hyp-Gly-Cys-Hyp-Gly-Pro-Hyp-Gly-Pro-Hyp-Gly-Pro-Hyp-Gly-Pro-Hyp-Gly-Pro-Hyp-Gly;
(SEQ ID NO: 44)
Pro-Hyp-Gly-Pro-Cys-Gly-Pro-Hyp-Gly-Pro-Hyp-Gly-Pro-Hyp-Gly-Pro-Hyp-Gly-Pro-Hyp-Gly;
(SEQ ID NO: 45)
Pro-Hyp-Gly-Pro-Hyp-Gly-Cys-Hyp-Gly-Pro-Hyp-Gly-Pro-Hyp-Gly-Pro-Hyp-Gly-Pro-Hyp-Gly;
(SEQ ID NO: 46)
Pro-Hyp-Gly-Pro-Hyp-Gly-Pro-Hyp-Gly-Cys-Hyp-Gly-Pro-Hyp-Gly-Pro-Hyp-Gly-Pro-Hyp-Gly;
(SEQ ID NO: 47)
Pro-Hyp-Gly-Pro-Hyp-Gly-Pro-Hyp-Gly-Pro-Cys-Gly-Pro-Hyp-Gly-Pro-Hyp-Gly-Pro-Hyp-Gly;
(SEQ ID NO: 48)
Pro-Hyp-Gly-Pro-Hyp-Gly-Pro-Hyp-Gly-Pro-Hyp-Gly-Cys-Hyp-Gly-Pro-Hyp-Gly-Pro-Hyp-Gly;
(SEQ ID NO: 49)
Pro-Hyp-Gly-Pro-Hyp-Gly-Pro-Hyp-Gly-Pro-Hyp-Gly-Pro-Cys-Gly-Pro-Hyp-Gly-Pro-Hyp-Gly;
(SEQ ID NO: 50)
Pro-Hyp-Gly-Pro-Hyp-Gly-Pro-Hyp-Gly-Pro-Hyp-Gly-Pro-Hyp-Gly-Cys-Hyp-Gly-Pro-Hyp-Gly;
(SEQ ID NO: 51)
Pro-Hyp-Gly-Pro-Hyp-Gly-Pro-Hyp-Gly-Pro-Hyp-Gly-Pro-Hyp-Gly-Pro-Cys-Gly-Pro-Hyp-Gly;
(SEQ ID NO: 52)
Pro-Hyp-Gly-Pro-Hyp-Gly-Pro-Hyp-Gly-Pro-Hyp-Gly-Pro-Hyp-Gly-Pro-Hyp-Gly-Cys-Hyp-Gly;
(SEQ ID NO: 53)
Pro-Hyp-Gly-Pro-Hyp-Gly-Pro-Hyp-Gly-Pro-Hyp-Gly-Pro-Hyp-Gly-Pro-Hyp-Gly-Pro-Cys-Gly;
(SEQ ID NO: 54)
Cys-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 55)
Pro-Cys-Gly-Pro-Flp-Gly-Pro-Flp-Gly-Pro-Flp-Gly-Pro-Flp-Gly-Pro-Flp-Gly-Pro-Flp-Gly;
(SEQ ID NO: 56)
Pro-Flp-Gly-Cys-Flp-Gly-Pro-Flp-Gly-Pro-Flp-Gly-Pro-Flp-Gly-Pro-Flp-Gly-Pro-Flp-Gly;
(SEQ ID NO: 57)
Pro-Flp-Gly-Pro-Cys-Gly-Pro-Flp-Gly-Pro-Flp-Gly-Pro-Flp-Gly-Pro-Flp-Gly-Pro-Flp-Gly;
(SEQ ID NO: 58)
Pro-Flp-Gly-Pro-Flp-Gly-Cys-Flp-Gly-Pro-Flp-Gly-Pro-Flp-Gly-Pro-Flp-Gly-Pro-Flp-Gly;
(SEQ ID NO: 59)
Pro-Flp-Gly-Pro-Flp-Gly-Pro-Flp-Gly-Cys-Flp-Gly-Pro-Flp-Gly-Pro-Flp-Gly-Pro-Flp-Gly;
(SEQ ID NO: 60)
Pro-Flp-Gly-Pro-Flp-Gly-Pro-Flp-Gly-Pro-Cys-Gly-Pro-Flp-Gly-Pro-Flp-Gly-Pro-Flp-Gly;
(SEQ ID NO: 61)
Pro-Flp-Gly-Pro-Flp-Gly-Pro-Flp-Gly-Pro-Flp-Gly-Cys-Flp-Gly-Pro-Flp-Gly-Pro-Flp-Gly;
(SEQ ID NO: 62)
Pro-Flp-Gly-Pro-Flp-Gly-Pro-Flp-Gly-Pro-Flp-Gly-Pro-Cys-Gly-Pro-Flp-Gly-Pro-Flp-Gly;
(SEQ ID NO: 63)
Pro-Flp-Gly-Pro-Flp-Gly-Pro-Flp-Gly-Pro-Flp-Gly-Pro-Flp-Gly-Cys-Flp-Gly-Pro-Flp-Gly;
(SEQ ID NO: 64)
Pro-Flp-Gly-Pro-Flp-Gly-Pro-Flp-Gly-Pro-Flp-Gly-Pro-Flp-Gly-Pro-Cys-Gly-Pro-Flp-Gly;
(SEQ ID NO: 65)
Pro-Flp-Gly-Pro-Flp-Gly-Pro-Flp-Gly-Pro-Flp-Gly-Pro-Flp-Gly-Pro-Flp-Gly-Cys-Flp-Gly;
(SEQ ID NO: 66)
Pro-Flp-Gly-Pro-Flp-Gly-Pro-Flp-Gly-Pro-Flp-Gly-Pro-Flp-Gly-Pro-Flp-Gly-Pro-Cys-Gly;
(SEQ ID NO: 67)
Cys-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 68)
Pro-Cys-Gly-Pro-Flp-Gly-Pro-Flp-Gly-Pro-Flp-Gly-Pro-Flp-Gly-Pro-Flp-Gly-Pro-Flp-Gly;
(SEQ ID NO: 69)
Pro-Flp-Gly-Cys-Flp-Gly-Pro-Flp-Gly-Pro-Flp-Gly-Pro-Flp-Gly-Pro-Flp-Gly-Pro-Flp-Gly;
(SEQ ID NO: 70)
Pro-Flp-Gly-Pro-Cys-Gly-Pro-Flp-Gly-Pro-Flp-Gly-Pro-Flp-Gly-Pro-Flp-Gly-Pro-Flp-Gly;
(SEQ ID NO: 71)
Pro-Flp-Gly-Pro-Flp-Gly-Cys-Flp-Gly-Pro-Flp-Gly-Pro-Flp-Gly-Pro-Flp-Gly-Pro-Flp-Gly;
(SEQ ID NO: 72)
Pro-Flp-Gly-Pro-Flp-Gly-Pro-Cys-Gly-Pro-Flp-Gly-Pro-Flp-Gly-Pro-Flp-Gly-Pro-Flp-Gly;
(SEQ ID NO: 73)
Pro-Flp-Gly-Pro-Flp-Gly-Pro-Flp-Gly-Cys-Flp-Gly-Pro-Flp-Gly-Pro-Flp-Gly-Pro-Flp-Gly;
(SEQ ID NO: 74)
Pro-Flp-Gly-Pro-Flp-Gly-Pro-Flp-Gly-Pro-Cys-Gly-Pro-Flp-Gly-Pro-Flp-Gly-Pro-Flp-Gly;
(SEQ ID NO: 75)
Pro-Flp-Gly-Pro-Flp-Gly-Pro-Flp-Gly-Pro-Flp-Gly-Cys-Flp-Gly-Pro-Flp-Gly-Pro-Flp-Gly;
(SEQ ID NO: 76)
Pro-Flp-Gly-Pro-Flp-Gly-Pro-Flp-Gly-Pro-Flp-Gly-Pro-Cys-Gly-Pro-Flp-Gly-Pro-Flp-Gly;
(SEQ ID NO: 77)
Pro-Flp-Gly-Pro-Flp-Gly-Pro-Flp-Gly-Pro-Flp-Gly-Pro-Flp-Gly-Cys-Flp-Gly-Pro-Flp-Gly;
(SEQ ID NO: 78)
Pro-Flp-Gly-Pro-Flp-Gly-Pro-Flp-Gly-Pro-Flp-Gly-Pro-Flp-Gly-Pro-Cys-Gly-Pro-Flp-Gly;
(SEQ ID NO: 79)
Pro-Flp-Gly-Pro-Flp-Gly-Pro-Flp-Gly-Pro-Flp-Gly-Pro-Flp-Gly-Pro-Flp-Gly-Cys-Flp-Gly;
(SEQ ID NO: 80)
Pro-Flp-Gly-Pro-Flp-Gly-Pro-Flp-Gly-Pro-Flp-Gly-Pro-Flp-Gly-Pro-Flp-Gly-Pro-Cys-Gly;
(SEQ ID NO: 81)
Cys-Hyp-Gly-Flp-Hyp-Gly-Flp-Hyp-Gly-Flp-Hyp-Gly-Flp-Hyp-Gly-Flp-Hyp-Gly-Flp-Hyp-Gly;
(SEQ ID NO: 82)
Flp-Cys-Gly-Flp-Hyp-Gly-Flp-Hyp-Gly-Flp-Hyp-Gly-Flp-Hyp-Gly-Flp-Hyp-Gly-Flp-Hyp-Gly;
(SEQ ID NO: 83)
Flp-Hyp-Gly-Cys-Hyp-Gly-Flp-Hyp-Gly-Flp-Hyp-Gly-Flp-Hyp-Gly-Flp-Hyp-Gly-Flp-Hyp-Gly;
(SEQ ID NO: 84)
Flp-Hyp-Gly-Flp-Cys-Gly-Flp-Hyp-Gly-Flp-Hyp-Gly-Flp-Hyp-Gly-Flp-Hyp-Gly-Flp-Hyp-Gly;
(SEQ ID NO: 85)
Flp-Hyp-Gly-Flp-Hyp-Gly-Cys-Hyp-Gly-Flp-Hyp-Gly-Flp-Hyp-Gly-Flp-Hyp-Gly-Flp-Hyp-Gly;
(SEQ ID NO: 86)
Flp-Hyp-Gly-Flp-Hyp-Gly-Flp-Cys-Gly-Flp-Hyp-Gly-Flp-Hyp-Gly-Flp-Hyp-Gly-Flp-Hyp-Gly;
(SEQ ID NO: 87)
Flp-Hyp-Gly-Flp-Hyp-Gly-Flp-Hyp-Gly-Cys-Hyp-Gly-Flp-Hyp-Gly-Flp-Hyp-Gly-Flp-Hyp-Gly;
(SEQ ID NO: 88)
Flp-Hyp-Gly-Flp-Hyp-Gly-Flp-Hyp-Gly-Flp-Cys-Gly-Flp-Hyp-Gly-Flp-Hyp-Gly-Flp-Hyp-Gly;
(SEQ ID NO: 89)
Flp-Hyp-Gly-Flp-Hyp-Gly-Flp-Hyp-Gly-Flp-Hyp-Gly-Cys-Hyp-Gly-Flp-Hyp-Gly-Flp-Hyp-Gly;
(SEQ ID NO: 90)
Flp-Hyp-Gly-Flp-Hyp-Gly-Flp-Hyp-Gly-Flp-Hyp-Gly-Flp-Cys-Gly-Flp-Hyp-Gly-Flp-Hyp-Gly;
(SEQ ID NO: 91)
Flp-Hyp-Gly-Flp-Hyp-Gly-Flp-Hyp-Gly-Flp-Hyp-Gly-Flp-Hyp-Gly-Cys-Hyp-Gly-Flp-Hyp-Gly;
(SEQ ID NO: 92)
Flp-Hyp-Gly-Flp-Hyp-Gly-Flp-Hyp-Gly-Flp-Hyp-Gly-Flp-Hyp-Gly-Flp-Cys-Gly-Flp-Hyp-Gly;
(SEQ ID NO: 93)
Flp-Hyp-Gly-Flp-Hyp-Gly-Flp-Hyp-Gly-Flp-Hyp-Gly-Flp-Hyp-Gly-Flp-Hyp-Gly-Cys-Hyp-Gly;
(SEQ ID NO: 94)
Flp-Hyp-Gly-Flp-Hyp-Gly-Flp-Hyp-Gly-Flp-Hyp-Gly-Flp-Hyp-Gly-Flp-Hyp-Gly-Flp-Cys-Gly;
(SEQ ID NO: 95)
Cys-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 96)
Pro-Cys-Gly-Pro-Clp-Gly-Pro-Clp-Gly-Pro-Clp-Gly-Pro-Clp-Gly-Pro-Clp-Gly-Pro-Clp-Gly;
(SEQ ID NO: 97)
Pro-Clp-Gly-Cys-Clp-Gly-Pro-Clp-Gly-Pro-Clp-Gly-Pro-Clp-Gly-Pro-Clp-Gly-Pro-Clp-Gly;
(SEQ ID NO: 98)
Pro-Clp-Gly-Pro-Cys-Gly-Pro-Clp-Gly-Pro-Clp-Gly-Pro-Clp-Gly-Pro-Clp-Gly-Pro-Clp-Gly;
(SEQ ID NO: 99)
Pro-Clp-Gly-Pro-Clp-Gly-Cys-Clp-Gly-Pro-Clp-Gly-Pro-Clp-Gly-Pro-Clp-Gly-Pro-Clp-Gly;
(SEQ ID NO: 100)
Pro-Clp-Gly-Pro-Clp-Gly-Pro-Clp-Gly-Cys-Clp-Gly-Pro-Clp-Gly-Pro-Clp-Gly-Pro-Clp-Gly;
(SEQ ID NO: 101)
Pro-Clp-Gly-Pro-Clp-Gly-Pro-Clp-Gly-Pro-Cys-Gly-Pro-Clp-Gly-Pro-Clp-Gly-Pro-Clp-Gly;
(SEQ ID NO: 102)
Pro-Clp-Gly-Pro-Clp-Gly-Pro-Clp-Gly-Pro-Clp-Gly-Cys-Clp-Gly-Pro-Clp-Gly-Pro-Clp-Gly;
(SEQ ID NO: 103)
Pro-Clp-Gly-Pro-Clp-Gly-Pro-Clp-Gly-Pro-Clp-Gly-Pro-Cys-Gly-Pro-Clp-Gly-Pro-Clp-Gly;
(SEQ ID NO: 104)
Pro-Clp-Gly-Pro-Clp-Gly-Pro-Clp-Gly-Pro-Clp-Gly-Pro-Clp-Gly-Cys-Clp-Gly-Pro-Clp-Gly;
(SEQ ID NO: 105)
Pro-Clp-Gly-Pro-Clp-Gly-Pro-Clp-Gly-Pro-Clp-Gly-Pro-Clp-Gly-Pro-Cys-Gly-Pro-Clp-Gly;
(SEQ ID NO: 106)
Pro-Clp-Gly-Pro-Clp-Gly-Pro-Clp-Gly-Pro-Clp-Gly-Pro-Clp-Gly-Pro-Clp-Gly-Cys-Clp-Gly;
(SEQ ID NO: 107)
Pro-Clp-Gly-Pro-Clp-Gly-Pro-Clp-Gly-Pro-Clp-Gly-Pro-Clp-Gly-Pro-Clp-Gly-Pro-Cys-Gly;
(SEQ ID NO: 108)
Cys-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 109)
Pro-Cys-Gly-Pro-Clp-Gly-Pro-Clp-Gly-Pro-Clp-Gly-Pro-Clp-Gly-Pro-Clp-Gly-Pro-Clp-Gly;
(SEQ ID NO: 110)
Pro-Clp-Gly-Cys-Clp-Gly-Pro-Clp-Gly-Pro-Clp-Gly-Pro-Clp-Gly-Pro-Clp-Gly-Pro-Clp-Gly;
(SEQ ID NO: 111)
Pro-Clp-Gly-Pro-Cys-Gly-Pro-Clp-Gly-Pro-Clp-Gly-Pro-Clp-Gly-Pro-Clp-Gly-Pro-Clp-Gly;
(SEQ ID NO: 112)
Pro-Clp-Gly-Pro-Clp-Gly-Cys-Clp-Gly-Pro-Clp-Gly-Pro-Clp-Gly-Pro-Clp-Gly-Pro-Clp-Gly;
(SEQ ID NO: 113)
Pro-Clp-Gly-Pro-Clp-Gly-Pro-Cys-Gly-Pro-Clp-Gly-Pro-Clp-Gly-Pro-Clp-Gly-Pro-Clp-Gly;
(SEQ ID NO: 114)
Pro-Clp-Gly-Pro-Clp-Gly-Pro-Clp-Gly-Cys-Clp-Gly-Pro-Clp-Gly-Pro-Clp-Gly-Pro-Clp-Gly;
(SEQ ID NO: 115)
Pro-Clp-Gly-Pro-Clp-Gly-Pro-Clp-Gly-Pro-Cys-Gly-Pro-Clp-Gly-Pro-Clp-Gly-Pro-Clp-Gly;
(SEQ ID NO: 116)
Pro-Clp-Gly-Pro-Clp-Gly-Pro-Clp-Gly-Pro-Clp-Gly-Cys-Clp-Gly-Pro-Clp-Gly-Pro-Clp-Gly;
(SEQ ID NO: 117)
Pro-Clp-Gly-Pro-Clp-Gly-Pro-Clp-Gly-Pro-Clp-Gly-Pro-Cys-Gly-Pro-Clp-Gly-Pro-Clp-Gly;
(SEQ ID NO: 118)
Pro-Clp-Gly-Pro-Clp-Gly-Pro-Clp-Gly-Pro-Clp-Gly-Pro-Clp-Gly-Cys-Clp-Gly-Pro-Clp-Gly;
(SEQ ID NO: 119)
Pro-Clp-Gly-Pro-Clp-Gly-Pro-Clp-Gly-Pro-Clp-Gly-Pro-Clp-Gly-Pro-Cys-Gly-Pro-Clp-Gly;
(SEQ ID NO: 120)
Pro-Clp-Gly-Pro-Clp-Gly-Pro-Clp-Gly-Pro-Clp-Gly-Pro-Clp-Gly-Pro-Clp-Gly-Cys-Clp-Gly;
(SEQ ID NO: 121)
Pro-Clp-Gly-Pro-Clp-Gly-Pro-Clp-Gly-Pro-Clp-Gly-Pro-Clp-Gly-Pro-Clp-Gly-Pro-Cys-Gly;
(SEQ ID NO: 122)
Cys-Hyp-Gly-Clp-Hyp-Gly-Clp-Hyp-Gly-Clp-Hyp-Gly-Clp-Hyp-Gly-Clp-Hyp-Gly-Clp-Hyp-Gly;
(SEQ ID NO: 123)
Clp-Cys-Gly-Clp-Hyp-Gly-Clp-Hyp-Gly-Clp-Hyp-Gly-Clp-Hyp-Gly-Clp-Hyp-Gly-Clp-Hyp-Gly;
(SEQ ID NO: 124)
Clp-Hyp-Gly-Cys-Hyp-Gly-Clp-Hyp-Gly-Clp-Hyp-Gly-Clp-Hyp-Gly-Clp-Hyp-Gly-Clp-Hyp-Gly;
(SEQ ID NO: 125)
Clp-Hyp-Gly-Clp-Cys-Gly-Clp-Hyp-Gly-Clp-Hyp-Gly-Clp-Hyp-Gly-Clp-Hyp-Gly-Clp-Hyp-Gly;
(SEQ ID NO: 126)
Clp-Hyp-Gly-Clp-Hyp-Gly-Cys-Hyp-Gly-Clp-Hyp-Gly-Clp-Hyp-Gly-Clp-Hyp-Gly-Clp-Hyp-Gly;
(SEQ ID NO: 127)
Clp-Hyp-Gly-Clp-Hyp-Gly-Clp-Cys-Gly-Clp-Hyp-Gly-Clp-Hyp-Gly-Clp-Hyp-Gly-Clp-Hyp-Gly;
(SEQ ID NO: 128)
Clp-Hyp-Gly-Clp-Hyp-Gly-Clp-Hyp-Gly-Cys-Hyp-Gly-Clp-Hyp-Gly-Clp-Hyp-Gly-Clp-Hyp-Gly;
(SEQ ID NO: 129)
Clp-Hyp-Gly-Clp-Hyp-Gly-Clp-Hyp-Gly-Clp-Cys-Gly-Clp-Hyp-Gly-Clp-Hyp-Gly-Clp-Hyp-Gly;
(SEQ ID NO: 130)
Clp-Hyp-Gly-Clp-Hyp-Gly-Clp-Hyp-Gly-Clp-Hyp-Gly-Cys-Hyp-Gly-Clp-Hyp-Gly-Clp-Hyp-Gly;
(SEQ ID NO: 131)
Clp-Hyp-Gly-Clp-Hyp-Gly-Clp-Hyp-Gly-Clp-Hyp-Gly-Clp-Cys-Gly-Clp-Hyp-Gly-Clp-Hyp-Gly;
(SEQ ID NO: 132)
Clp-Hyp-Gly-Clp-Hyp-Gly-Clp-Hyp-Gly-Clp-Hyp-Gly-Clp-Hyp-Gly-Cys-Hyp-Gly-Clp-Hyp-Gly;
(SEQ ID NO: 133)
Clp-Hyp-Gly-Clp-Hyp-Gly-Clp-Hyp-Gly-Clp-Hyp-Gly-Clp-Hyp-Gly-Clp-Cys-Gly-Clp-Hyp-Gly;
(SEQ ID NO: 134)
Clp-Hyp-Gly-Clp-Hyp-Gly-Clp-Hyp-Gly-Clp-Hyp-Gly-Clp-Hyp-Gly-Clp-Hyp-Gly-Cys-Hyp-Gly;
and
(SEQ ID NO: 135)
Clp-Hyp-Gly-Clp-Hyp-Gly-Clp-Hyp-Gly-Clp-Hyp-Gly-Clp-Hyp-Gly-Clp-Hyp-Gly-Clp-Cys-Gly.

In certain other embodiments of the invention, the collagen mimetic peptide may comprise, consist of or have an amino acid sequence that is or corresponds to a 21-mer of any one of SEQ ID NOs:1-9, in which at least one methionine (Met) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 136)
Pro-Pro-Gly-Pro-Pro-Gly-Met-Pro-Gly-Pro-Pro-Gly-Pro-Pro-Gly-Pro-Pro-Gly-Pro-Pro-Gly;
(SEQ ID NO: 137)
Hyp-Pro-Gly-Hyp-Pro-Gly-Met-Pro-Gly-Hyp-Pro-Gly-Hyp-Pro-Gly-Hyp-Pro-Gly-Hyp-Pro-
Gly;
(SEQ ID NO: 138)
Pro-Hyp-Gly-Pro-Hyp-Gly-Pro-Met-Gly-Pro-Hyp-Gly-Pro-Hyp-Gly-Pro-Hyp-Gly-Pro-Hyp-
Gly;
(SEQ ID NO: 139)
Pro-Flp-Gly-Pro-Flp-Gly-Pro-Met-Gly-Pro-Flp-Gly-Pro-Flp-Gly-Pro-Flp-Gly-Pro-Flp-Gly;
(SEQ ID NO: 140)
Pro-Clp-Gly-Pro-Clp-Gly-Pro-Met-Gly-Pro-Clp-Gly-Pro-Clp-Gly-Pro-Clp-Gly-Pro-Clp-Gly;
(SEQ ID NO: 141)
Met-Pro-Gly-Pro-Pro-Gly-Pro-Pro-Gly-Pro-Pro-Gly-Pro-Pro-Gly-Pro-Pro-Gly-Pro-Pro-Gly;
(SEQ ID NO: 142)
Pro-Met-Gly-Pro-Pro-Gly-Pro-Pro-Gly-Pro-Pro-Gly-Pro-Pro-Gly-Pro-Pro-Gly-Pro-Pro-Gly;
(SEQ ID NO: 143)
Pro-Pro-Gly-Met-Pro-Gly-Pro-Pro-Gly-Pro-Pro-Gly-Pro-Pro-Gly-Pro-Pro-Gly-Pro-Pro-Gly;
(SEQ ID NO: 144)
Pro-Pro-Gly-Pro-Met-Gly-Pro-Pro-Gly-Pro-Pro-Gly-Pro-Pro-Gly-Pro-Pro-Gly-Pro-Pro-Gly;
(SEQ ID NO: 145)
Pro-Pro-Gly-Pro-Pro-Gly-Pro-Met-Gly-Pro-Pro-Gly-Pro-Pro-Gly-Pro-Pro-Gly-Pro-Pro-Gly;
(SEQ ID NO: 146)
Pro-Pro-Gly-Pro-Pro-Gly-Pro-Pro-Gly-Met-Pro-Gly-Pro-Pro-Gly-Pro-Pro-Gly-Pro-Pro-Gly;
(SEQ ID NO: 147)
Pro-Pro-Gly-Pro-Pro-Gly-Pro-Pro-Gly-Pro-Met-Gly-Pro-Pro-Gly-Pro-Pro-Gly-Pro-Pro-Gly;
(SEQ ID NO: 148)
Pro-Pro-Gly-Pro-Pro-Gly-Pro-Pro-Gly-Pro-Pro-Gly-Met-Pro-Gly-Pro-Pro-Gly-Pro-Pro-Gly;
(SEQ ID NO: 149)
Pro-Pro-Gly-Pro-Pro-Gly-Pro-Pro-Gly-Pro-Pro-Gly-Pro-Met-Gly-Pro-Pro-Gly-Pro-Pro-Gly;
(SEQ ID NO: 150)
Pro-Pro-Gly-Pro-Pro-Gly-Pro-Pro-Gly-Pro-Pro-Gly-Pro-Pro-Gly-Met-Pro-Gly-Pro-Pro-Gly;
(SEQ ID NO: 151)
Pro-Pro-Gly-Pro-Pro-Gly-Pro-Pro-Gly-Pro-Pro-Gly-Pro-Pro-Gly-Pro-Met-Gly-Pro-Pro-Gly;
(SEQ ID NO: 152)
Pro-Pro-Gly-Pro-Pro-Gly-Pro-Pro-Gly-Pro-Pro-Gly-Pro-Pro-Gly-Pro-Pro-Gly-Met-Pro-Gly;
(SEQ ID NO: 153)
Pro-Pro-Gly-Pro-Pro-Gly-Pro-Pro-Gly-Pro-Pro-Gly-Pro-Pro-Gly-Pro-Pro-Gly-Pro-Met-Gly;
(SEQ ID NO: 154)
Met-Pro-Gly-Hyp-Pro-Gly-Hyp-Pro-Gly-Hyp-Pro-Gly-Hyp-Pro-Gly-Hyp-Pro-Gly-Hyp-Pro-
Gly;
(SEQ ID NO: 155)
Hyp-Met-Gly-Hyp-Pro-Gly-Hyp-Pro-Gly-Hyp-Pro-Gly-Hyp-Pro-Gly-Hyp-Pro-Gly-Hyp-
Pro-Gly;
(SEQ ID NO: 156)
Hyp-Pro-Gly-Met-Pro-Gly-Hyp-Pro-Gly-Hyp-Pro-Gly-Hyp-Pro-Gly-Hyp-Pro-Gly-Hyp-Pro-
Gly;
(SEQ ID NO: 157)
Hyp-Pro-Gly-Hyp-Met-Gly-Hyp-Pro-Gly-Hyp-Pro-Gly-Hyp-Pro-Gly-Hyp-Pro-Gly-Hyp-
Pro-Gly;
(SEQ ID NO: 158)
Hyp-Pro-Gly-Hyp-Pro-Gly-Hyp-Met-Gly-Hyp-Pro-Gly-Hyp-Pro-Gly-Hyp-Pro-Gly-Hyp-
Pro-Gly;
(SEQ ID NO: 159)
Hyp-Pro-Gly-Hyp-Pro-Gly-Hyp-Pro-Gly-Met-Pro-Gly-Hyp-Pro-Gly-Hyp-Pro-Gly-Hyp-Pro-
Gly;
(SEQ ID NO: 160)
Hyp-Pro-Gly-Hyp-Pro-Gly-Hyp-Pro-Gly-Hyp-Met-Gly-Hyp-Pro-Gly-Hyp-Pro-Gly-Hyp-
Pro-Gly;
(SEQ ID NO: 161)
Hyp-Pro-Gly-Hyp-Pro-Gly-Hyp-Pro-Gly-Hyp-Pro-Gly-Met-Pro-Gly-Hyp-Pro-Gly-Hyp-Pro-
Gly;
(SEQ ID NO: 162)
Hyp-Pro-Gly-Hyp-Pro-Gly-Hyp-Pro-Gly-Hyp-Pro-Gly-Hyp-Met-Gly-Hyp-Pro-Gly-Hyp-
Pro-Gly;
(SEQ ID NO: 163)
Hyp-Pro-Gly-Hyp-Pro-Gly-Hyp-Pro-Gly-Hyp-Pro-Gly-Hyp-Pro-Gly-Met-Pro-Gly-Hyp-Pro-
Gly;
(SEQ ID NO: 164)
Hyp-Pro-Gly-Hyp-Pro-Gly-Hyp-Pro-Gly-Hyp-Pro-Gly-Hyp-Pro-Gly-Hyp-Met-Gly-Hyp-
Pro-Gly;
(SEQ ID NO: 165)
Hyp-Pro-Gly-Hyp-Pro-Gly-Hyp-Pro-Gly-Hyp-Pro-Gly-Hyp-Pro-Gly-Hyp-Pro-Gly-Met-Pro-
Gly;
(SEQ ID NO: 166)
Hyp-Pro-Gly-Hyp-Pro-Gly-Hyp-Pro-Gly-Hyp-Pro-Gly-Hyp-Pro-Gly-Hyp-Pro-Gly-Hyp-
Met-Gly;
(SEQ ID NO: 167)
Met-Hyp-Gly-Pro-Hyp-Gly-Pro-Hyp-Gly-Pro-Hyp-Gly-Pro-Hyp-Gly-Pro-Hyp-Gly-Pro-
Hyp-Gly;
(SEQ ID NO: 168)
Pro-Met-Gly-Pro-Hyp-Gly-Pro-Hyp-Gly-Pro-Hyp-Gly-Pro-Hyp-Gly-Pro-Hyp-Gly-Pro-Hyp-
Gly;
(SEQ ID NO: 169)
Pro-Hyp-Gly-Met-Hyp-Gly-Pro-Hyp-Gly-Pro-Hyp-Gly-Pro-Hyp-Gly-Pro-Hyp-Gly-Pro-
Hyp-Gly;
(SEQ ID NO: 170)
Pro-Hyp-Gly-Pro-Met-Gly-Pro-Hyp-Gly-Pro-Hyp-Gly-Pro-Hyp-Gly-Pro-Hyp-Gly-Pro-Hyp-
Gly;
(SEQ ID NO: 171)
Pro-Hyp-Gly-Pro-Hyp-Gly-Met-Hyp-Gly-Pro-Hyp-Gly-Pro-Hyp-Gly-Pro-Hyp-Gly-Pro-
Hyp-Gly;
(SEQ ID NO: 172)
Pro-Hyp-Gly-Pro-Hyp-Gly-Pro-Hyp-Gly-Met-Hyp-Gly-Pro-Hyp-Gly-Pro-Hyp-Gly-Pro-
Hyp-Gly;
(SEQ ID NO: 173)
Pro-Hyp-Gly-Pro-Hyp-Gly-Pro-Hyp-Gly-Pro-Met-Gly-Pro-Hyp-Gly-Pro-Hyp-Gly-Pro-Hyp-
Gly;
(SEQ ID NO: 174)
Pro-Hyp-Gly-Pro-Hyp-Gly-Pro-Hyp-Gly-Pro-Hyp-Gly-Met-Hyp-Gly-Pro-Hyp-Gly-Pro-
Hyp-Gly;
(SEQ ID NO: 175)
Pro-Hyp-Gly-Pro-Hyp-Gly-Pro-Hyp-Gly-Pro-Hyp-Gly-Pro-Met-Gly-Pro-Hyp-Gly-Pro-Hyp-
Gly;
(SEQ ID NO: 176)
Pro-Hyp-Gly-Pro-Hyp-Gly-Pro-Hyp-Gly-Pro-Hyp-Gly-Pro-Hyp-Gly-Met-Hyp-Gly-Pro-
Hyp-Gly;
(SEQ ID NO: 177)
Pro-Hyp-Gly-Pro-Hyp-Gly-Pro-Hyp-Gly-Pro-Hyp-Gly-Pro-Hyp-Gly-Pro-Met-Gly-Pro-Hyp-
Gly;
(SEQ ID NO: 178)
Pro-Hyp-Gly-Pro-Hyp-Gly-Pro-Hyp-Gly-Pro-Hyp-Gly-Pro-Hyp-Gly-Pro-Hyp-Gly-Met-
Hyp-Gly;
(SEQ ID NO: 179)
Pro-Hyp-Gly-Pro-Hyp-Gly-Pro-Hyp-Gly-Pro-Hyp-Gly-Pro-Hyp-Gly-Pro-Hyp-Gly-Pro-Met-
Gly;
(SEQ ID NO: 180)
Met-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 181)
Pro-Met-Gly-Pro-Flp-Gly-Pro-Flp-Gly-Pro-Flp-Gly-Pro-Flp-Gly-Pro-Flp-Gly-Pro-Flp-Gly;
(SEQ ID NO: 182)
Pro-Flp-Gly-Met-Flp-Gly-Pro-Flp-Gly-Pro-Flp-Gly-Pro-Flp-Gly-Pro-Flp-Gly-Pro-Flp-Gly;
(SEQ ID NO: 183)
Pro-Flp-Gly-Pro-Met-Gly-Pro-Flp-Gly-Pro-Flp-Gly-Pro-Flp-Gly-Pro-Flp-Gly-Pro-Flp-Gly;
(SEQ ID NO: 184)
Pro-Flp-Gly-Pro-Flp-Gly-Met-Flp-Gly-Pro-Flp-Gly-Pro-Flp-Gly-Pro-Flp-Gly-Pro-Flp-Gly;
(SEQ ID NO: 185)
Pro-Flp-Gly-Pro-Flp-Gly-Pro-Flp-Gly-Met-Flp-Gly-Pro-Flp-Gly-Pro-Flp-Gly-Pro-Flp-Gly;
(SEQ ID NO: 186)
Pro-Flp-Gly-Pro-Flp-Gly-Pro-Flp-Gly-Pro-Met-Gly-Pro-Flp-Gly-Pro-Flp-Gly-Pro-Flp-Gly;
(SEQ ID NO: 187)
Pro-Flp-Gly-Pro-Flp-Gly-Pro-Flp-Gly-Pro-Flp-Gly-Met-Flp-Gly-Pro-Flp-Gly-Pro-Flp-Gly;
(SEQ ID NO: 188)
Pro-Flp-Gly-Pro-Flp-Gly-Pro-Flp-Gly-Pro-Flp-Gly-Pro-Met-Gly-Pro-Flp-Gly-Pro-Flp-Gly;
(SEQ ID NO: 189)
Pro-Flp-Gly-Pro-Flp-Gly-Pro-Flp-Gly-Pro-Flp-Gly-Pro-Flp-Gly-Met-Flp-Gly-Pro-Flp-Gly;
(SEQ ID NO: 190)
Pro-Flp-Gly-Pro-Flp-Gly-Pro-Flp-Gly-Pro-Flp-Gly-Pro-Flp-Gly-Pro-Met-Gly-Pro-Flp-Gly;
(SEQ ID NO: 191)
Pro-Flp-Gly-Pro-Flp-Gly-Pro-Flp-Gly-Pro-Flp-Gly-Pro-Flp-Gly-Pro-Flp-Gly-Met-Flp-Gly;
(SEQ ID NO: 192)
Pro-Flp-Gly-Pro-Flp-Gly-Pro-Flp-Gly-Pro-Flp-Gly-Pro-Flp-Gly-Pro-Flp-Gly-Pro-Met-Gly;
(SEQ ID NO: 193)
Met-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 194)
Pro-Met-Gly-Pro-Flp-Gly-Pro-Flp-Gly-Pro-Flp-Gly-Pro-Flp-Gly-Pro-Flp-Gly-Pro-Flp-Gly;
(SEQ ID NO: 195)
Pro-Flp-Gly-Met-Flp-Gly-Pro-Flp-Gly-Pro-Flp-Gly-Pro-Flp-Gly-Pro-Flp-Gly-Pro-Flp-Gly;
(SEQ ID NO: 196)
Pro-Flp-Gly-Pro-Met-Gly-Pro-Flp-Gly-Pro-Flp-Gly-Pro-Flp-Gly-Pro-Flp-Gly-Pro-Flp-Gly;
(SEQ ID NO: 197)
Pro-Flp-Gly-Pro-Flp-Gly-Met-Flp-Gly-Pro-Flp-Gly-Pro-Flp-Gly-Pro-Flp-Gly-Pro-Flp-Gly;
(SEQ ID NO: 198)
Pro-Flp-Gly-Pro-Flp-Gly-Pro-Met-Gly-Pro-Flp-Gly-Pro-Flp-Gly-Pro-Flp-Gly-Pro-Flp-Gly;
(SEQ ID NO: 199)
Pro-Flp-Gly-Pro-Flp-Gly-Pro-Flp-Gly-Met-Flp-Gly-Pro-Flp-Gly-Pro-Flp-Gly-Pro-Flp-Gly;
(SEQ ID NO: 200)
Pro-Flp-Gly-Pro-Flp-Gly-Pro-Flp-Gly-Pro-Met-Gly-Pro-Flp-Gly-Pro-Flp-Gly-Pro-Flp-Gly;
(SEQ ID NO: 201)
Pro-Flp-Gly-Pro-Flp-Gly-Pro-Flp-Gly-Pro-Flp-Gly-Met-Flp-Gly-Pro-Flp-Gly-Pro-Flp-Gly;
(SEQ ID NO: 202)
Pro-Flp-Gly-Pro-Flp-Gly-Pro-Flp-Gly-Pro-Flp-Gly-Pro-Met-Gly-Pro-Flp-Gly-Pro-Flp-Gly;
(SEQ ID NO: 203)
Pro-Flp-Gly-Pro-Flp-Gly-Pro-Flp-Gly-Pro-Flp-Gly-Pro-Flp-Gly-Met-Flp-Gly-Pro-Flp-Gly;
(SEQ ID NO: 204)
Pro-Flp-Gly-Pro-Flp-Gly-Pro-Flp-Gly-Pro-Flp-Gly-Pro-Flp-Gly-Pro-Met-Gly-Pro-Flp-Gly;
(SEQ ID NO: 205)
Pro-Flp-Gly-Pro-Flp-Gly-Pro-Flp-Gly-Pro-Flp-Gly-Pro-Flp-Gly-Pro-Flp-Gly-Met-Flp-Gly;
(SEQ ID NO: 206)
Pro-Flp-Gly-Pro-Flp-Gly-Pro-Flp-Gly-Pro-Flp-Gly-Pro-Flp-Gly-Pro-Flp-Gly-Pro-Met-Gly;
(SEQ ID NO: 207)
Met-Hyp-Gly-Flp-Hyp-Gly-Flp-Hyp-Gly-Flp-Hyp-Gly-Flp-Hyp-Gly-Flp-Hyp-Gly-Flp-Hyp-
Gly;
(SEQ ID NO: 208)
Flp-Met-Gly-Flp-Hyp-Gly-Flp-Hyp-Gly-Flp-Hyp-Gly-Flp-Hyp-Gly-Flp-Hyp-Gly-Flp-Hyp-
Gly;
(SEQ ID NO: 209)
Flp-Hyp-Gly-Met-Hyp-Gly-Flp-Hyp-Gly-Flp-Hyp-Gly-Flp-Hyp-Gly-Flp-Hyp-Gly-Flp-Hyp-
Gly;
(SEQ ID NO: 210)
Flp-Hyp-Gly-Flp-Met-Gly-Flp-Hyp-Gly-Flp-Hyp-Gly-Flp-Hyp-Gly-Flp-Hyp-Gly-Flp-Hyp-
Gly;
(SEQ ID NO: 211)
Flp-Hyp-Gly-Flp-Hyp-Gly-Met-Hyp-Gly-Flp-Hyp-Gly-Flp-Hyp-Gly-Flp-Hyp-Gly-Flp-Hyp-
Gly;
(SEQ ID NO: 212)
Flp-Hyp-Gly-Flp-Hyp-Gly-Flp-Met-Gly-Flp-Hyp-Gly-Flp-Hyp-Gly-Flp-Hyp-Gly-Flp-Hyp-
Gly;
(SEQ ID NO: 213)
Flp-Hyp-Gly-Flp-Hyp-Gly-Flp-Hyp-Gly-Met-Hyp-Gly-Flp-Hyp-Gly-Flp-Hyp-Gly-Flp-Hyp-
Gly;
(SEQ ID NO: 214)
Flp-Hyp-Gly-Flp-Hyp-Gly-Flp-Hyp-Gly-Flp-Met-Gly-Flp-Hyp-Gly-Flp-Hyp-Gly-Flp-Hyp-
Gly;
(SEQ ID NO: 215)
Flp-Hyp-Gly-Flp-Hyp-Gly-Flp-Hyp-Gly-Flp-Hyp-Gly-Met-Hyp-Gly-Flp-Hyp-Gly-Flp-Hyp-
Gly;
(SEQ ID NO: 216)
Flp-Hyp-Gly-Flp-Hyp-Gly-Flp-Hyp-Gly-Flp-Hyp-Gly-Flp-Met-Gly-Flp-Hyp-Gly-Flp-Hyp-
Gly;
(SEQ ID NO: 217)
Flp-Hyp-Gly-Flp-Hyp-Gly-Flp-Hyp-Gly-Flp-Hyp-Gly-Flp-Hyp-Gly-Met-Hyp-Gly-Flp-Hyp-
Gly;
(SEQ ID NO: 218)
Flp-Hyp-Gly-Flp-Hyp-Gly-Flp-Hyp-Gly-Flp-Hyp-Gly-Flp-Hyp-Gly-Flp-Met-Gly-Flp-Hyp-
Gly;
(SEQ ID NO: 219)
Flp-Hyp-Gly-Flp-Hyp-Gly-Flp-Hyp-Gly-Flp-Hyp-Gly-Flp-Hyp-Gly-Flp-Hyp-Gly-Met-Hyp-
Gly;
(SEQ ID NO: 220)
Flp-Hyp-Gly-Flp-Hyp-Gly-Flp-Hyp-Gly-Flp-Hyp-Gly-Flp-Hyp-Gly-Flp-Hyp-Gly-Flp-Met-
Gly;
(SEQ ID NO: 221)
Met-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 222)
Pro-Met-Gly-Pro-Clp-Gly-Pro-Clp-Gly-Pro-Clp-Gly-Pro-Clp-Gly-Pro-Clp-Gly-Pro-Clp-Gly;
(SEQ ID NO: 223)
Pro-Clp-Gly-Met-Clp-Gly-Pro-Clp-Gly-Pro-Clp-Gly-Pro-Clp-Gly-Pro-Clp-Gly-Pro-Clp-Gly;
(SEQ ID NO: 224)
Pro-Clp-Gly-Pro-Met-Gly-Pro-Clp-Gly-Pro-Clp-Gly-Pro-Clp-Gly-Pro-Clp-Gly-Pro-Clp-Gly;
(SEQ ID NO: 225)
Pro-Clp-Gly-Pro-Clp-Gly-Met-Clp-Gly-Pro-Clp-Gly-Pro-Clp-Gly-Pro-Clp-Gly-Pro-Clp-Gly;
(SEQ ID NO: 226)
Pro-Clp-Gly-Pro-Clp-Gly-Pro-Clp-Gly-Met-Clp-Gly-Pro-Clp-Gly-Pro-Clp-Gly-Pro-Clp-Gly;
(SEQ ID NO: 227)
Pro-Clp-Gly-Pro-Clp-Gly-Pro-Clp-Gly-Pro-Met-Gly-Pro-Clp-Gly-Pro-Clp-Gly-Pro-Clp-Gly;
(SEQ ID NO: 228)
Pro-Clp-Gly-Pro-Clp-Gly-Pro-Clp-Gly-Pro-Clp-Gly-Met-Clp-Gly-Pro-Clp-Gly-Pro-Clp-Gly;
(SEQ ID NO: 229)
Pro-Clp-Gly-Pro-Clp-Gly-Pro-Clp-Gly-Pro-Clp-Gly-Pro-Met-Gly-Pro-Clp-Gly-Pro-Clp-Gly;
(SEQ ID NO: 230)
Pro-Clp-Gly-Pro-Clp-Gly-Pro-Clp-Gly-Pro-Clp-Gly-Pro-Clp-Gly-Met-Clp-Gly-Pro-Clp-Gly;
(SEQ ID NO: 231)
Pro-Clp-Gly-Pro-Clp-Gly-Pro-Clp-Gly-Pro-Clp-Gly-Pro-Clp-Gly-Pro-Met-Gly-Pro-Clp-Gly;
(SEQ ID NO: 232)
Pro-Clp-Gly-Pro-Clp-Gly-Pro-Clp-Gly-Pro-Clp-Gly-Pro-Clp-Gly-Pro-Clp-Gly-Met-Clp-Gly;
(SEQ ID NO: 233)
Pro-Clp-Gly-Pro-Clp-Gly-Pro-Clp-Gly-Pro-Clp-Gly-Pro-Clp-Gly-Pro-Clp-Gly-Pro-Met-Gly;
(SEQ ID NO: 234)
Met-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 235)
Pro-Met-Gly-Pro-Clp-Gly-Pro-Clp-Gly-Pro-Clp-Gly-Pro-Clp-Gly-Pro-Clp-Gly-Pro-Clp-Gly;
(SEQ ID NO: 236)
Pro-Clp-Gly-Met-Clp-Gly-Pro-Clp-Gly-Pro-Clp-Gly-Pro-Clp-Gly-Pro-Clp-Gly-Pro-Clp-Gly;
(SEQ ID NO: 237)
Pro-Clp-Gly-Pro-Met-Gly-Pro-Clp-Gly-Pro-Clp-Gly-Pro-Clp-Gly-Pro-Clp-Gly-Pro-Clp-Gly;
(SEQ ID NO: 238)
Pro-Clp-Gly-Pro-Clp-Gly-Met-Clp-Gly-Pro-Clp-Gly-Pro-Clp-Gly-Pro-Clp-Gly-Pro-Clp-Gly;
(SEQ ID NO: 239)
Pro-Clp-Gly-Pro-Clp-Gly-Pro-Met-Gly-Pro-Clp-Gly-Pro-Clp-Gly-Pro-Clp-Gly-Pro-Clp-Gly;
(SEQ ID NO: 240)
Pro-Clp-Gly-Pro-Clp-Gly-Pro-Clp-Gly-Met-Clp-Gly-Pro-Clp-Gly-Pro-Clp-Gly-Pro-Clp-Gly;
(SEQ ID NO: 241)
Pro-Clp-Gly-Pro-Clp-Gly-Pro-Clp-Gly-Pro-Met-Gly-Pro-Clp-Gly-Pro-Clp-Gly-Pro-Clp-Gly;
(SEQ ID NO: 242)
Pro-Clp-Gly-Pro-Clp-Gly-Pro-Clp-Gly-Pro-Clp-Gly-Met-Clp-Gly-Pro-Clp-Gly-Pro-Clp-Gly;
(SEQ ID NO: 243)
Pro-Clp-Gly-Pro-Clp-Gly-Pro-Clp-Gly-Pro-Clp-Gly-Pro-Met-Gly-Pro-Clp-Gly-Pro-Clp-Gly;
(SEQ ID NO: 244)
Pro-Clp-Gly-Pro-Clp-Gly-Pro-Clp-Gly-Pro-Clp-Gly-Pro-Clp-Gly-Met-Clp-Gly-Pro-Clp-Gly;
(SEQ ID NO: 245)
Pro-Clp-Gly-Pro-Clp-Gly-Pro-Clp-Gly-Pro-Clp-Gly-Pro-Clp-Gly-Pro-Met-Gly-Pro-Clp-Gly;
(SEQ ID NO: 246)
Pro-Clp-Gly-Pro-Clp-Gly-Pro-Clp-Gly-Pro-Clp-Gly-Pro-Clp-Gly-Pro-Clp-Gly-Met-Clp-Gly;
(SEQ ID NO: 247)
Pro-Clp-Gly-Pro-Clp-Gly-Pro-Clp-Gly-Pro-Clp-Gly-Pro-Clp-Gly-Pro-Clp-Gly-Pro-Met-Gly;
(SEQ ID NO: 248)
Met-Hyp-Gly-Clp-Hyp-Gly-Clp-Hyp-Gly-Clp-Hyp-Gly-Clp-Hyp-Gly-Clp-Hyp-Gly-Clp-
Hyp-Gly;
(SEQ ID NO: 249)
Clp-Met-Gly-Clp-Hyp-Gly-Clp-Hyp-Gly-Clp-Hyp-Gly-Clp-Hyp-Gly-Clp-Hyp-Gly-Clp-
Hyp-Gly;
(SEQ ID NO: 250)
Clp-Hyp-Gly-Met-Hyp-Gly-Clp-Hyp-Gly-Clp-Hyp-Gly-Clp-Hyp-Gly-Clp-Hyp-Gly-Clp-
Hyp-Gly;
(SEQ ID NO: 251)
Clp-Hyp-Gly-Clp-Met-Gly-Clp-Hyp-Gly-Clp-Hyp-Gly-Clp-Hyp-Gly-Clp-Hyp-Gly-Clp-
Hyp-Gly;
(SEQ ID NO: 252)
Clp-Hyp-Gly-Clp-Hyp-Gly-Met-Hyp-Gly-Clp-Hyp-Gly-Clp-Hyp-Gly-Clp-Hyp-Gly-Clp-
Hyp-Gly;
(SEQ ID NO: 253)
Clp-Hyp-Gly-Clp-Hyp-Gly-Clp-Met-Gly-Clp-Hyp-Gly-Clp-Hyp-Gly-Clp-Hyp-Gly-Clp-
Hyp-Gly;
(SEQ ID NO: 254)
Clp-Hyp-Gly-Clp-Hyp-Gly-Clp-Hyp-Gly-Met-Hyp-Gly-Clp-Hyp-Gly-Clp-Hyp-Gly-Clp-
Hyp-Gly;
(SEQ ID NO: 255)
Clp-Hyp-Gly-Clp-Hyp-Gly-Clp-Hyp-Gly-Clp-Met-Gly-Clp-Hyp-Gly-Clp-Hyp-Gly-Clp-
Hyp-Gly;
(SEQ ID NO: 256)
Clp-Hyp-Gly-Clp-Hyp-Gly-Clp-Hyp-Gly-Clp-Hyp-Gly-Met-Hyp-Gly-Clp-Hyp-Gly-Clp-
Hyp-Gly;
(SEQ ID NO: 257)
Clp-Hyp-Gly-Clp-Hyp-Gly-Clp-Hyp-Gly-Clp-Hyp-Gly-Clp-Met-Gly-Clp-Hyp-Gly-Clp-
Hyp-Gly;
(SEQ ID NO: 258)
Clp-Hyp-Gly-Clp-Hyp-Gly-Clp-Hyp-Gly-Clp-Hyp-Gly-Clp-Hyp-Gly-Met-Hyp-Gly-Clp-
Hyp-Gly;
(SEQ ID NO: 259)
Clp-Hyp-Gly-Clp-Hyp-Gly-Clp-Hyp-Gly-Clp-Hyp-Gly-Clp-Hyp-Gly-Clp-Met-Gly-Clp-
Hyp-Gly;
(SEQ ID NO: 260)
Clp-Hyp-Gly-Clp-Hyp-Gly-Clp-Hyp-Gly-Clp-Hyp-Gly-Clp-Hyp-Gly-Clp-Hyp-Gly-Met-
Hyp-Gly;
and
(SEQ ID NO: 261)
Clp-Hyp-Gly-Clp-Hyp-Gly-Clp-Hyp-Gly-Clp-Hyp-Gly-Clp-Hyp-Gly-Clp-Hyp-Gly-Clp-
Met-Gly.

In certain other embodiments of the invention, the collagen mimetic peptide may comprise, consist of or have an amino acid sequence that is or corresponds to a 21-mer of any one of SEQ ID NOs:1-9, in which at least one lysine (Lys) residue has been substituted for at least one of the proline residues in SEQ ID NO:1, at least one of the hydroxyproline residues in SEQ ID NOs:2-3 and 6, at least one of the fluoroproline residues in SEQ ID NOs:4-6, or at least one of the chloroproline residues in SEQ ID NOs:7-9, yielding, for example, the following sequences:

(SEQ ID NO: 262)
Pro-Pro-Gly-Pro-Pro-Gly-Lys-Pro-Gly-Pro-Pro-Gly-Pro-Pro-Gly-Pro-Pro-Gly-Pro-Pro-Gly;
(SEQ ID NO: 263)
Hyp-Pro-Gly-Hyp-Pro-Gly-Lys-Pro-Gly-Hyp-Pro-Gly-Hyp-Pro-Gly-Hyp-Pro-Gly-Hyp-Pro-
Gly;
(SEQ ID NO: 264)
Pro-Hyp-Gly-Pro-Hyp-Gly-Pro-Lys-Gly-Pro-Hyp-Gly-Pro-Hyp-Gly-Pro-Hyp-Gly-Pro-Hyp-
Gly;
(SEQ ID NO: 265)
Pro-Flp-Gly-Pro-Flp-Gly-Pro-Lys-Gly-Pro-Flp-Gly-Pro-Flp-Gly-Pro-Flp-Gly-Pro-Flp-Gly;
(SEQ ID NO: 266)
Pro-Clp-Gly-Pro-Clp-Gly-Pro-Lys-Gly-Pro-Clp-Gly-Pro-Clp-Gly-Pro-Clp-Gly-Pro-Clp-Gly;
(SEQ ID NO: 267)
Lys-Pro-Gly-Pro-Pro-Gly-Pro-Pro-Gly-Pro-Pro-Gly-Pro-Pro-Gly-Pro-Pro-Gly-Pro-Pro-Gly;
(SEQ ID NO: 268)
Pro-Lys-Gly-Pro-Pro-Gly-Pro-Pro-Gly-Pro-Pro-Gly-Pro-Pro-Gly-Pro-Pro-Gly-Pro-Pro-Gly;
(SEQ ID NO: 269)
Pro-Pro-Gly-Lys-Pro-Gly-Pro-Pro-Gly-Pro-Pro-Gly-Pro-Pro-Gly-Pro-Pro-Gly-Pro-Pro-Gly;
(SEQ ID NO: 270)
Pro-Pro-Gly-Pro-Lys-Gly-Pro-Pro-Gly-Pro-Pro-Gly-Pro-Pro-Gly-Pro-Pro-Gly-Pro-Pro-Gly;
(SEQ ID NO: 271)
Pro-Pro-Gly-Pro-Pro-Gly-Pro-Lys-Gly-Pro-Pro-Gly-Pro-Pro-Gly-Pro-Pro-Gly-Pro-Pro-Gly;
(SEQ ID NO: 272)
Pro-Pro-Gly-Pro-Pro-Gly-Pro-Pro-Gly-Lys-Pro-Gly-Pro-Pro-Gly-Pro-Pro-Gly-Pro-Pro-Gly;
(SEQ ID NO: 273)
Pro-Pro-Gly-Pro-Pro-Gly-Pro-Pro-Gly-Pro-Lys-Gly-Pro-Pro-Gly-Pro-Pro-Gly-Pro-Pro-Gly;
(SEQ ID NO: 274)
Pro-Pro-Gly-Pro-Pro-Gly-Pro-Pro-Gly-Pro-Pro-Gly-Lys-Pro-Gly-Pro-Pro-Gly-Pro-Pro-Gly;
(SEQ ID NO: 275)
Pro-Pro-Gly-Pro-Pro-Gly-Pro-Pro-Gly-Pro-Pro-Gly-Pro-Lys-Gly-Pro-Pro-Gly-Pro-Pro-Gly;
(SEQ ID NO: 276)
Pro-Pro-Gly-Pro-Pro-Gly-Pro-Pro-Gly-Pro-Pro-Gly-Pro-Pro-Gly-Lys-Pro-Gly-Pro-Pro-Gly;
(SEQ ID NO: 277)
Pro-Pro-Gly-Pro-Pro-Gly-Pro-Pro-Gly-Pro-Pro-Gly-Pro-Pro-Gly-Pro-Lys-Gly-Pro-Pro-Gly;
(SEQ ID NO: 278)
Pro-Pro-Gly-Pro-Pro-Gly-Pro-Pro-Gly-Pro-Pro-Gly-Pro-Pro-Gly-Pro-Pro-Gly-Lys-Pro-Gly;
(SEQ ID NO: 279)
Pro-Pro-Gly-Pro-Pro-Gly-Pro-Pro-Gly-Pro-Pro-Gly-Pro-Pro-Gly-Pro-Pro-Gly-Pro-Lys-Gly;
(SEQ ID NO: 280)
Lys-Pro-Gly-Hyp-Pro-Gly-Hyp-Pro-Gly-Hyp-Pro-Gly-Hyp-Pro-Gly-Hyp-Pro-Gly-Hyp-Pro-
Gly;
(SEQ ID NO: 281)
Hyp-Lys-Gly-Hyp-Pro-Gly-Hyp-Pro-Gly-Hyp-Pro-Gly-Hyp-Pro-Gly-Hyp-Pro-Gly-Hyp-
Pro-Gly;
(SEQ ID NO: 282)
Hyp-Pro-Gly-Lys-Pro-Gly-Hyp-Pro-Gly-Hyp-Pro-Gly-Hyp-Pro-Gly-Hyp-Pro-Gly-Hyp-Pro-
Gly;
(SEQ ID NO: 283)
Hyp-Pro-Gly-Hyp-Lys-Gly-Hyp-Pro-Gly-Hyp-Pro-Gly-Hyp-Pro-Gly-Hyp-Pro-Gly-Hyp-
Pro-Gly;
(SEQ ID NO: 284)
Hyp-Pro-Gly-Hyp-Pro-Gly-Hyp-Lys-Gly-Hyp-Pro-Gly-Hyp-Pro-Gly-Hyp-Pro-Gly-Hyp-
Pro-Gly;
(SEQ ID NO: 285)
Hyp-Pro-Gly-Hyp-Pro-Gly-Hyp-Pro-Gly-Lys-Pro-Gly-Hyp-Pro-Gly-Hyp-Pro-Gly-Hyp-Pro-
Gly;
(SEQ ID NO: 286)
Hyp-Pro-Gly-Hyp-Pro-Gly-Hyp-Pro-Gly-Hyp-Lys-Gly-Hyp-Pro-Gly-Hyp-Pro-Gly-Hyp-
Pro-Gly;
(SEQ ID NO: 287)
Hyp-Pro-Gly-Hyp-Pro-Gly-Hyp-Pro-Gly-Hyp-Pro-Gly-Lys-Pro-Gly-Hyp-Pro-Gly-Hyp-Pro-
Gly;
(SEQ ID NO: 288)
Hyp-Pro-Gly-Hyp-Pro-Gly-Hyp-Pro-Gly-Hyp-Pro-Gly-Hyp-Lys-Gly-Hyp-Pro-Gly-Hyp-
Pro-Gly;
(SEQ ID NO: 289)
Hyp-Pro-Gly-Hyp-Pro-Gly-Hyp-Pro-Gly-Hyp-Pro-Gly-Hyp-Pro-Gly-Lys-Pro-Gly-Hyp-Pro-
Gly;
(SEQ ID NO: 290)
Hyp-Pro-Gly-Hyp-Pro-Gly-Hyp-Pro-Gly-Hyp-Pro-Gly-Hyp-Pro-Gly-Hyp-Lys-Gly-Hyp-
Pro-Gly;
(SEQ ID NO: 291)
Hyp-Pro-Gly-Hyp-Pro-Gly-Hyp-Pro-Gly-Hyp-Pro-Gly-Hyp-Pro-Gly-Hyp-Pro-Gly-Lys-Pro-
Gly;
(SEQ ID NO: 292)
Hyp-Pro-Gly-Hyp-Pro-Gly-Hyp-Pro-Gly-Hyp-Pro-Gly-Hyp-Pro-Gly-Hyp-Pro-Gly-Hyp-
Lys-Gly;
(SEQ ID NO: 293)
Lys-Hyp-Gly-Pro-Hyp-Gly-Pro-Hyp-Gly-Pro-Hyp-Gly-Pro-Hyp-Gly-Pro-Hyp-Gly-Pro-
Hyp-Gly;
(SEQ ID NO: 294)
Pro-Lys-Gly-Pro-Hyp-Gly-Pro-Hyp-Gly-Pro-Hyp-Gly-Pro-Hyp-Gly-Pro-Hyp-Gly-Pro-Hyp-
Gly;
(SEQ ID NO: 295)
Pro-Hyp-Gly-Lys-Hyp-Gly-Pro-Hyp-Gly-Pro-Hyp-Gly-Pro-Hyp-Gly-Pro-Hyp-Gly-Pro-
Hyp-Gly;
(SEQ ID NO: 296)
Pro-Hyp-Gly-Pro-Lys-Gly-Pro-Hyp-Gly-Pro-Hyp-Gly-Pro-Hyp-Gly-Pro-Hyp-Gly-Pro-Hyp-
Gly;
(SEQ ID NO: 297)
Pro-Hyp-Gly-Pro-Hyp-Gly-Lys-Hyp-Gly-Pro-Hyp-Gly-Pro-Hyp-Gly-Pro-Hyp-Gly-Pro-
Hyp-Gly;
(SEQ ID NO: 298)
Pro-Hyp-Gly-Pro-Hyp-Gly-Pro-Hyp-Gly-Lys-Hyp-Gly-Pro-Hyp-Gly-Pro-Hyp-Gly-Pro-
Hyp-Gly;
(SEQ ID NO: 299)
Pro-Hyp-Gly-Pro-Hyp-Gly-Pro-Hyp-Gly-Pro-Lys-Gly-Pro-Hyp-Gly-Pro-Hyp-Gly-Pro-Hyp-
Gly;
(SEQ ID NO: 300)
Pro-Hyp-Gly-Pro-Hyp-Gly-Pro-Hyp-Gly-Pro-Hyp-Gly-Lys-Hyp-Gly-Pro-Hyp-Gly-Pro-
Hyp-Gly;
(SEQ ID NO: 301)
Pro-Hyp-Gly-Pro-Hyp-Gly-Pro-Hyp-Gly-Pro-Hyp-Gly-Pro-Lys-Gly-Pro-Hyp-Gly-Pro-Hyp-
Gly;
(SEQ ID NO: 302)
Pro-Hyp-Gly-Pro-Hyp-Gly-Pro-Hyp-Gly-Pro-Hyp-Gly-Pro-Hyp-Gly-Lys-Hyp-Gly-Pro-
Hyp-Gly;
(SEQ ID NO: 303)
Pro-Hyp-Gly-Pro-Hyp-Gly-Pro-Hyp-Gly-Pro-Hyp-Gly-Pro-Hyp-Gly-Pro-Lys-Gly-Pro-Hyp-
Gly;
(SEQ ID NO: 304)
Pro-Hyp-Gly-Pro-Hyp-Gly-Pro-Hyp-Gly-Pro-Hyp-Gly-Pro-Hyp-Gly-Pro-Hyp-Gly-Lys-
Hyp-Gly;
(SEQ ID NO: 305)
Pro-Hyp-Gly-Pro-Hyp-Gly-Pro-Hyp-Gly-Pro-Hyp-Gly-Pro-Hyp-Gly-Pro-Hyp-Gly-Pro-Lys-
Gly;
(SEQ ID NO: 306)
Lys-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 307)
Pro-Lys-Gly-Pro-Flp-Gly-Pro-Flp-Gly-Pro-Flp-Gly-Pro-Flp-Gly-Pro-Flp-Gly-Pro-Flp-Gly;
(SEQ ID NO: 308)
Pro-Flp-Gly-Lys-Flp-Gly-Pro-Flp-Gly-Pro-Flp-Gly-Pro-Flp-Gly-Pro-Flp-Gly-Pro-Flp-Gly;
(SEQ ID NO: 309)
Pro-Flp-Gly-Pro-Lys-Gly-Pro-Flp-Gly-Pro-Flp-Gly-Pro-Flp-Gly-Pro-Flp-Gly-Pro-Flp-Gly;
(SEQ ID NO: 310)
Pro-Flp-Gly-Pro-Flp-Gly-Lys-Flp-Gly-Pro-Flp-Gly-Pro-Flp-Gly-Pro-Flp-Gly-Pro-Flp-Gly;
(SEQ ID NO: 311)
Pro-Flp-Gly-Pro-Flp-Gly-Pro-Flp-Gly-Lys-Flp-Gly-Pro-Flp-Gly-Pro-Flp-Gly-Pro-Flp-Gly;
(SEQ ID NO: 312)
Pro-Flp-Gly-Pro-Flp-Gly-Pro-Flp-Gly-Pro-Lys-Gly-Pro-Flp-Gly-Pro-Flp-Gly-Pro-Flp-Gly;
(SEQ ID NO: 313)
Pro-Flp-Gly-Pro-Flp-Gly-Pro-Flp-Gly-Pro-Flp-Gly-Lys-Flp-Gly-Pro-Flp-Gly-Pro-Flp-Gly;
(SEQ ID NO: 314)
Pro-Flp-Gly-Pro-Flp-Gly-Pro-Flp-Gly-Pro-Flp-Gly-Pro-Lys-Gly-Pro-Flp-Gly-Pro-Flp-Gly;
(SEQ ID NO: 315)
Pro-Flp-Gly-Pro-Flp-Gly-Pro-Flp-Gly-Pro-Flp-Gly-Pro-Flp-Gly-Lys-Flp-Gly-Pro-Flp-Gly;
(SEQ ID NO: 316)
Pro-Flp-Gly-Pro-Flp-Gly-Pro-Flp-Gly-Pro-Flp-Gly-Pro-Flp-Gly-Pro-Lys-Gly-Pro-Flp-Gly;
(SEQ ID NO: 317)
Pro-Flp-Gly-Pro-Flp-Gly-Pro-Flp-Gly-Pro-Flp-Gly-Pro-Flp-Gly-Pro-Flp-Gly-Lys-Flp-Gly;
(SEQ ID NO: 318)
Pro-Flp-Gly-Pro-Flp-Gly-Pro-Flp-Gly-Pro-Flp-Gly-Pro-Flp-Gly-Pro-Flp-Gly-Pro-Lys-Gly;
(SEQ ID NO: 319)
Lys-Flp-Gly-Pro-Flp-Gly-Pro-Flp-Gly-Pro-Flp-Gly-Pro-Flp-Gly-Pro-Flp-Gly-Pro-Flp-Gly;
(SEQ ID NO: 320)
Pro-Lys-Gly-Pro-Flp-Gly-Pro-Flp-Gly-Pro-Flp-Gly-Pro-Flp-Gly-Pro-Flp-Gly-Pro-Flp-Gly;
(SEQ ID NO: 321)
Pro-Flp-Gly-Lys-Flp-Gly-Pro-Flp-Gly-Pro-Flp-Gly-Pro-Flp-Gly-Pro-Flp-Gly-Pro-Flp-Gly;
(SEQ ID NO: 322)
Pro-Flp-Gly-Pro-Lys-Gly-Pro-Flp-Gly-Pro-Flp-Gly-Pro-Flp-Gly-Pro-Flp-Gly-Pro-Flp-Gly;
(SEQ ID NO: 323)
Pro-Flp-Gly-Pro-Flp-Gly-Lys-Flp-Gly-Pro-Flp-Gly-Pro-Flp-Gly-Pro-Flp-Gly-Pro-Flp-Gly;
(SEQ ID NO: 324)
Pro-Flp-Gly-Pro-Flp-Gly-Pro-Lys-Gly-Pro-Flp-Gly-Pro-Flp-Gly-Pro-Flp-Gly-Pro-Flp-Gly;
(SEQ ID NO: 325)
Pro-Flp-Gly-Pro-Flp-Gly-Pro-Flp-Gly-Lys-Flp-Gly-Pro-Flp-Gly-Pro-Flp-Gly-Pro-Flp-Gly;
(SEQ ID NO: 326)
Pro-Flp-Gly-Pro-Flp-Gly-Pro-Flp-Gly-Pro-Lys-Gly-Pro-Flp-Gly-Pro-Flp-Gly-Pro-Flp-Gly;
(SEQ ID NO: 327)
Pro-Flp-Gly-Pro-Flp-Gly-Pro-Flp-Gly-Pro-Flp-Gly-Lys-Flp-Gly-Pro-Flp-Gly-Pro-Flp-Gly;
(SEQ ID NO: 328)
Pro-Flp-Gly-Pro-Flp-Gly-Pro-Flp-Gly-Pro-Flp-Gly-Pro-Lys-Gly-Pro-Flp-Gly-Pro-Flp-Gly;
(SEQ ID NO: 329)
Pro-Flp-Gly-Pro-Flp-Gly-Pro-Flp-Gly-Pro-Flp-Gly-Pro-Flp-Gly-Lys-Flp-Gly-Pro-Flp-Gly;
(SEQ ID NO: 330)
Pro-Flp-Gly-Pro-Flp-Gly-Pro-Flp-Gly-Pro-Flp-Gly-Pro-Flp-Gly-Pro-Lys-Gly-Pro-Flp-Gly;
(SEQ ID NO: 331)
Pro-Flp-Gly-Pro-Flp-Gly-Pro-Flp-Gly-Pro-Flp-Gly-Pro-Flp-Gly-Pro-Flp-Gly-Lys-Flp-Gly;
(SEQ ID NO: 332)
Pro-Flp-Gly-Pro-Flp-Gly-Pro-Flp-Gly-Pro-Flp-Gly-Pro-Flp-Gly-Pro-Flp-Gly-Pro-Lys-Gly;
(SEQ ID NO: 333)
Lys-Hyp-Gly-Flp-Hyp-Gly-Flp-Hyp-Gly-Flp-Hyp-Gly-Flp-Hyp-Gly-Flp-Hyp-Gly-Flp-Hyp-
Gly;
(SEQ ID NO: 334)
Flp-Lys-Gly-Flp-Hyp-Gly-Flp-Hyp-Gly-Flp-Hyp-Gly-Flp-Hyp-Gly-Flp-Hyp-Gly-Flp-Hyp-
Gly;
(SEQ ID NO: 335)
Flp-Hyp-Gly-Lys-Hyp-Gly-Flp-Hyp-Gly-Flp-Hyp-Gly-Flp-Hyp-Gly-Flp-Hyp-Gly-Flp-Hyp-
Gly;
(SEQ ID NO: 336)
Flp-Hyp-Gly-Flp-Lys-Gly-Flp-Hyp-Gly-Flp-Hyp-Gly-Flp-Hyp-Gly-Flp-Hyp-Gly-Flp-Hyp-
Gly;
(SEQ ID NO: 337)
Flp-Hyp-Gly-Flp-Hyp-Gly-Lys-Hyp-Gly-Flp-Hyp-Gly-Flp-Hyp-Gly-Flp-Hyp-Gly-Flp-Hyp-
Gly;
(SEQ ID NO: 338)
Flp-Hyp-Gly-Flp-Hyp-Gly-Flp-Lys-Gly-Flp-Hyp-Gly-Flp-Hyp-Gly-Flp-Hyp-Gly-Flp-Hyp-
Gly;
(SEQ ID NO: 339)
Flp-Hyp-Gly-Flp-Hyp-Gly-Flp-Hyp-Gly-Lys-Hyp-Gly-Flp-Hyp-Gly-Flp-Hyp-Gly-Flp-Hyp-
Gly;
(SEQ ID NO: 340)
Flp-Hyp-Gly-Flp-Hyp-Gly-Flp-Hyp-Gly-Flp-Lys-Gly-Flp-Hyp-Gly-Flp-Hyp-Gly-Flp-Hyp-
Gly;
(SEQ ID NO: 341)
Flp-Hyp-Gly-Flp-Hyp-Gly-Flp-Hyp-Gly-Flp-Hyp-Gly-Lys-Hyp-Gly-Flp-Hyp-Gly-Flp-Hyp-
Gly;
(SEQ ID NO: 342)
Flp-Hyp-Gly-Flp-Hyp-Gly-Flp-Hyp-Gly-Flp-Hyp-Gly-Flp-Lys-Gly-Flp-Hyp-Gly-Flp-Hyp-
Gly;
(SEQ ID NO: 343)
Flp-Hyp-Gly-Flp-Hyp-Gly-Flp-Hyp-Gly-Flp-Hyp-Gly-Flp-Hyp-Gly-Lys-Hyp-Gly-Flp-Hyp-
Gly;
(SEQ ID NO: 344)
Flp-Hyp-Gly-Flp-Hyp-Gly-Flp-Hyp-Gly-Flp-Hyp-Gly-Flp-Hyp-Gly-Flp-Lys-Gly-Flp-Hyp-
Gly;
(SEQ ID NO: 345)
Flp-Hyp-Gly-Flp-Hyp-Gly-Flp-Hyp-Gly-Flp-Hyp-Gly-Flp-Hyp-Gly-Flp-Hyp-Gly-Lys-Hyp-
Gly;
(SEQ ID NO: 346)
Flp-Hyp-Gly-Flp-Hyp-Gly-Flp-Hyp-Gly-Flp-Hyp-Gly-Flp-Hyp-Gly-Flp-Hyp-Gly-Flp-Lys-
Gly;
(SEQ ID NO: 347)
Lys-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 348)
Pro-Lys-Gly-Pro-Clp-Gly-Pro-Clp-Gly-Pro-Clp-Gly-Pro-Clp-Gly-Pro-Clp-Gly-Pro-Clp-Gly;
(SEQ ID NO: 349)
Pro-Clp-Gly-Lys-Clp-Gly-Pro-Clp-Gly-Pro-Clp-Gly-Pro-Clp-Gly-Pro-Clp-Gly-Pro-Clp-Gly;
(SEQ ID NO: 350)
Pro-Clp-Gly-Pro-Lys-Gly-Pro-Clp-Gly-Pro-Clp-Gly-Pro-Clp-Gly-Pro-Clp-Gly-Pro-Clp-Gly;
(SEQ ID NO: 351)
Pro-Clp-Gly-Pro-Clp-Gly-Lys-Clp-Gly-Pro-Clp-Gly-Pro-Clp-Gly-Pro-Clp-Gly-Pro-Clp-Gly;
(SEQ ID NO: 352)
Pro-Clp-Gly-Pro-Clp-Gly-Pro-Clp-Gly-Lys-Clp-Gly-Pro-Clp-Gly-Pro-Clp-Gly-Pro-Clp-Gly;
(SEQ ID NO: 353)
Pro-Clp-Gly-Pro-Clp-Gly-Pro-Clp-Gly-Pro-Lys-Gly-Pro-Clp-Gly-Pro-Clp-Gly-Pro-Clp-Gly;
(SEQ ID NO: 354)
Pro-Clp-Gly-Pro-Clp-Gly-Pro-Clp-Gly-Pro-Clp-Gly-Lys-Clp-Gly-Pro-Clp-Gly-Pro-Clp-Gly;
(SEQ ID NO: 355)
Pro-Clp-Gly-Pro-Clp-Gly-Pro-Clp-Gly-Pro-Clp-Gly-Pro-Lys-Gly-Pro-Clp-Gly-Pro-Clp-Gly;
(SEQ ID NO: 356)
Pro-Clp-Gly-Pro-Clp-Gly-Pro-Clp-Gly-Pro-Clp-Gly-Pro-Clp-Gly-Lys-Clp-Gly-Pro-Clp-Gly;
(SEQ ID NO: 357)
Pro-Clp-Gly-Pro-Clp-Gly-Pro-Clp-Gly-Pro-Clp-Gly-Pro-Clp-Gly-Pro-Lys-Gly-Pro-Clp-Gly;
(SEQ ID NO: 358)
Pro-Clp-Gly-Pro-Clp-Gly-Pro-Clp-Gly-Pro-Clp-Gly-Pro-Clp-Gly-Pro-Clp-Gly-Lys-Clp-Gly;
(SEQ ID NO: 359)
Pro-Clp-Gly-Pro-Clp-Gly-Pro-Clp-Gly-Pro-Clp-Gly-Pro-Clp-Gly-Pro-Clp-Gly-Pro-Lys-Gly;
(SEQ ID NO: 360)
Lys-Clp-Gly-Pro-Clp-Gly-Pro-Clp-Gly-Pro-Clp-Gly-Pro-Clp-Gly-Pro-Clp-Gly-Pro-Clp-Gly;
(SEQ ID NO: 361)
Pro-Lys-Gly-Pro-Clp-Gly-Pro-Clp-Gly-Pro-Clp-Gly-Pro-Clp-Gly-Pro-Clp-Gly-Pro-Clp-Gly;
(SEQ ID NO: 362)
Pro-Clp-Gly-Lys-Clp-Gly-Pro-Clp-Gly-Pro-Clp-Gly-Pro-Clp-Gly-Pro-Clp-Gly-Pro-Clp-Gly;
(SEQ ID NO: 363)
Pro-Clp-Gly-Pro-Lys-Gly-Pro-Clp-Gly-Pro-Clp-Gly-Pro-Clp-Gly-Pro-Clp-Gly-Pro-Clp-Gly;
(SEQ ID NO: 364)
Pro-Clp-Gly-Pro-Clp-Gly-Lys-Clp-Gly-Pro-Clp-Gly-Pro-Clp-Gly-Pro-Clp-Gly-Pro-Clp-Gly;
(SEQ ID NO: 365)
Pro-Clp-Gly-Pro-Clp-Gly-Pro-Lys-Gly-Pro-Clp-Gly-Pro-Clp-Gly-Pro-Clp-Gly-Pro-Clp-Gly;
(SEQ ID NO: 366)
Pro-Clp-Gly-Pro-Clp-Gly-Pro-Clp-Gly-Lys-Clp-Gly-Pro-Clp-Gly-Pro-Clp-Gly-Pro-Clp-Gly;
(SEQ ID NO: 367)
Pro-Clp-Gly-Pro-Clp-Gly-Pro-Clp-Gly-Pro-Lys-Gly-Pro-Clp-Gly-Pro-Clp-Gly-Pro-Clp-Gly;
(SEQ ID NO: 368)
Pro-Clp-Gly-Pro-Clp-Gly-Pro-Clp-Gly-Pro-Clp-Gly-Lys-Clp-Gly-Pro-Clp-Gly-Pro-Clp-Gly;
(SEQ ID NO: 369)
Pro-Clp-Gly-Pro-Clp-Gly-Pro-Clp-Gly-Pro-Clp-Gly-Pro-Lys-Gly-Pro-Clp-Gly-Pro-Clp-Gly;
(SEQ ID NO: 370)
Pro-Clp-Gly-Pro-Clp-Gly-Pro-Clp-Gly-Pro-Clp-Gly-Pro-Clp-Gly-Lys-Clp-Gly-Pro-Clp-Gly;
(SEQ ID NO: 371)
Pro-Clp-Gly-Pro-Clp-Gly-Pro-Clp-Gly-Pro-Clp-Gly-Pro-Clp-Gly-Pro-Lys-Gly-Pro-Clp-Gly;
(SEQ ID NO: 372)
Pro-Clp-Gly-Pro-Clp-Gly-Pro-Clp-Gly-Pro-Clp-Gly-Pro-Clp-Gly-Pro-Clp-Gly-Lys-Clp-Gly;
(SEQ ID NO: 373)
Pro-Clp-Gly-Pro-Clp-Gly-Pro-Clp-Gly-Pro-Clp-Gly-Pro-Clp-Gly-Pro-Clp-Gly-Pro-Lys-Gly;
(SEQ ID NO: 374)
Lys-Hyp-Gly-Clp-Hyp-Gly-Clp-Hyp-Gly-Clp-Hyp-Gly-Clp-Hyp-Gly-Clp-Hyp-Gly-Clp-
Hyp-Gly;
(SEQ ID NO: 375)
Clp-Lys-Gly-Clp-Hyp-Gly-Clp-Hyp-Gly-Clp-Hyp-Gly-Clp-Hyp-Gly-Clp-Hyp-Gly-Clp-
Hyp-Gly;
(SEQ ID NO: 376)
Clp-Hyp-Gly-Lys-Hyp-Gly-Clp-Hyp-Gly-Clp-Hyp-Gly-Clp-Hyp-Gly-Clp-Hyp-Gly-Clp-
Hyp-Gly;
(SEQ ID NO: 377)
Clp-Hyp-Gly-Clp-Lys-Gly-Clp-Hyp-Gly-Clp-Hyp-Gly-Clp-Hyp-Gly-Clp-Hyp-Gly-Clp-
Hyp-Gly;
(SEQ ID NO: 378)
Clp-Hyp-Gly-Clp-Hyp-Gly-Lys-Hyp-Gly-Clp-Hyp-Gly-Clp-Hyp-Gly-Clp-Hyp-Gly-Clp-
Hyp-Gly;
(SEQ ID NO: 379)
Clp-Hyp-Gly-Clp-Hyp-Gly-Clp-Lys-Gly-Clp-Hyp-Gly-Clp-Hyp-Gly-Clp-Hyp-Gly-Clp-
Hyp-Gly;
(SEQ ID NO: 380)
Clp-Hyp-Gly-Clp-Hyp-Gly-Clp-Hyp-Gly-Lys-Hyp-Gly-Clp-Hyp-Gly-Clp-Hyp-Gly-Clp-
Hyp-Gly;
(SEQ ID NO: 381)
Clp-Hyp-Gly-Clp-Hyp-Gly-Clp-Hyp-Gly-Clp-Lys-Gly-Clp-Hyp-Gly-Clp-Hyp-Gly-Clp-
Hyp-Gly;
(SEQ ID NO: 382)
Clp-Hyp-Gly-Clp-Hyp-Gly-Clp-Hyp-Gly-Clp-Hyp-Gly-Lys-Hyp-Gly-Clp-Hyp-Gly-Clp-
Hyp-Gly;
(SEQ ID NO: 383)
Clp-Hyp-Gly-Clp-Hyp-Gly-Clp-Hyp-Gly-Clp-Hyp-Gly-Clp-Lys-Gly-Clp-Hyp-Gly-Clp-
Hyp-Gly;
(SEQ ID NO: 384)
Clp-Hyp-Gly-Clp-Hyp-Gly-Clp-Hyp-Gly-Clp-Hyp-Gly-Clp-Hyp-Gly-Lys-Hyp-Gly-Clp-
Hyp-Gly;
(SEQ ID NO: 385)
Clp-Hyp-Gly-Clp-Hyp-Gly-Clp-Hyp-Gly-Clp-Hyp-Gly-Clp-Hyp-Gly-Clp-Lys-Gly-Clp-
Hyp-Gly;
(SEQ ID NO: 386)
Clp-Hyp-Gly-Clp-Hyp-Gly-Clp-Hyp-Gly-Clp-Hyp-Gly-Clp-Hyp-Gly-Clp-Hyp-Gly-Lys-
Hyp-Gly;
and
(SEQ ID NO: 387)
Clp-Hyp-Gly-Clp-Hyp-Gly-Clp-Hyp-Gly-Clp-Hyp-Gly-Clp-Hyp-Gly-Clp-Hyp-Gly-Clp-
Lys-Gly.

Another suitable CMP for use according to the invention is a CMP having or comprising the sequence Hyp-Flp-Gly-Hyp-Flp-Gly-Hyp-Flp-Gly-Hyp-Flp-Gly-Hyp-Flp-Gly-Flp-Gly-Hyp-Flp-Gly (SEQ ID NO:388).

Other suitable CMPs for use according to the invention is a CMP having or comprising the sequence Gly₃-(Pro-Hyp-Gly)₆ (SEQ ID NO:397), Gly₃-(Pro-Flp-Gly)₆ (SEQ ID NO:398), Gly₃-(Pro-Hyp-Gly)₇ (SEQ ID NO:399), Gly₃-(Pro-Flp-Gly)₇ (SEQ ID NO:400), Gly₃-(Pro-Hyp-Gly)₈ (SEQ ID NO:401), Gly₃-(Pro-Flp-Gly)₈ (SEQ ID NO:402), Gly₃-(Pro-Hyp-Gly)₉ (SEQ ID NO:403), Gly₃-(Pro-Flp-Gly)₉ (SEQ ID NO:404), (Pro-Hyp-Gly)₆-Tyr (SEQ ID NO:405), (Pro-Flp-Gly)₆-Tyr (SEQ ID NO:406), (Pro-Hyp-Gly)₇-Tyr (SEQ ID NO:407), (Pro-Flp-Gly)₇-Tyr (SEQ ID NO:408), (Pro-Hyp-Gly)₈-Tyr (SEQ ID NO:409), (Pro-Flp-Gly)₈-Tyr (SEQ ID NO:410), Cys-(Pro-Hyp-Gly)₃ (SEQ ID NO:411), Cys-(Pro-Flp-Gly)₃ (SEQ ID NO:412), Cys-(Pro-Hyp-Gly)₅ (SEQ ID NO:413), Cys-(Pro-Flp-Gly)₅ (SEQ ID NO:414), Cys-(Pro-Hyp-Gly)₇ (SEQ ID NO:415), and Cys-(Pro-Flp-Gly)₇ (SEQ ID NO:416), and other analogous CMPs which may be suitable for use as agents for modification of collagen in vitro and in vivo for use in therapeutic and/or diagnostic methods (see, e.g., U.S. Pat. Nos. 8,283,414, 8,883,964, 10,632,168, and 11,389,513, the disclosures of all of which are incorporated herein by reference in their entireties).

In certain other embodiments of the invention, the collagen mimetic peptide comprises, consists essentially of or consists of an amino acid sequence that is or corresponds to a 21-mer comprising seven repeats of a three amino acid sequence in which Ala has been substituted for proline₂ in SEQ ID NO:1, yielding a sequence of seven repeats of proline-alanine-glycine ((Pro-Ala-Gly)₇), i.e., an amino acid sequence of: Pro-Ala-Gly-Pro-Ala-Gly-Pro-Ala-Gly-Pro-Ala-Gly-Pro-Ala-Gly-Pro-Ala-Gly-Pro-Ala-Gly (SEQ ID NO:418). In other embodiments, the collagen mimetic peptide comprises, consists essentially of or consists of an amino acid sequence that is or corresponds to a 21-mer comprising seven repeats of a three amino acid sequence in which either Hyp or Flp has been substituted for proline₁ in SEQ ID NO:418, yielding a sequence of seven repeats of proline-alanine-glycine ((Pro-Ala-Gly)₇), i.e., amino acid sequences of: Hyp-Ala-Gly-Hyp-Ala-Gly-Hyp-Ala-Gly-Hyp-Ala-Gly-Hyp-Ala-Gly-Hyp-Ala-Gly-Hyp-Ala-Gly (SEQ ID NO:419) and Flp-Ala-Gly-Flp-Ala-Gly-Flp-Ala-Gly-Flp-Ala-Gly-Flp-Ala-Gly-Flp-Ala-Gly-Flp-Ala-Gly (SEQ ID NO:420).

Other suitable CMPs for use according to the invention is a CMP having or comprising the sequence (Pro-Gly-Glu)₇ (SEQ ID NO:421), (Pro-Gly-Gln)₇ (SEQ ID NO:422), (Pro-Gly-Pro)₇ (SEQ ID NO:423), (Hyp-Gly-Glu)₇ (SEQ ID NO:424), (Hyp-Gly-Gln)₇ (SEQ ID NO:425), (Flp-Gly-Glu)₇ (SEQ ID NO:426), (Flp-Gly-Gln)₇ (SEQ ID NO:427), (Hyp-Gly-Pro)₇ (SEQ ID NO:428), (Hyp-Gly-Gln)₇ (SEQ ID NO:429), (Hyp-Gly-Glu)₇ (SEQ ID NO:430), (Pro-Gly-Asp)₇ (SEQ ID NO:431), (Pro-Gly-Asn)₇ (SEQ ID NO:432), (Lys-Gly-Gln)₇ (SEQ ID NO:433), (Lys-Gly-Glu)₇ (SEQ ID NO:434), (Hyp-Ala-Gly)₇ (SEQ ID NO:435), (Flp-Ala-Gly)₇ (SEQ ID NO:436), (Hyp-Gly-Glu)₇ (SEQ ID NO:437), (Hyp-Gly-Gln)₇ (SEQ ID NO:438), (Flp-Gly-Glu)₇ (SEQ ID NO:439), (Flp-Gly-Gln)₇ (SEQ ID NO:440), (Hyp-Gly-Asp)₇ (SEQ ID NO:441), (Hyp-Gly-Asn)₇ (SEQ ID NO:442), (Flp-Gly-Asp)₇ (SEQ ID NO:443), (Flp-Gly-Asn)₇ (SEQ ID NO:444), (Hyp-Gly-Pro)₇ (SEQ ID NO:445), (Flp-Gly-Pro)₇ (SEQ ID NO:446), (Pro-Gly-Hyp)₇ (SEQ ID NO:447), (Flp-Gly-Hyp)₇ (SEQ ID NO:448), (Hyp-Gly-Hyp)₇ (SEQ ID NO:449), (Hyp-Gly-Flp)₇ (SEQ ID NO:450), (Pro-Gly-Flp)₇ (SEQ ID NO:451), (Flp-Gly-Flp)₇ (SEQ ID NO:452), and other analogous CMPs comprising one or more additional or substituted amino acids including one or more cysteine residues, one or more methionine residues and/or one or more lysine residues, inserted or substituted into locations in the foregoing CMP sequences according to the locations set forth for cysteine in SEQ ID Nos:10-135, for methionine in SEQ ID Nos:136-251, and for lysine in SEQ ID Nos:262-387.

In certain other embodiments, any of the foregoing CMPs may optionally have one or more cysteine residues, one or more methionine residues, and/or one or more lysine residues, attached to either the N-terminus or the C-terminus, or to both termini, of the amino acid sequence. Non-limiting examples of such CMPs include:
SEQ ID NO:1 with a cysteine residue attached at the N-terminus, i.e., Cys-((Pro-Pro-Gly)₇) (SEQ ID NO:453), at the C-terminus, i.e., (Pro-Pro-Gly)₇-Cys (SEQ ID NO:454), or at both termini, i.e., Cys-((Pro-Pro-Gly)₇)-Cys (SEQ ID NO:455);
SEQ ID NO:6 with a cysteine residue attached at the N-terminus, i.e., Cys-((Flp-Hyp-Gly)₇) (SEQ ID NO:456), at the C-terminus, i.e., (Flp-Hyp-Gly)₇-Cys (SEQ ID NO:457), or at both termini, i.e., Cys-((Flp-Hyp-Gly)₇)-Cys (SEQ ID NO:458);
SEQ ID NO:1 with a methionine residue attached at the N-terminus, i.e., Met-((Pro-Pro-Gly)₇) (SEQ ID NO:459), at the C-terminus, i.e., (Pro-Pro-Gly)₇-Met (SEQ ID NO:460), or at both termini, i.e., Met-((Pro-Pro-Gly)₇)-Met (SEQ ID NO:461);
SEQ ID NO:6 with a methionine residue attached at the N-terminus, i.e., Met-((Flp-Hyp-Gly)₇) (SEQ ID NO:462), at the C-terminus, i.e., (Flp-Hyp-Gly)₇-Met (SEQ ID NO:463), or at both termini, i.e., Met-((Flp-Hyp-Gly)₇)-Met (SEQ ID NO:464);
SEQ ID NO:1 with a lysine residue attached at the N-terminus, i.e., Lys-((Pro-Pro-Gly)₇) (SEQ ID NO:465), at the C-terminus, i.e., (Pro-Pro-Gly)₇-Lys (SEQ ID NO:466), or at both termini, i.e., Lys-((Pro-Pro-Gly)₇)-Lys (SEQ ID NO:467);
SEQ ID NO:6 with a lysine residue attached at the N-terminus, i.e., Lys-((Flp-Hyp-Gly)₇) (SEQ ID NO:468), at the C-terminus, i.e., (Flp-Hyp-Gly)₇-Lys (SEQ ID NO:469), or at both termini, i.e., Lys-((Flp-Hyp-Gly)₇)-Lys (SEQ ID NO:470).

In other embodiments, the sequences set forth in SEQ ID Nos: 453-458 may have one or more methionine or lysine residues substituted in place of the one or more cysteine residues therein. In other embodiments, the sequences set forth in SEQ ID Nos: 459-464 may have one or more cysteine or lysine residues substituted in place of the one or more methionine residues therein. In other embodiments, the sequences set forth in SEQ ID Nos: 465-470 may have one or more methionine or cysteine residues substituted in place of the one or more lysine residues therein.

It will be understood by those of ordinary skill, of course, based on knowledge in the art and the teachings herein, that such CMPs may comprise two or more cysteine, methionine and/or lysine residues, in which at least one additional cysteine, methionine and/or lysine residue, or any combination thereof, may be substituted for at least one proline residue, at least one hydroxyproline residue, at least one fluoroproline residue and/or at least one chloroproline residue in any of the foregoing CMP sequences that comprise at least one proline, at least one hydroxyproline, at least one fluoroproline and/or at least one chloroproline residue. It also will be appreciated by those of ordinary skill in the art based on the teachings herein and information readily available in the art that other combinations of amino acid substitutions are also possible and within the scope of the present invention.

The CMPs described herein are suitable for a variety of purposes. For example, as described in further detail elsewhere herein, the CMPs may be used in a variety of therapeutic applications or preventative applications by being directly applied to or introduced into the body of a human or veterinary animal, particularly at sites of collagen disruption or potential collagen disruption, where the CMPs described herein will localize directly to the site of collagen disruption, anneal to disrupted collagen strands and stabilize the collagen structure such that it resists further disruption, and in some cases reform a native collagen triple helix in the site of collagen disruption. Such applications are useful in promoting the repair and strengthening of disrupted collagen in sites of injury or potential injury or disruption, for example in wounds, diseases, structural abnormalities or disorders (e.g., scarring, wrinkle formation, etc.) involving skin, tendon, ligament, cartilage, bone and other collagen-containing structures and organs. The CMPs described herein also are useful in providing biocompatible coatings for certain medical devices, to promote the healing of injuries and disorders in areas of the body where such devices are used in treating or preventing certain diseases, disorders and structural abnormalities or injuries in humans and veterinary animals, particularly those in which such diseases, disorders and structural abnormalities or injuries involve disruption of collagen and/or collagen-containing structures. The CMPs described herein also are useful in providing a unique delivery vehicle suitable for delivering a variety of therapeutic compounds, compositions and medicaments to sites of disease, disorder and structural abnormality or injury in humans and veterinary animals, particularly for use in treating, preventing or ameliorating diseases, disorders, medical conditions and structural abnormalities or injuries in which collagen disruption is either the cause of, is associated with, or is colocalized with the site of the disease, disorder and structural abnormality or injury. In additional embodiments, the CMPs described herein are useful in providing diagnostic agents suitable for diagnosing or detecting a disease, disorder, structural abnormality or injury in humans and veterinary animals. In certain such aspects, the CMPs may be either co-formulated with or conjugated directly or indirectly to one or more suitable diagnostic compounds, agents, labels and the like (see, e.g., U.S. Pat. Nos. 8,283,414, 8,883, 10,632,168, 11,389,513, 11,426,440 and 11,433,112, the disclosures of all of which are incorporated herein by reference in their entireties). Other suitable uses of the CMPs described herein and used in certain aspects of the present invention will be readily apparent to the ordinarily skilled artisan based on the disclosure herein and information that is readily available in the art.

In certain embodiments, the CMPs described herein are suitable for formation into a film, wafer, membrane or gel comprising one or more of the CMPs in a form suitable for introduction or implantation into a human or animal for therapeutic, preventative or diagnostic applications such as those described herein and others that will be familiar to those of ordinary skill in the relevant arts. For example, films, wafers, membranes, spheres, nanoparticles or gels can be formed from a solution of one or more of the CMPs described herein using methods such as those described in U.S. Pat. Nos. 6,197,934; 6,448,378; and 9,289,396; the disclosures of all of which are incorporated herein by reference in their entireties. Alternatively, films, wafers, membranes spheres, nanoparticles, or gels can be formed from other materials, such as atelocollagen (see U.S. Pat. Nos. 6,197,934; 6,448,378; and 9,289,396), copolymers of poly(lactic acid) and poly(glycoloic acid) (PLGA) (see Bala, I., et al., Crit. Rev. Ther. Drug Carrier Syst. 21(5):387-422 (2004)), and other materials that are known to those of ordinary skill in the art (see, e.g., Kumar, V., et al., eds., “Polymer Gels: Perspectives and Applications”, ISBN 978-981-10-6079-3, Singapore: Springer (2018)), and one or more of the CMPs can be suitably incorporated into such films, wafers, membranes, spheres, nanoparticles, gels, etc., during the formation thereof by including the CMPs in the solution, at concentrations of about 1%-99%, about 2%-95%, about 3%-90%, about 4%-90%, about 5%-90%, about 10%-90%, about 15%-90%, about 20%-90%, about 25%-90%, about 25%-85%, about 25%-75%, about 25%-50%, about 35%-50%, and the like. Suitable other amounts or concentrations of the CMPs described herein that can be suitably included in the solutions during formation of the films, wafers, membranes, spheres, nanoparticles, gels, etc., will be readily apparent from the teachings herein and from information readily available in the art to the ordinarily skilled artisan. In certain such embodiments, one or more therapeutic compounds described herein, and/or one or more CMP-TC conjugates described herein, can be suitably incorporated into the solution from which the films, wafers, membranes, spheres, nanoparticles, gels, etc., are formed. Alternatively, in related aspects, one or more films, wafers, membranes, spheres, nanoparticles, gels, etc., once formed as described above, can be treated or coated with one or more CMPs and/or CMP-TC conjugates described herein, by immersing the films, wafers, membranes, spheres, nanoparticles, gels, etc., in a solution, particularly a buffered aqueous solution, containing a suitable amount or concentration (such as those described herein) of one or more CMPs or CMP-TC conjugates described herein, and then drying the films, wafers, membranes, etc., prior to use in therapeutic, preventative or diagnostic methods such as those described herein.

### Attachment/Conjugation of CMPs

In certain embodiments of the invention, the CMPs described herein are suitably attached or conjugated to one or more therapeutic or diagnostic compounds, to produce CMP conjugate compounds. In such embodiments of the invention, the CMP-therapeutic compound or CMP-diagnostic compound conjugate compounds can then be introduced into the body of a human or veterinary animal, in methods of treating and/or preventing and/or diagnosing certain diseases, disorders and structural abnormalities in humans or veterinary animals suffering therefrom. Accordingly, in certain embodiments the present invention also provides the use of the CMPs described herein attached or conjugated to one or more therapeutic compounds to produce conjugated CMPs, compositions comprising such conjugated CMPs (which may optionally comprise one or more additional therapeutic or pharmaceutically active ingredients), methods of producing such conjugates and methods of using such conjugates and compositions in the treatment, prevention and diagnosis of a variety of diseases, disorders and medical conditions in humans and veterinary animals.

Conjugates of CMPs and at least one therapeutic compound (which may be described herein as “CMP-TC conjugates”) according to this aspect of the invention will comprise at least one CMP described herein attached to at least one therapeutic compound to form a CMP-TC conjugate. CMPs suitably used in such aspects of the invention include any of those described herein, including CMPs having an amino acid sequence corresponding to any one of SEQ ID NOs:1-387 and particularly wherein the CMPs have an amino acid sequence corresponding to any one of SEQ ID NOs:1-14, 66-94, 107-135, 136-140, 192-220, 233-261, 260-264, 280, 281, 293, 294, 306, 307, 318-346, 347, 348, 359-388, and 397-416, and more particularly CMPs having amino acid sequences corresponding to SEQ ID NOs:10-27, 81-94, 122-135, 207-220, 248-261, 333-346, 374-388 and 397-416. Other suitable CMP sequences will be immediately apparent to one of ordinary skill in the art based on the teachings contained herein. For example, a CMP having at least one, and in some cases more than one, cysteine, methionine or lysine residue substituted in place of at least one, and in some cases more than one, proline, hydroxyproline, fluoroproline or chloroproline residue in SEQ ID NOs:1-9, will be particularly suitable for use in producing the CMP-TC conjugates provided by and used in the present invention. Examples of such suitable CMPs include those having amino acid sequences corresponding to SEQ ID NOs: 10-27, 81-94, 122-135, 207-220, 248-261, 333-346, 374-388, 397-416 and 453-470.

In other such embodiments, the CMPs may be attached to one or more other peptides or proteins via one or more peptide bonds at the C- and/or N-terminus of the CMP, producing a CMP-TC conjugate that is a peptide or protein comprising the CMP. Any peptide or protein, regardless of whether such peptide or protein is in itself a therapeutic peptide or protein, may be advantageously attached or conjugated via one or more peptide bonds to the CMP, resulting in a new compound that is a combination of the CMP and the peptide or protein and that may have a bioactivity that arises from the combination of the structures and functions of the CMP and the protein or peptide to which it has been conjugated. For example, a CMP that has been conjugated to an Fc or Fab fraction of an antibody (e.g., a polyclonal antibody or monoclonal antibody) may exhibit the bioactivity of both the CMP and of the antibody fraction, thereby providing a unique compound that may have multiple functionalities which may enhance the therapeutic and/or diagnostic utility of the compound above and beyond that of the CMP or antibody fragment alone, or may permit the directed delivery of the therapeutic or diagnostic to a particular cell, tissue or organ type when introduced into the body of an animal, particularly a human or veterinary animal, for use in a variety of therapeutic and/or diagnostic methods such as those described elsewhere herein. Other peptides or proteins that themselves have therapeutic and/or diagnostic utility may also be advantageously attached or conjugated to the CMPs according to this aspect of the invention. Such peptides or proteins include those in certain pharmacological classes including but not limited to antithrombins, fibrinolytic peptides/proteins, enzymes, antineoplastic agents, hormones, fertility agents, immunosuppressive agents (including anti-inflammatory agents), bone-related peptides/proteins, antidiabetic agents and antibodies. More specific examples of peptides/proteins falling within these classes include those that have been approved by the U.S. Food & Drug Administration as therapeutics and/or diagnostics, such as those listed in the real-time THPdb database of FDA approved therapeutic peptides and proteins available at https://webs.iiitd.edu.in/raghava/thpdb/category.php, and as first published in Usmani, S. S. et al., PLoS ONE 12(7): e0181748 (2017), https://doi.org/10.1371/journal.pone.0181748, which database and publication are incorporated herein by reference in their entireties. Other suitable peptides and proteins, and fragments thereof, that may be advantageously conjugated or attached to CMPs as described herein will be familiar to the ordinarily skilled artisan in view of the teachings herein and of information readily available in the relevant arts.

Methods of preparing the CMPs and CMP-TCs described herein and provided and used in the present invention will be familiar to those of ordinary skill in the art based on the teachings herein and information that is readily available in the art. For example, CMPs can be synthesized using standard protein/peptide synthesis techniques such as those described in U.S. Pat. Nos. 5,973,112; 7,122,521; and 7,858,741; as well as in U.S. Patent Publ. No. US 2007/0275897 A1, the disclosures of all of which are incorporated herein by reference in their entireties. Synthesis of CMPs can also be accomplished by purchasing custom-synthesized CMPs produced commercially, for example by Bachem (Torrance, CA, USA) and RS Synthesis (Louisville, KY, USA). In other embodiments, synthesis of CMPs can be accomplished using genetic engineering and recombinant expression of the CMPs from prokaryotic or eukaryotic expression systems (see, e.g., Buechter, D. D., et al., J. Biol. Chem. 278(1):645-650 (2003)).

In synthesizing the peptides described herein, in certain embodiments it is preferred that certain stereochemistries be used for the amino acid substitutions, particularly if hydroxyproline, fluoroproline or chloroproline are used:
(1) if hydroxyproline is substituted in place of proline in the Xaa position of the Xaa-Yaa-Gly trimer noted hereinabove, in certain embodiments the hydroxyproline has a (2R, 4S) stereochemistry, or a cis or trans, and preferably a cis, stereochemistry;
(2) if hydroxyproline is substituted in place of proline in the Yaa position of the Xaa-Yaa-Gly trimer noted hereinabove, in certain embodiments the hydroxyproline has a (2R, 4S) stereochemistry, or a cis or trans, and preferably a cis, stereochemistry;
(3) if fluoroproline is substituted in place of proline in the Yaa position of the Xaa-Yaa-Gly trimer noted hereinabove, in certain embodiments the hydroxyproline has a (2R, 4S) stereochemistry, or a cis or trans, and preferably a cis, stereochemistry; and
(4) if chloroproline is substituted in place of proline in the Yaa position of the Xaa-Yaa-Gly trimer noted hereinabove, in certain embodiments the hydroxyproline has a (2R, 4S) stereochemistry, or a cis or trans, and preferably a cis, stereochemistry.

Other suitable stereochemistries can be determined empirically without having to resort to undue experimentation, and will be immediately apparent to those of ordinary skill in the art. As noted above, certain CMPs provided by and used in the present invention may contain one or more additional substitutions, for example one or more cysteine residues and/or one or more methionine residues, in place of one or more prolines in a given CMP multimer. Such substitutions are suitably accomplished by adding those residues to the growing CMP peptide chain during the synthetic process using standard peptide synthetic methods such as those described elsewhere herein and those that are known in the art.

Once the CMPs have been prepared, they are suitably used in producing the CMP-TCs of the invention, i.e., the therapeutic or diagnostic compositions of the invention, by attaching one or more therapeutic compounds to the CMPs. In certain embodiments, the CMP-TCs of the invention can be prepared a method comprising (a) providing a collagen mimetic peptide having an amino acid sequence corresponding to any one of SEQ ID NOs:1-470, particularly CMPs have an amino acid sequence corresponding to any one of SEQ ID NOs:1-14, 66-94, 107-135, 136-140, 192-220, 233-261, 260-264, 280, 281, 293, 294, 306, 307, 318-346, 347, 348, 359-388, 397-416 and 453-470, and more particularly CMPs having amino acid sequences corresponding to SEQ ID NOs:10-27, 81-94, 122-135, 207-220, 248-261, 333-346, 374-388, 397-416 and 453-470; (b) providing at least one therapeutic or diagnostic compound suitable to be conjugated to the CMP; and (c) attaching the therapeutic or diagnostic compound directly or indirectly to the CMP. In certain cases, particularly wherein the therapeutic compound is a small peptide biologic compound, the therapeutic compound can be directly attached to the CMP via a peptide bond, for example by simply extending the synthesis of the peptide beyond the carboxy terminus of the CMP and attaching the amino terminal amino acid of the therapeutic compound to the carboxy terminal amino acid of the CMP via a peptide bond. One example of such a CMP-TC is a peptide conjugate in which the wound healing peptide known as Substance P and having an amino acid sequence of Arg-Pro-Lys-Pro-Gln-Gln-Phe-Phe-Gly-Leu-Met (SEQ ID NO:471), is attached to a CMP described herein. Examples of such conjugates include, for example:

(SEQ ID NO: 472)
Pro-Pro-Gly-Pro-Pro-Gly-Pro-Pro-Gly-Pro-Pro-Gly-
Pro-Pro-Gly-Pro-Pro-Gly-Pro-Pro-Gly-Arg-Pro-Lys-
Pro-Gln-Gln-Phe-Phe-Gly-Leu-Met;
(SEQ ID NO: 473)
Hyp-Pro-Gly-Hyp-Pro-Gly-Hyp-Pro-Gly-Hyp-Pro-Gly-
Hyp-Pro-Gly-Hyp-Pro-Gly-Hyp-Pro-Gly-Arg-Pro-Lys-
Pro-Gln-Gln-Phe-Phe-Gly-Leu-Met;
(SEQ ID NO: 474)
Pro-Hyp-Gly-Pro-Hyp-Gly-Pro-Hyp-Gly-Pro-Hyp-Gly-
Pro-Hyp-Gly-Pro-Hyp-Gly-Pro-Hyp-Gly-Arg-Pro-Lys-
Pro-Gln-Gln-Phe-Phe-Gly-Leu-Met;
(SEQ ID NO: 475)
Flp-Hyp-Gly-Flp-Hyp-Gly-Flp-Hyp-Gly-Flp-Hyp-Gly-
Flp-Hyp-Gly-Flp-Hyp-Gly-Flp-Hyp-Gly-Arg-Pro-Lys-
Pro-Gln-Gln-Phe-Phe-Gly-Leu-Met;
(SEQ ID NO: 476)
Clp-Hyp-Gly-Clp-Hyp-Gly-Clp-Hyp-Gly-Clp-Hyp-Gly-
Clp-Hyp-Gly-Clp-Hyp-Gly-Clp-Hyp-Gly-Arg-Pro-Lys-
Pro-Gln-Gln-Phe-Phe-Gly-Leu-Met;
(SEQ ID NO: 477)
Pro-Flp-Gly-Pro-Flp-Gly-Pro-Flp-Gly-Pro-Flp-Gly-
Pro-Flp-Gly-Pro-Flp-Gly-Pro-Flp-Gly-Arg-Pro-Lys-
Pro-Gln-Gln-Phe-Phe-Gly-Leu-Met;
and
(SEQ ID NO: 478)
Pro-Clp-Gly-Pro-Clp-Gly-Pro-Clp-Gly-Pro-Clp-Gly-
Pro-Clp-Gly-Pro-Clp-Gly-Pro-Clp-Gly-Arg-Pro-Lys-
Pro-Gln-Gln-Phe-Phe-Gly-Leu-Met.

In other methods of the invention, the one or more therapeutic or diagnostic compounds are suitably conjugated or attached to the CMPs via a covalent bond other than a peptide bond (see, e.g., U.S. Pat. Nos. 3,283,414 and 3,883,964, which are incorporated herein by reference in their entireties). For example, therapeutic compounds can be attached directly to a cysteine or methionine residue on a CMP described herein by covalently bonding a hydroxyl or amino group on an amino acid residue (e.g., a lysine residue) on the therapeutic or diagnostic compound (if it is a biologic molecule) to a sulfhydryl group on the cysteine or methionine residue of the CMP. Alternatively, if the CMP does not contain a cysteine or methionine residue, the one or more therapeutic or diagnostic compounds can be attached or conjugated to the CMP by a reaction between a hydroxyl group or amino group on the CMP and a sulfhydryl group on an amino acid residue (e.g., at a cysteine or methionine residue) on the therapeutic or diagnostic compound (if it is a biologic molecule). In yet another alternative method of conjugation, therapeutic compounds can be attached directly to a lysine residue on a CMP described herein by covalently bonding the therapeutic compound to an amino group on the lysine, for example using NHS ester conjugation (see, e.g., Mattson, G., et al., Molec. Biol. Rep. 17:167-183 (1993); Grabarek, Z. and Gergely, J., Anal. Biochem. 185:131-135 (1990); Staros, J. V. et al., Anal. Biochem. 156:220-2 (1986); Timkovich, R., Anal. Biochem. 79:135-43 (1977)). Such direct covalent attachments or conjugations between the CMP and the therapeutic/diagnostic compound can be accomplished using standard reaction techniques that will be familiar to those of ordinary skill in organic chemistry.

In other embodiments, particularly those wherein the therapeutic or diagnostic compound is not a biologic (and therefore does not have a peptide structure or amino acid residues having groups suitably attachable to cysteine, methionine, lysine or other residues on the CMP), such as small molecule organic or inorganic therapeutic or diagnostic compounds, the at least one therapeutic or diagnostic compound is indirectly attached to the collagen mimetic peptide via use of an attachment means. In such embodiments, the attachment means has two attachable ends, one of which attaches to an amino acid residue, and suitably a sulfhydryl group on a cysteine or methionine residue or an amino group on a lysine residue, of a CMP, and the other of which attaches to a hydroxyl or amino group on the therapeutic or diagnostic compound. For example, in certain such embodiments the attachment means comprises at least one polymeric chain having a first end and a second end, and the first end of the polymeric chain binds to the sulfhydryl group on a cysteine or methionine residue or an amino group on a lysine residue on the collagen mimetic peptide and the opposite or second end of the polymeric chain binds to an amino group or hydroxyl group on the therapeutic compound. In embodiments where the therapeutic or diagnostic compound is a biologic that is not suitable for direct attachment via peptide synthesis as described elsewhere herein, the second end of the attachment means can be attached to an amino group on an amino acid residue, such as a lysine residue, on the biologic therapeutic or diagnostic compound. Suitable such attachment means are well-known to those of ordinary skill in the art. For example, one attachment means suitable for use in accordance with this aspect of the invention includes a moiety which is a polymeric chain that on one end (the CMP-binding end in particular) comprises a sulfhydryl-binding group such as a maleimide, and on the other end (the therapeutic or diagnostic compounding-binding end in particular) comprises an amino-binding group such as N-hydroxysuccinimide. In certain such embodiments, the polymeric chain is a linear polyethyleneglycol chain comprising at least four ethyleneglycol monomers, e.g., from four to fifty ethyleneglycol monomers, from ten to forty ethyleneglycol monomers, from fifteen to thirty ethyleneglycol monomers, from fifteen to twenty-five ethyleneglycol monomers, from twenty to twenty-five ethyleneglycol monomers, and particularly four, six, eight, twelve, twenty, twenty-two, twenty-three, twenty-four or twenty-five ethyleneglycol monomers. Such attachment means suitable for attaching one or more therapeutic or diagnostic compounds to a CMP by the methods described herein are available commercially, e.g., from Thermo Fisher Scientific (Waltham, MA) (e.g., SM(PEG)6, SM(PEG)8, SM(PEG)12 and SM(PEG)24). By adjusting the length of the polymer chain, the bioavailability and sustainability of the therapeutic or diagnostic compound in vivo can be modulated—the use of longer polymer chains, e.g., a polymer comprising 24 ethyleneglycol monomers, will increase the rate of bioavailability of the compound once the CMP-TC has been introduced into the body of the human or veterinary animal, while the use of shorter polymer chains, e.g., a polymer comprising six ethyleneglycol monomers, will decrease the rate of bioavailability and thus increase the sustainability (or, in other words, will result in delayed release or sustained release) of the therapeutic or diagnostic compound. Other conjugates using linear or star-shaped PEG moieties which may be suitably prepared using the CMPs of the present invention, and used in the therapeutic and diagnostic methods of the invention, are disclosed in U.S. Pat. Nos. 8,283,414 and 8,883,964, which are incorporated herein by reference in their entireties. Hence, according to certain such aspects of the invention, the at least one therapeutic compound comprises at least one reactive hydroxyl group capable of being cross-linked to the collagen mimetic peptide using a polymeric linker.

Other indirect attachment methods for conjugating the one or more therapeutic or diagnostic compounds into or onto the CMPs also are suitably used according to the invention. For example, the at least one therapeutic or diagnostic compound can be enclosed within at least one nanoparticle that is attached via an attachment means, such as those described herein, to the collagen mimetic peptide. Alternatively, the collagen mimetic peptide can suitably comprise at least one biotin moiety and the therapeutic molecule can suitably comprise at least one avidin or streptavidin moiety, and the biotin moiety on the collagen mimetic peptide will bind to the avidin or streptavidin moiety on the therapeutic or diagnostic compound, thereby attaching the collagen mimetic peptide to the therapeutic or diagnostic compound. Of course, the alternative is also suitable for use, in which the collagen mimetic peptide can suitably comprise at least one avidin or streptavidin moiety and the therapeutic or diagnostic compound can suitably comprise at least one biotin moiety, and the biotin moiety on the at least one therapeutic or diagnostic compound will bind to the avidin or streptavidin moiety on the collagen mimetic peptide, thereby attaching the collagen mimetic peptide to the therapeutic compound.

Thus, according to certain embodiments of the invention, the therapeutic or diagnostic compounds can be suitably attached directly to the CMPs described herein. In other embodiments of the invention, the one or more therapeutic or diagnostic compounds can be attached indirectly to the CMPs described herein, for example via the use of a spacer, linker or bridge moiety. It is to be understood that whether the one or more therapeutic compounds are attached directly or indirectly to the CMPs, such attachment results in the production of conjugates of the CMPs and the one or more therapeutic compounds, which may be defined herein as CMP-TC conjugates.

Suitable therapeutic or diagnostic compounds for attachment or conjugation to the CMPs to produce the CMP-TCs of the present invention include any compound that has been shown to have particular therapeutic or preventative properties against one or more diseases, disorders, physical conditions or afflictions when introduced into a human or veterinary animal suffering from or predisposed to such diseases, disorders, physical conditions or afflictions. Provided that the therapeutic or diagnostic compound is capable of being conjugated or attached to at least one CMP according to the teachings herein, any therapeutic or diagnostic compound can be used in the conjugates, compositions and methods of the present invention. Suitable such therapeutic compounds may be biologic or non-biologic (e.g., so-called “small molecule”) therapeutic compounds. Compounds suitable for use include, but are not limited to, a steroidal anti-inflammatory drug, (e.g., prednisolone or a pharmaceutically acceptable salt thereof, such as prednisolone acetate), a nonsteroidal anti-inflammatory drug (e.g., acetylsalicylic acid, acetaminophen, ibuprofen, naproxen, nepafenac, bromfenac, diclofenac, flurbiprofen, ketoprofen, ketorolac, and an indene derivative (e.g., indomethacin, sulindac (Clinoril) and the like; see, e.g., U.S. Pat. No. 7,601,874, which is incorporated herein by reference in its entirety, for other indene derivatives suitably used as active pharmaceutical ingredients), and pharmaceutically acceptable salts, esters and derivatives thereof), a topical anesthetic (e.g., tetracaine, lidocaine, oxybuprocaine, proparacaine, and the like), a vitamin or a vitamin derivative or vitamin precursor (e.g., retinol, tretinoin, retinal, carotene and other retinoids and retinoid derivatives or precursors; folate; α-tocopherol; calciferol; phylloquinone, menadione and other vitamin K forms, precursors or derivatives, ascorbate; and the like), a therapeutic enzyme or a therapeutic fragment thereof (e.g., a collagenase and a serine protease, or a therapeutically effective fragment thereof), an antibiotic (e.g., an aminoglycoside antibiotic (such as gentamycin, tobramycin, paromomycin, kanamycin, neomycin and amikacin, and a pharmaceutically acceptable salt or ester thereof, e.g., tobramycin sulfate), a fluoroquinolone antibiotic (such as moxifloxacin, gatifloxacin, levofloxacin, gemifloxacin, ciprofloxacin, norfloxacin and ofloxacin, and a pharmaceutically acceptable salt, ester or derivative thereof, e.g., moxifloxacin hydrochloride, ciprofloxacin hydrochloride and gatifloxacin hydrochloride), a sulfonamide antibiotic (such as sulfacetamide, sulfadiazine, sulfadimidine, sulfafurazole (sulfisoxazole), sulfisomidine (sulfaisodimidine), sulfadoxine, sulfamethoxazole, sulfamoxole, sulfanitran, sulfadimethoxine, sulfamethoxypyridazine, sulfametoxydiazine, sulfametopyrazine and terephtyl, and a pharmaceutically acceptable salt, ester or derivative thereof), a β-lactam antibiotic (such as a penicillin or a derivative thereof (for example penicillin G, penicillin V, a benzylpenicillin and phenoxymethylpenicillin), dicloxacillin, flucloxacillin, oxacillin, nafcillin, amoxicillin, an ampicillin, ticarcillin, piperacillin, ritipenem, a carbapenem (e.g., ertapenem, doripenem, imipenem and meropenem, and a pharmaceutically acceptable salt, ester or derivative thereof), a cephem (such as cefazolin, cefalexin, cefadroxil, cefapirin, cefaclor, cefotetan, cephamycin (cefoxitin), cefprozil, cefuroxime axetil, ceftriaxone, ceftazidime, cefoperazone, cefdinir, cefcapene, cefdaloxime, ceftizoxime, cefmenoxime, cefotaxime, cefpiramide, cefpodoxime, ceftibuten, cefditoren, cefepime, ceftaroline fosamil, ceftolozane, ceftobiprole, ceftiofur, cefquinome and cefovecin, and a pharmaceutically acceptable salt, ester or derivative thereof), a monobactam (such as aztreonam or a pharmaceutically acceptable salt, ester or derivative thereof) and a β-lactamase inhibitor (such as sulbactam, tazobactam, clavulanic acid and avibactam, and a pharmaceutically acceptable sat, ester or derivative thereof)) or a cyclic peptide antibiotic (such as cyclosporine), a therapeutic monoclonal antibody or a therapeutic fragment thereof (such as adalimumab, altumomab, atezolizumab, atlizumab, bevacizumab, canakinumab, catumaxomab, certolizumab, cetuximab, clivatuzumab, edrecolomab, efalizumab, fontolizumab, girentuximab, golimumab, infliximab, labetuzumab, MABpl (Xilonix™) natalizumab, nimotuzumab, nivolumab, oregovomab, panitumumab, pembrolizumab, pemtumomab, pertuzumab, ramucirumab, ranibizumab, rituximab, ruplizumab, tracatuzumab, tocilizumab, trastuzumab, ustekinumab, vedolizumab, visilizumab, votumumab, zalutumumab and zanolimumab, and active fragments, combinations or conjugates thereof), a therapeutic fusion protein (in certain embodiments, a recombinant fusion protein such as aflibercept (Regeneron), etanercept (Amgen), alefacept (Astellas Pharma), abatacept (Bristol-Myers Squibb), rilonacept (Regeneron), romiplostim (Amgen) and belatacept (Bristol-Myers Squibb)), a prostaglandin analogue (such as latanoprost, travoprost, tafluprost, unoprostone, netarsudil, tatanoprostene bunod, netarsudil and bimatoprost, and pharmaceutically acceptable salts, esters and derivatives thereof), a growth factor (such as EGF, PDGF, TGF-β, IGF-1, VEGF, FGF-β, IGF-1) or a therapeutic or growth-promoting (particularly skin growth-promoting) fragment thereof, a neuropeptide (such as Substance P (SEQ ID NO:389), an α-adrenergic antagonist (such as brimonidine, clonidine and apraclonidine, and pharmaceutically acceptable salts, esters or derivatives thereof), a β-adrenergic antagonist (such as timolol, propranolol, atenolol, levobunolol, carteolol, betaxolol, and pharmaceutically acceptable salts, esters and derivatives thereof, e.g., timolol maleate), a cell surface receptor antagonist (such as lifitegrast or etanercept), a carbonic anhydrase inhibitor (such as dorzolamide, brinzolamide, methazolamide and acetazolamide, and pharmaceutically acceptable salts, esters and derivatives thereof, e.g., dorzolamide hydrochloride), and pharmaceutically acceptable salts, esters and derivatives thereof. With certain such therapeutic compounds, administration simultaneously with the CMPs described herein, whether as a CMP-TC conjugate or simply with one or more CMPs and one or more TCs in an admixture or applied separately, may prevent, attenuate or lessen one or more adverse side effects of the therapeutic compound. For example, it is known that the therapeutic administration of certain fluoroquinolone antibiotics may cause damage to collagen and collagen-containing structures (e.g., tendons) in humans or veterinary animals who have been treated with fluoroquinolones (see, e.g., “FDA Drug Safety Communication: FDA updates warnings for oral and injectable fluoroquinolone antibiotics due to disabling side effects,” accessed Nov. 6, 2017, at https://www.fda.gov/Drugs/DrugSafety/ucm511530.htm). As a result, simultaneous or co-administration of one or more of the CMPs described herein with one or more fluoroquinolone antibiotics to a human or veterinary animal in need of treatment with fluoroquinolones may allow the patient to receive the therapeutic benefits of the fluoroquinolone while mitigating, ameliorating or avoiding the collagen disruption resulting from such therapy, as the CMP can localize to and repair areas of damaged collagen in vivo.

Other suitable therapeutic compounds for use in the CMP-TC compounds, compositions and conjugates of the present invention include other non-biologic small molecule therapeutic compounds, including but not limited to alkylating agents, anti-tumor antibiotics, antimetabolites, hormonal agents, plant alkaloids, angiogenesis inhibitor, GnRH agonists, tyrosine kinase inhibitors, and the like. Examples of such non-biologic small molecule therapeutic compounds suitably used in accordance with the invention include but are not limited to a nitrosourea, a lenalidomide, imatinib, penatrexed, bortexomib, abiraterone acetate, everolimus, taxol, docetaxel, paclitaxel, carbazitaxel, mitoxantrone, carboplatin, cisplatin, gemcitabine, doxorubicin, casodex, flutamide, enzalutamide, abiraterone, sipuleucel-T and ketoconazole. Other suitable non-biologic small molecule therapeutic compounds that are advantageously used in forming the CMP-TC conjugates of the present invention, particularly for producing CMP-TC conjugates that are useful in treating certain cancers and preventing tumor metastasis, include inhibitors of lysyl oxidase (LOX), lysyl oxidase-like 1 (LOXL1) and lysyl oxidase-like 2 (LOXL2) enzymes. Such inhibitors have been suggested to have potential therapeutic application in treating and/or preventing certain cancers and the metastasis of solid tumors (see, e.g., U.S. Pat. Nos. 5,201,456; 5,120,764; 5,252,608; 8,461,303; 8,658,167; 8,680,246; 9,176,139; 9,255,086; and 9,289,447; see also Erler, J. T., et al., Nature 440:1222-1226 (2006); Erler, J. T., et al., Cancer Cell 15(1):35-44 (2009); Bondareva, A., et al., PLoS ONE 4(5):e5620 (2009); Granchi, C., et al., ChemMedChem 4(10:1590-1594 (2009); and Fang, M., et al., Tumor Biol. 35:2871-2882 (2014); the disclosures of all of which are incorporated herein by reference in their entireties). In related aspects of the invention, CMP-TC conjugates comprising one or more inhibitors of LOX or LOX-like enzymes are suitably used in treating and/or preventing certain fibrotic diseases and disorders that are mediated by oxidoreductase enzymes such as LOX and the LOX-like enzymes (e.g., LOXL1 and LOXL2) in humans and veterinary animals. Fibrotic diseases and disorders suitably treated and/or prevented according to this aspect of the invention include but are not limited to pulmonary fibrosis, liver cirrhosis, myocardial fibrosis, surgical scarring, systemic sclerosis, scleroderma, keloid formation, proliferative vitreo retinopathy, and other fibrotic diseases and disorders that will be familiar to those of ordinary skill in the relevant arts. Particularly useful inhibitors of LOX and the Lox-like proteins include β-aminopropionitrile and certain derivatives and prodrugs thereof (see, e.g., U.S. Pat. Nos. 5,201,456; 5,120,764; 5,252,608; 8,461,303; 8,680,246; 9,176,139; and 9,255,086; the disclosures of all of which are incorporated herein in their entireties), as well as antibodies (which may be polyclonal or, preferably monoclonal) and fragments or portions thereof which bind to and inhibit the activity or function of LOX and LOX-like enzymes (see, e.g., U.S. Pat. No. 8,461,303; the disclosure of which is incorporated herein in its entirety).

In additional embodiments, compounds or compositions can be prepared comprising one or more CMPs and one or more antigens, either in admixture or co-formulation of one or more CMPs with one or more antigens (and optionally with one or more pharmaceutically suitable carriers or excipients), or in other compounds or compositions in which the one or more antigens are linked or conjugated directly or indirectly to the one or more CMPs. According to certain such aspects, the antigen may be a complete antigen or antigenic determinant or a fragment thereof (e.g., a hapten) that is capable of inducing an immune response in a human or veterinary animal when presented in the appropriate physiological context to the immune system of the human or veterinary animal, such as in the form of administration of the compound, conjugate or composition in the form of a vaccine or immunization to the human or veterinary animal. Compounds, conjugates and compositions useful in such embodiments can be prepared via co-formulation or direct or indirect conjugation according to the methods described elsewhere herein for co-formulation and conjugation of therapeutic compounds with or to CMPs. Antigens or portions thereof suitable for use in such compounds, conjugates and compositions, and therefore in methods of use thereof, include any molecule or particle, or portion thereof, that is capable of inducing an immune response in the human or veterinary animal, including but not limited to antigens (e.g., proteins, toxins, lipids, and other antigenic moieties, molecules or complexes) arising from or produced by bacteria (in which the antigen may comprise the entire bacterium or a portion thereof, such as a cell wall or cell membrane component, a nuclear component or a toxin produced by the bacterium), viruses (in which the antigen may comprise the entire viral particle or a portion thereof, such as a coat component (e.g., a protein or lipid or portion thereof), a nuclear component, or an enzyme encoded by or which is a part of the viral particle), protists, fungi, plants (which may include plant irritants or allergens such as pollen particles), animals (from which the antigen or portion thereof may be an allogeneic antigen or autogeneic antigen, or a portion thereof), and the like; examples of such antigens or portions thereof will be readily familiar to those in the relevant arts. Such compounds, compositions or conjugates are suitably used in methods for treating and/or preventing one or more disorders, diseases and afflictions in humans and veterinary animals, for example through the use of the compounds, compositions or conjugates in creating an immune response in the animal or veterinary human. In certain such methods, a disease or disorder is treated and/or prevented in the animal or veterinary animal by administration of one or more of the compounds, compositions or conjugates of this aspect of the invention into the human or veterinary animal, such as in the form of a vaccine or immunization. Such vaccines or immunizations are suitably formulated according to methods that are well-known in the relevant arts, and are administered in any mode that will result in the development of an immune response by the human or veterinary animal to the antigen or portion thereof, thereby treating and/or preventing the disease or disorder caused directly or indirectly by the antigen or portion thereof. Such vaccines or immunizations can be administered to the human or veterinary animal by any suitable route, such as orally, parenterally (including subcutaneously, intradermally, transdermally, intrathecally or intravenously), via ocular administration (e.g., in the form of drops, gels, wafers, or via injection, as described elsewhere herein for CMP-TC administration to the eye), intranasally, and other routes of administration that will be familiar to those of ordinary skill. In such embodiments, the compounds, conjugates or compositions of the invention are suitably administered to the human or veterinary animal until an immune response is developed by the human or veterinary animal that is sufficient to treat and/or prevent the target disease or disorder, and may be readministered as necessary to boost the immune response and/or to ensure continued immunity to the target antigen or portion thereof.

Diseases and disorders suitably treated by such methods of the invention include any disease or disorder involving or resulting from the activity of any foreign agent acting upon the cells, organs, organ systems, bodily structures or bodies of humans and veterinary animals, including but not limited to infectious diseases, cancers, allergies and other immune overreactions (e.g., graft-versus-host or host-versus-graft diseases), Stevens-Johnson Syndrome, mucus membrane pemphigoid, toxic epidermal necrolysis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, and the like.

Suitable diagnostic compounds for attachment or conjugation to CMPs to produce the conjugates and compositions of the invention include, but are not limited to, labeled probes, such as fluorescent dyes (e.g., quantum dots, indocyanine green, fluorescein, rhodamine, a merocyanine dye, a near-infrared fluorescent dye, and the like); a radioisotope, a nuclide used for PET, a nuclide used for SPECT, particularly wherein each of the radioisotope, the nuclide used for PET or SPECT is selected from the group consisting of ¹¹C, ¹³N, ¹⁵O, ¹⁸F, ⁶⁶Ga, ⁶⁷Ga, ⁶⁸Ga, ⁶⁰Cu, ⁶¹Cu, ⁶²Cu, ⁶⁷Cu, ⁶⁴Cu, ⁴⁸V, Tc-99m, ²⁴¹Am, ⁵⁵Co, ⁵⁷Co, ¹⁵³Gd, ¹¹¹In, ¹³³Ba, ⁸²Rb, ¹³⁹Ce, Te-123m, ¹³⁷Cs, ⁸⁶Y, ⁹⁰Y, ^{185/187}Re, ^{186/188}Re, ¹²⁵I, a complex thereof, and a combination thereof, and an MRI contrast medium, a CT contrast medium, and a magnetic material, particularly wherein each of the MRI contrast medium, the CT contrast medium, and the magnetic material is selected from the group consisting of gadolinium, Gd-DTPA, Gd-DTPA-BMA, Gd-HP-D03A, iodine, iron, iron oxide, chromium, manganese, a complex or chelate complex thereof, and a combination thereof. According to such aspects of the invention, the CMP and the labeled probe are suitably physically or chemically bound directly to each other, for example via a direct conjugation through a coordinate bond, a covalent bond, a hydrogen bond, a hydrophobic interaction or a physical adsorption, or indirectly via use of at least one attachment means such as those described herein and others that are known in the art. Methods of conjugating or attaching diagnostic compounds to proteins, such as CMPs, are known in the art (see, e.g., U.S. Publ. Patent Appl. No. US 2012/0195828 A1, the disclosure of which is incorporated herein in its entirety).

### Use of CMPs and CMP-TC Conjugates

Thus, the invention provides methods of preparing compositions that are useful in treating, preventing, diagnosing or ameliorating a disease, disorder or medical condition in humans or veterinary animals. In yet another aspect, the invention provides methods of treating, preventing, diagnosing or ameliorating a disease, disorder or medical or physical condition in humans or veterinary animals using the compositions of the invention. Particularly preferred CMPs for use in such aspects of the invention include CMPs comprising, consisting essentially of, or consisting of, CMPs having an amino acid sequence of (Pro-Pro-Gly)₇ (SEQ ID NO:1), (Flp-Pro-Gly)₇ (SEQ ID NO:4), (Pro-Flp-Gly)₇ (SEQ ID NO:5), (Flp-Hyp-Gly)₇ (SEQ ID NO:6), (Clp-Hyp-Gly)₇ (SEQ ID NO:9), (Hyp-Flp-Gly)₇ (SEQ ID NO:388), Gly₃-(Pro-Hyp-Gly)₆ (SEQ ID NO:397), Gly₃-(Pro-Flp-Gly)₆ (SEQ ID NO:398), Gly₃-(Pro-Hyp-Gly)₇ (SEQ ID NO:399), Gly₃-(Pro-Flp-Gly)₇ (SEQ ID NO:400), Gly₃-(Pro-Hyp-Gly)₈ (SEQ ID NO:401), Gly₃-(Pro-Flp-Gly)₅ (SEQ ID NO:402), Gly₃-(Pro-Hyp-Gly)₉ (SEQ ID NO:403), Gly₃-(Pro-Flp-Gly)₉ (SEQ ID NO:404), (Pro-Hyp-Gly)₆-Tyr (SEQ ID NO:405), (Pro-Flp-Gly)₆-Tyr (SEQ ID NO:406), (Pro-Hyp-Gly)₇-Tyr (SEQ ID NO:407), (Pro-Flp-Gly)₇-Tyr (SEQ ID NO:408), (Pro-Hyp-Gly)₈-Tyr (SEQ ID NO:409), (Pro-Flp-Gly)₈-Tyr (SEQ ID NO:410), Cys-(Pro-Hyp-Gly)₃ (SEQ ID NO:411), Cys-(Pro-Flp-Gly)₃ (SEQ ID NO:412), Cys-(Pro-Hyp-Gly)₅ (SEQ ID NO:413), Cys-(Pro-Flp-Gly)₅ (SEQ ID NO:414), Cys-(Pro-Hyp-Gly)₇ (SEQ ID NO:415), or Cys-(Pro-Flp-Gly)₇ (SEQ ID NO:416), and derivatives thereof comprising one or more cysteine, methionine or lysine residues such as those described elsewhere herein.

The CMPs and CMP-TC conjugates of the present invention, including solutions, gels, films, wafers, membranes, spheres, nanoparticles and suspensions comprising, consisting essentially of or consisting of the CMPs and/or CMP-TC conjugates of the present invention, are suitably used as or included in compositions for use in, or as, a medicament for treating, preventing or ameliorating a variety of diseases or disorders in humans or veterinary animals in need of treatment or prevention thereof. Other compositions provided by this aspect of the invention provide the use of CMPs conjugated to one or more diagnostic compounds or molecules, such as one or more labeled probes, which then are used as diagnostic reagents in a variety of tests and assays, particularly in vivo or in situ, to diagnose a disease, disorder, or physical condition in a human or veterinary animal. Such medicament compositions or diagnostic compositions may comprise, in addition to the CMPs, CMP-TC conjugates or CMPs conjugated to one or more diagnostic compounds or molecules, one or more additional therapeutic compounds or pharmaceutically active ingredients (e.g., one or more antibiotics, one or more growth factors, autologous plasma rich in growth factors (PRGF), one or more cytokines, one or more antibodies fragments thereof, one or more non-biologic small molecule therapeutic compounds, and pharmaceutically active salts, esters and derivatives thereof, and the like, including those described herein and others that are known in the art. The compositions of the invention may additionally or alternatively comprise one or more pharmaceutically acceptable carriers or excipients. Pharmaceutically acceptable carriers or excipients suitable for use in the compositions and methods of the invention include, for example, one or more solvents (which may include water, an organic solvent or an inorganic solvent), one or more buffers, one or more polymers, one or more salts, one or more sugars, one or more sugar alcohols, one or more disintegrating agents, one or more aerosolizing agents or carriers, one or more dessicants, and the like. Other pharmaceutically acceptable carriers or excipients suitable for use in the compositions of the present invention will be readily familiar to those of ordinary skill in the relevant arts.

Without wishing to be bound by theory, it is thought that the CMPs provided by the invention and used in the methods of the invention are useful in particular in repairing damaged collagen, particularly damaged helical collagen, that results from or that is involved in a variety of diseases, disorders, structural abnormalities, physical conditions and medical conditions in humans and veterinary animals. For example, when collagen is damaged structurally it is often hydrolyzed in at least one of the three helices forming triple helix collagen, thereby causing an unraveling and structural deformity of the triple helix. This disruption and digestion can also lead to collagen being fragmented and fractured into many smaller pieces which remain in the extracellular milieu or which find their way into the blood or lymphatic circulatory systems. Such fragments are ultimately either phagocytized or bound by scavenger cells, or bind to cell surface receptors on somatic cells in the human or veterinary animal. Such receptors (which may include, for example, integrins, discoidin-domain receptors, glycoprotein VI and leucocyte-associated immunoglobulin-like receptor-1 (LAIR-1)) control cellular functions such as growth, differentiation, morphogenesis, tissue repair, adhesion, migration, homeostasis, immune function and wound healing, are often disrupted, or their functions or signaling systems are up- or down-regulated, via the binding of such free collagen fragments. According to this theory, when CMPs encounter damaged collagen or fragments thereof they dynamically anneal to or bind the fractured collagen triple helix and structurally repair it, resulting in (among other things) the restoration of cellular receptors to their proper function and levels of signaling activity. Thus, in this way the aggregate result of the application of CMPs to a human or veterinary animal having a disease, disorder, structural abnormality or injury involving or resulting from damaged collagen is to unleash an accelerated wound healing process which in some physiological contexts includes rapid epithelial cell, endothelial cell or neural cell growth, migration and adhesion over the now repaired collagen matrix, resulting in the restoration of normal or near-normal structure and function of such cells, and tissues, organs and organ systems comprising such cells.

Diseases, disorders, physical conditions and medical conditions suitably treated, prevented, ameliorated or diagnosed using the compositions and methods of the invention include, but are not limited to ocular diseases or disorders, skin diseases or disorders, cancers, gastrointestinal diseases or disorders, genitourinary tract diseases or disorders, fibrotic diseases or disorders, cardiovascular diseases or disorders, bone diseases or disorders, rheumatic diseases or disorders and nerve or nervous system diseases or disorders. Suitable dosages of the compositions and conjugates of the invention for such uses are concentrations of the CMP component of the composition or conjugate at about 10 ng/ml to about 500 μg/ml, about 15 ng/ml to about 400 μg/ml, about 20 ng/ml to about 300 μg/ml, about 25 ng/ml to about 250 μg/ml, about 30 ng/ml to about 200 μg/ml, about 35 ng/ml to about 200 μg/ml, about 40 ng/ml to about 200 μg/ml, about 50 ng/ml to about 200 μg/ml, about 75 ng/ml to about 200 μg/ml, and about 100 ng/ml to about 200 μg/ml. In certain such embodiments, the conjugates or compositions are suitably applied to the tissue, organ or organ system being treated in dosages equivalent to a CMP concentration of about 25 μg/ml to about 500 μg/ml, e.g., about 25 μg/ml, about 30 μg/ml, about 35 μg/ml, about 40 μg/ml, about 45 μg/ml, about 50 μg/ml, about 75 μg/ml, about 100 μg/ml, about 125 μg/ml, about 150 μg/ml, about 175 μg/ml, about 200 μg/ml, about 225 μg/ml, about 250 μg/ml, about 300 μg/ml, about 350 μg/ml, about 400 μg/ml, about 450 μg/ml or about 500 μg/ml. In particular such embodiments, concentrations equivalent to a CMP concentration of between about 25 μg/ml, 50 μg/ml, 75 μg/ml or 100 μg/ml are used. Additional concentrations and amounts of the conjugates or compositions of the invention that are suitably used in such methods can be easily determined by one of ordinary skill, based on the information contained herein and that is available in the art, without the need to resort to undue experimentation.

Ocular diseases or disorders that can be treated, prevented, ameliorated or diagnosed using the compositions and methods of the invention include but are not limited to those involving the cornea and/or the sclera of the eye, i.e., corneoscleral diseases, disorders and conditions. Without wishing to be bound by theory, it is thought that the compositions of the invention, particularly the one or more CMPs contained in such compositions, repair disordered, disorganized or digested collagen found in the extracellular matrix and/or stroma of the cornea and sclera in such a way as to repair the collagen matrix/stroma and restore the homeostatic structure and function (including stiffening or strengthening) of the cornea and/or sclera, thereby treating or ameliorating such diseases and disorders and the signs/symptoms thereof. In other aspects, the compositions of the invention, particularly the one or more CMPs contained in such compositions, prevent the disorganization and digestion of the collagen in the extracellular matrix and/or stroma of the cornea and sclera upon insult or injury, in such a way as to preserve the collagen matrix/stroma and thus the homeostatic structure and function of the cornea and/or sclera, thereby preventing such diseases and disorders and the signs/symptoms thereof. Examples of corneoscleral diseases, disorders and conditions that are suitably treated, prevented, ameliorated or diagnosed using the compositions and methods of the invention include but are not limited to myopia, presbyopia, hyperopia, keratitis, episcleritis, scleritis, corneal ulceration, sequelae of corneal ulceration, corneal ectasia, acquired abnormalities of corneal shape, keratoconus, corneal astigmatism, keratoglobus, posterior corneal depressions, keratectasia, keratocele, descemetocele, pellucid marginal degeneration, Terrien's marginal dystrophy, Mooren's ulcers, central corneal ulcers, marginal corneal ulcers, staph marginal ulceration, Salzman's nodular dystrophy, age-related peripheral corneal atrophy, geographic ulceration, disciform stromal keratitis, metaherpetic ulceration, keratomalacia, post penetrating keratoplasty, incisional wounds, anterior membrane dystrophies, stromal dystrophies, ocular mucous membrane pemphigoid, necrotizing scleritis, scleromalacia, coloboma, scleral buckle induced scleromalacia, congenital hereditary stromal dystrophy, congenital anterior staphyloma, sclerocornea, traumatic breaks in Descemet's membrane, corneal keloids, scleral ectasia, scleral staphyloma, deep scleritis, necrotizing scleritis, scleromalacia perforans, hyaline degeneration of the sclera, paralimbal scleromalacia, ocular graft vs host disease, and choroideremia. In particular, the compositions and methods of the present invention are suitable for treating, ameliorating, preventing and/or diagnosing myopia, presbyopia, and keratoconus. Other corneoscleral diseases, disorders and conditions that may be suitably prevented, treated, ameliorated or diagnosed using the compositions and methods of the present invention will be apparent to one of ordinary skill in the art based on information readily available in the literature.

Other ocular diseases or disorders that can be treated, prevented, ameliorated or diagnosed using the compositions and methods of the invention include anterior segment diseases and disorders including but not limited to glaucoma, cataracts, vitreous adhesions or floaters, macular degeneration, dry eye syndrome (also known as dry eye disease), corneal keratitis, non-infectious corneal ulceration, non-infectious corneal melting, infectious corneal ulceration, infectious corneal melting, conjunctivitis, Stevens-Johnson Syndrome, iritis, uveitis, vitritis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, ectasia, corneal lacerations, corneal erosion, corneal abrasions, acute or chronic corneal pain (particularly that resulting from damage or injury to the corneal nerves or denervation; see, e.g., Rosenthal, P. and Borsook, D., Br J Ophthalmol. 2016; 100(1):128-134; Theophanous, C., et al., Optom. Vis. Sci. 2015; 92(9):e233-240; Belmonte, C., et al., Ocul. Surf 2004; 2(4):248-253; Belmonte, C., et al., Exp. Eye Res. 2004; 78(3):513-525; Belmonte, C., et al., Curr. Ophthalmol Rep. 2015; 3(2):111-121), including but not limited to paraocular pain, extraocular pain and post-herpetic neuralgia, and post-operative afflictions of the eye resulting from eye surgery. Such post-operative afflictions of the eye resulting from eye surgery can be, for example, afflictions arising post-operatively from cataract surgery or glaucoma surgery, particularly wherein those afflictions result in or are a post-operative state of the eye requiring medication.

Additional ocular diseases or disorders that can be treated, prevented, ameliorated or diagnosed using the compositions and methods of the invention include but are not limited to posterior segment diseases and disorders, particularly those involving the retina, including but not limited to macular degeneration (wet, dry and age-related), retinitis pigmentosa, retinal tears or detachment, retinopathy (e.g., diabetic retinopathy), arterial or venous occlusion (e.g., BRAO (Branch Retinal Artery Occlusion), CRAO (Central Retinal Artery Occlusion), BRVO (Branch Retinal Vein Occlusion) and CRVO (Central Retinal Vein Occlusion), optic neuritis, optic neuropathy (including, for example, AION (Anterior Ischemic Optic Neuropathy), and traumatic optic neuropathy), optic atrophy (e.g., glaucomatous optic atrophy), one or more neuropathies impacting the eye or area around the eye, including paraocular diseases, disorders or conditions and extraocular diseases, disorders or conditions, such as cranial nerve palsies including but not limited to Cranial III Nerve Palsy, Cranial Nerve IV Palsy, Cranial Nerve V Palsy (e.g., trigeminal neuralgia and post-herpes zoster neuralgia), Cranial Nerve VI Palsy and Cranial Nerve VII Palsy (e.g., Bell's Palsy)), and the like, and other retinal and posterior segment related disorders and diseases involving the retinal epithelium, particularly the retinal pigment epithelium, retinal blood vessels and/or retinal, cranial or optic nerves. Additional ocular disorders that are advantageously treated, ameliorated and/or prevented using the compositions and methods of the present invention include but are not limited to refractive eye disorders including myopia, presbyopia and amblyopia. For example, myopia is known to be associated with axial elongation of the eye, which may in part involve collagen disruption in the ocular sclera (see, e.g., Guo, P. et al., Trans. Vis. Sci. Tech. 9(9):45 (2020); Zhao, F. et al., Am. J. Pathol. 188:1754-1767 (2018)), and which may be ameliorated by scleral strengthening by cross-linking or other manipulation of the scleral connective tissue (see, e.g., Backhouse, S. et al., Ann. Eye Sci. 3:5 (2018); Grytz, R. et al., Curr. Opin. Biomed. Eng. 15:40-50 (2020); Garcia, M. B. Et al., Invest. Ophthalmol. Vis. Sci. 58:1875-1886 (2017)).

According to this aspect of the invention, methods of treating or preventing an ocular disease, disorder or wound in a human or veterinary animal suffering from or predisposed to an ocular disease, disorder or wound, comprise administering the compositions described herein, particularly the CMPs or CMP-TC conjugates and/or compositions comprising such conjugates, to an eye of a human or veterinary animal. Without wishing to be bound by theory, the inventors surmise that in areas of eye disease or disorder there is sufficient disruption of type I collagen such that the CMP will target the site of the eye disease or disorder specifically and intercalate into the collagen structure, for example by intercalating into one or more damaged helices of helical collagen, thereby directly reforming a functioning collagen helix or matrix or, in cases where the CMP is conjugated to a therapeutic compound, delivering the therapeutic compound to the site where it must act to treat, prevent or ameliorate the eye disease or disorder. In certain such anterior segment ocular diseases or disorders, such as acute or chronic corneal pain (including, but not limited to paraocular pain, extraocular pain, and post-herpetic neuralgia), denervated corneas suffer from poor healing capability and as such a topical therapy which can impact neuroregeneration would be a welcomed therapy in this area. Pain, both acute as well as chronic, is mediated by damaged corneal nerves (see, e.g., Rosenthal, P. and Borsook, D., Br J Ophthalmol. 2016; 100(1):128-134; Theophanous, C., et al., Optom. Vis. Sci. 2015; 92(9):e233-240; Belmonte, C., et al., Ocul. Surf 2004; 2(4):248-253; Belmonte, C., et al., Exp. Eye Res. 2004; 78(3):513-525; Belmonte, C., et al., Curr. Ophthalmol Rep. 2015; 3(2):111-121), and thus a therapeutic which could be beneficial to nerve health would be clinically valuable for such patients. Based on the findings described herein relating to the behavior of dorsal root ganglion cells when exposed to a CMP of the invention (SEQ ID NO:1) after damage to a collagen support layer (see Example 4 hereinbelow), it can be expected that any cranial nerve would behave in a similar way. Corneal nerves, as branches of the trigeminal nerve, will therefore benefit from a therapy which includes the administration of one or more of the CMPs or CMP-TC conjugates described herein topically to the cornea. With a repaired and regenerated nerve, corneal recovery and pain relief would then follow, resulting in the amelioration of the acute or chronic corneal pain.

The conjugates or compositions are suitably applied to the eye in a dosage sufficient to treat or prevent the ocular disease, disorder or wound, and the condition of the eye in said human or veterinary animal is then monitored over time for improvement in the disease state or physical condition. If necessary, the conjugate or composition of the invention is then periodically readministered to the eye, according to dosing and treatment schedules and protocols described herein and others that will be familiar to the ordinarily skilled artisan, until the ocular disease, disorder or wound is cured, prevented or ameliorated. In such embodiments, the conjugates or compositions of the invention for treatment of anterior segment diseases and disorders can be suitably administered to the eye to the surface of the eye, conjunctivally, or subconjunctivally, particularly by administering the conjugate or composition dropwise onto the surface of the eye or into the subconjunctival fornix. In other embodiments, the conjugates or compositions of the invention for treatment of anterior segment diseases and disorders can be suitably administered to the front of the eye, including the sclera and the vitreous, either dropwise (relying on the ability of the CMP-containing formulations to migrate across the surface epithelium) or via injection, e.g., via intravitreal injection, according to methods that are well-known to those of ordinary skill in the medical and pharmaceutical arts. In other embodiments involving treatment, prevention, cure or diagnosis of posterior segment diseases and disorders, the conjugates and compositions of the invention can be administered to the posterior segment, e.g., at or near the retina, via mechanical introduction such as via injection using a needle or other suitable apparatus, or by administration of the conjugate or composition to the surface of the eye in the form of drops, in which the conjugate or composition (or component thereof, e.g., a CMP or CMP-TC conjugate) is transported or migrates to the posterior segment of the eye (e.g., at or near the retina). Administration of the conjugates or compositions to the eye can be accomplished by any well-known means, including applying the conjugates or compositions to the eye in the form of one or more drops or aliquots of a solution, a gel or a suspension that contains the composition or conjugates; via injection; in the form of a solid material such as a wafer or film (such as those described herein) that is implanted into an eye structure; in the form of a mesh or patch; by attaching the conjugate or composition to, or enclosing it within, one or more gels, spheres or nanoparticles that are then delivered into an eye structure. Other suitable methods of applying the conjugates or compositions to the eye to accomplish the therapeutic and diagnostic methods of the invention will be readily apparent to the ordinarily skilled artisan.

Skin diseases or disorders that can be treated, prevented, ameliorated or diagnosed using the compositions and methods of the invention include but are not limited to skin wounds, scarring, wrinkles, “crepey skin”, skin cancer (e.g., melanomas, skin carcinomas, skin sarcomas, histiocytomas) and skin burns, including sunburn. Other skin diseases or disorders suitably treated, prevented, ameliorated or diagnosed according to the invention include psoriasis and eczema, shingles, irritant contact dermatitis and allergic contact dermatitis (such as poison ivy, poison oak or poison sumac).

According to this aspect of the invention, methods of treating or preventing a skin disease, disorder or wound in a human or veterinary animal suffering from or predisposed to a skin disease, disorder or wound, comprise administering the compositions described herein, particularly the CMPs and CMP-TC conjugates, and compositions comprising such CMPs and CMP-TC conjugates, to the skin of a human or veterinary animal at a site proximal to the location of a lesion associated with or causing the skin disease, wound or disorder. Without wishing to be bound by theory, the inventors surmise that in areas of skin disease or disorder there is sufficient disruption of type I collagen such that the CMP will target the site of the skin disease or disorder specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the skin disease or disorder. Alternatively, the disease or disorder afflicting the skin can be excised or resected from the skin (e.g., via surgical removal, for example of a skin cancer), and the skin wound resulting from such excision or resection can be treated with one or more compositions of the invention according to the methods described herein. In certain embodiments, one or more of the CMPs themselves, or one or more CMP-TC conjugates, or any combination thereof, can be introduced into the skin, particularly intraepidermally, intradermally or subcutaneously, in the form of a so-called “cosmeceutical” (see, e.g., Epstein, H., Clin. Dermatol. 27(5):453-460 (2009)). Particularly preferred CMP-TC conjugates or compositions for use in such aspects of the invention include those wherein the therapeutic compound is Substance P (SEQ ID NO:389), particularly those wherein the CMP-TC conjugate has an amino acid sequence corresponding to any one of SEQ ID NOs: 390-396. Additional particularly preferred CMP-TC conjugates or compositions for use in such aspects of the invention include those wherein the therapeutic compound is retinol or a derivative or precursor thereof. Additional preferred compositions comprise such compositions that comprise or further comprise at least one growth factor, at least one antibiotic, at least one antifungal compound or at least one antiviral compound. Suitable growth factors, antibiotics, antifungal compounds and antiviral compounds include those described herein and others that are well-known in the dermatological and other relevant arts. According to this aspect of the invention, the conjugates or compositions are suitably applied to or into the skin in a dosage sufficient to treat or prevent the skin disease, disorder or wound, and the condition of the skin in said human or veterinary animal is then monitored over time for improvement in the disease state or physical condition. If necessary, the conjugate or composition of the invention is then periodically readministered to or into the skin, according to dosing and treatment schedules and protocols described herein and others that will be familiar to the ordinarily skilled artisan, until the skin disease, disorder or wound is cured, prevented or ameliorated. In such embodiments, the conjugates or compositions of the invention are suitably administered to or into the skin topically, intraepidermally, intradermally or subdermally. Administration of the conjugates or compositions to or into the skin can be accomplished by any well-known means, including in the form of a solution, an ointment, a salve, a patch, a cream, a topical solution and a drug eluting wafer. For example, the conjugates or compositions can be applied to or introduced into the skin in the form of one or more drops of solution or a suspension that contains the composition or conjugates; via injection; in the form of a coating on a solid material that is implanted into the skin; in the form of a mesh or patch; by attaching the conjugate or composition to, or enclosing it within, one or more nanoparticles that are then delivered into the skin. Other suitable methods of applying the conjugates or compositions to or into the skin to accomplish the therapeutic and diagnostic methods of the invention will be readily apparent to the ordinarily skilled artisan.

Cancers that can be treated, prevented, ameliorated or diagnosed using the compositions and methods of the invention include but are not limited to skin cancers (e.g., those described elsewhere herein), intraluminal cancers and brain cancers. Intraluminal cancers suitably treated, prevented, diagnosed or ameliorated using the conjugates, compositions and methods of the invention include but are not limited to colorectal cancer, intestinal cancer, duodenal cancer, stomach cancer, pancreatic cancer, esophageal cancer, a bladder cancer (e.g., non-muscle-invasive bladder cancer or carcinoma in situ of the bladder), a cancer of the upper urinary tract, alternatively referred to and also known to those of ordinary skill as the renal pelvis (e.g., upper tract urothelial carcinoma, Wilms tumor and renal cancer), vaginal cancer, cervical cancer, uterine cancer, ovarian cancer, luminal breast cancer and lung cancer. Brain cancers suitably treated, prevented, diagnosed or ameliorated using the conjugates, compositions and methods of the invention include but are not limited to gliomas, glioblastomas, meningiomas, pituitary tumors, craniopharyngioma and hemangioblastomas. Other non-luminal cancers are also suitably treated, prevented, diagnosed or ameliorated using the conjugates, compositions and methods of the invention, including but not limited to prostate cancer, testicular cancer, non-luminal breast cancer, bone cancer, head and neck cancer, thyroid cancer, liver cancer, sarcomas (e.g., Kaposi sarcoma, Ewing sarcoma, osteosarcoma, soft tissue sarcoma and rhabdomyosarcoma), and the like.

According to this aspect of the invention, methods of treating or preventing a cancer in a human or veterinary animal suffering from or predisposed to a cancer, comprise administering the compositions described herein, particularly the CMPs and CMPs and/or conjugates, into the organ lumen, or into the cranium or into or on the brain, of a human or veterinary animal, at a site proximal to the location of the cancer or tumor. Without wishing to be bound by theory, the inventors surmise that in areas of cancer there is sufficient disruption of type I collagen, or upregulation of type I collagen in the case of brain cancer, such that the CMP will target the site of the cancer specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the cancer. Particularly preferred conjugates or compositions for use in this aspect of the invention include those wherein the therapeutic compound is a biologic therapeutic compound, particularly one or more monoclonal antibodies or fragments thereof or one or more therapeutic fusion proteins, particularly recombinant fusion proteins, including those described herein. Additional preferred compositions comprise such compositions that further comprise at least one growth factor, at least one antibiotic, at least one antifungal compound or at least one antiviral compound. Suitable growth factors, antibiotics, antifungal compounds and antiviral compounds include those described herein and others that are well-known in the dermatological and other relevant arts. According to this aspect of the invention, the conjugates or compositions are suitably applied to or into the organ lumen, or the cranium or brain, in a dosage sufficient to treat, prevent or ameliorate the cancer, and the progression, remission or stasis of the cancer in the human or veterinary animal is then monitored over time for improvement in the cancer disease state (e.g., shrinkage of the tumor or at least non-progression or remission of the cancer). If necessary, the conjugate or composition of the invention is then periodically readministered into the organ lumen, or into the cranium or into or on the brain, according to dosing and treatment schedules and protocols described herein and others that will be familiar to the ordinarily skilled artisan, until the cancer is cured, prevented or ameliorated, or goes into permanent remission. In such embodiments, the conjugates or compositions of the invention are suitably administered to or into the organ lumen or the brain parenterally or via direct application to the tumor site or, in the case of excision or resection of the tumor, via direct application to the tumor bed or the wound remaining following excision or resection of the tumor. Parenteral administration of the conjugates or compositions of the invention can be accomplished via a route selected from the group consisting of subcutaneous injection, intravenous infusion, intraarterial infusion, transdermal diffusion, implantation of a drug eluting wafer or film, sublingually, orally, via aerosol inhalation, intravaginally, rectally, or intracranially. In certain such embodiments the conjugate or composition can be administered parenterally to the human or veterinary animal in the form of a mesh, film, wafer, sphere, nanoparticle, gel or patch that is implanted into the human or veterinary animal at or proximal to the site of the cancer. In other such embodiments, particularly those in which the cancer is an intraluminal cancer, the conjugates or compositions of the invention can be administered to the lumen of the cancerous organ in the human or veterinary animal using a medical instrument suitable for such purpose, such as an endoscope, a bronchoscope (for example, via bronchial lavage for treating, preventing or diagnosing a cancer of the pulmonary tract such as bronchial cancer or lung cancer), a proctoscope, a colonoscope, a cystoscope (e.g., into the bladder or upper urinary tract via cystoscopic irrigation), a gastroscope and a laparoscope, or other suitable surgical/medical instruments capable of delivering a dose of a medicament such as the conjugates and compositions of the invention to the human or veterinary animal at the site of the cancer. In certain such embodiments, the conjugate or composition can be administered following surgical excision or resection of a solid tumor, or removal or aspiration of a tumor ascites using, e.g., a trochar introduced into the abdomen for removal of abdominal ascites fluid. In such embodiments, the conjugate or composition of the invention (along with, optionally, one or more additional therapeutic agents) can be introduced directly into the surgical excision or into the ascites area, for example through any of the instruments or devices described above.

In other embodiments, administration of the conjugates or compositions to or into the organ lumen or the brain can be accomplished by any well-known means, including in the form of a solution, an ointment, a salve, a patch, a cream, a topical solution and a drug eluting wafer. For example, the conjugates or compositions can be applied to or introduced into the lumen of the organ or into or on the brain in the form of one or more drops of solution or a suspension that contains the composition or conjugates; via injection; in the form of a coating on a solid material that is implanted into the organ lumen or the brain; in the form of a mesh or patch; by attaching the conjugate or composition to, or enclosing it within, one or more nanoparticles that are then delivered into the organ lumen or the brain. Other suitable methods of applying the conjugates or compositions to or into the organ lumen or the brain to accomplish the therapeutic and diagnostic methods of the invention will be readily apparent to the ordinarily skilled artisan.

Gastrointestinal diseases or disorders that can be treated, prevented, ameliorated or diagnosed using the compositions and methods of the invention include but are not limited to irritable bowel syndrome, Crohn's Disease, an ulcer, ulcerative colitis, esophagitis, Barrett's esophagitis, gastritis and proctitis.

According to this aspect of the invention, methods of treating or preventing a gastrointestinal disease or disorder in a human or veterinary animal suffering from or predisposed to a gastrointestinal disease or disorder comprise administering the compositions described herein, particularly the CMPs and CMP-TC conjugates and compositions comprising such CMPs and/or conjugates, into the gastrointestinal tract of a human or veterinary animal, at a site proximal to the location of a lesion associated with or causing the gastrointestinal disease or disorder. Without wishing to be bound by theory, the inventors surmise that in areas of certain gastrointestinal diseases and disorders there is sufficient disruption of type I collagen such that the CMP will target the site of the gastrointestinal disease or disorder specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the gastrointestinal disease or disorder. Particularly preferred conjugates or compositions for use in this aspect of the invention include those wherein the therapeutic compound is a biologic therapeutic compound, particularly one or more monoclonal antibodies or fragments thereof or one or more therapeutic fusion proteins, particularly recombinant fusion proteins, including those described herein. According to this aspect of the invention, the conjugates or compositions are suitably applied to or into the gastrointestinal tract in a dosage sufficient to treat, prevent or ameliorate the gastrointestinal disease or disorder, and the progression, remission or stasis of the gastrointestinal disease or disorder in the human or veterinary animal is then monitored over time for improvement in the disease or disorder state. If necessary, the conjugate or composition of the invention is then periodically readministered into the gastrointestinal tract according to dosing and treatment schedules and protocols described herein and others that will be familiar to the ordinarily skilled artisan, until the gastrointestinal disease or disorder is cured, prevented or ameliorated. In such embodiments, the conjugates or compositions of the invention are suitably administered to or into the gastrointestinal tract parenterally or topically. Parenteral administration is accomplished by any art-known route of administration of a therapy to the gastrointestinal tract, for example via a route selected from the group consisting of subcutaneous injection, intravenous infusion, intraarterial infusion, transdermal diffusion, implantation of a drug eluting wafer, sublingually, orally or rectally. In such methods, the composition is suitably administered parenterally to the human or veterinary animal in the form of a pill, capsule, solution, suspension or powder that is ingested by the human or veterinary animal, or in the form of a mesh or patch that is implanted within the gastrointestinal tract at or proximal to the site of the disease or disorder. In other such embodiments, particularly those in which the disease or disorder is intraluminal in the gastrointestinal tract, the conjugates or compositions of the invention can be administered to the lumen of the gastrointestinal organ in the human or veterinary animal using a medical instrument suitable for such purpose, such as a proctoscope, a colonoscope, a cystoscope (e.g., into the bladder or upper urinary tract cystoscopically), a gastroscope and a laparoscope, or other suitable surgical/medical instruments capable of delivering a dose of a medicament such as the conjugates and compositions of the invention to the human or veterinary animal at the site of the gastrointestinal disease or disorder.

In other embodiments, administration of the conjugates or compositions to or into the gastrointestinal tract can be accomplished by any well-known means, including in the form of a solution, an ointment, a salve, a patch, a cream, a topical solution and a drug eluting wafer. For example, the conjugates or compositions can be applied to or introduced into the gastrointestinal tract in the form of one or more drops of solution or a suspension that contains the composition or conjugates; via injection; in the form of a coating on a solid material that is implanted into the gastrointestinal tract; in the form of a mesh or patch; by attaching the conjugate or composition to, or enclosing it within, one or more nanoparticles that are then delivered into the gastrointestinal tract. Other suitable methods of applying the conjugates or compositions to or into the gastrointestinal tract to accomplish the therapeutic and diagnostic methods of the invention will be readily apparent to the ordinarily skilled artisan.

Genitourinary diseases or disorders that can be treated, prevented, ameliorated or diagnosed using the compositions and methods of the invention include but are not limited to female urinary incontinence, cystitis, interstitial cystitis, irritable bladder syndrome, ureteritis and vaginitis.

According to this aspect of the invention, methods of treating or preventing a genitourinary disease or disorder in a human or veterinary animal suffering from or predisposed to a genitourinary disease or disorder comprise administering the compositions described herein, particularly the CMPs and CMP-TC conjugates and compositions comprising such CMPs and/or conjugates, into the genitourinary tract of a human or veterinary animal, at a site proximal to the location of a lesion associated with or causing the genitourinary tract disease or disorder. Without wishing to be bound by theory, the inventors surmise that in areas of certain genitourinary diseases and disorders there is sufficient disruption of type I collagen such that the CMP will target the site of the genitourinary disease or disorder specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the genitourinary disease or disorder. According to this aspect of the invention, the conjugates or compositions are suitably applied to or into the genitourinary tract in a dosage sufficient to treat, prevent or ameliorate the genitourinary disease or disorder, and the progression, remission or stasis of the genitourinary disease or disorder in the human or veterinary animal is then monitored over time for improvement in the disease or disorder state. If necessary, the conjugate or composition of the invention is then periodically readministered into the genitourinary tract according to dosing and treatment schedules and protocols described herein and others that will be familiar to the ordinarily skilled artisan, until the genitourinary disease or disorder is cured, prevented or ameliorated. In such embodiments, the conjugates or compositions of the invention are suitably administered to or into the genitourinary tract parenterally or topically. Parenteral administration is accomplished by any art-known route of administration of a therapy to the gastrointestinal tract, for example via a route selected from the group consisting of subcutaneous injection, intravenous infusion, intraarterial infusion, transdermal diffusion, implantation of a drug eluting wafer, sublingually, orally, vaginally or rectally. In such methods, the composition is suitably administered parenterally to the human or veterinary animal in the form of a pill, capsule, solution, suspension or powder that is ingested by the human or veterinary animal, or in the form of a mesh or patch that is implanted within the genitourinary tract at or proximal to the site of the disease or disorder. In other such embodiments, particularly those in which the disease or disorder is intraluminal in the gastrointestinal tract, the conjugates or compositions of the invention can be administered to the lumen of the genitourinary organ in the human or veterinary animal using a medical instrument suitable for such purpose, such as an endoscope, a vaginoscope, and a laparoscope, or other suitable surgical/medical instruments capable of delivering a dose of a medicament such as the conjugates and compositions of the invention to the human or veterinary animal at the site of the genitourinary disease or disorder.

In other embodiments, administration of the conjugates or compositions to or into the genitourinary tract can be accomplished by any well-known means, including in the form of a solution, an ointment, a salve, a patch, a wafer, a film, a gel, spheres, nanoparticles, a cream, a topical solution and a drug eluting wafer. For example, the conjugates or compositions can be applied to or introduced into the genitourinary tract in the form of one or more drops of solution or a suspension that contains the composition or conjugates; via injection; in the form of a coating on a solid material that is implanted into the genitourinary tract; in the form of a mesh or patch; by attaching the conjugate or composition to, or enclosing it within, one or more nanoparticles that are then delivered into the genitourinary tract. Other suitable methods of applying the conjugates or compositions to or into the genitourinary tract to accomplish the therapeutic and diagnostic methods of the invention will be readily apparent to the ordinarily skilled artisan.

Fibrotic diseases or disorders that can be treated, prevented, ameliorated or diagnosed using the compositions and methods of the invention include but are not limited to pulmonary fibrosis, liver cirrhosis, myocardial fibrosis, surgical scarring, systemic sclerosis, scleroderma, keloid formation, proliferative vitreo retinopathy, and the like.

According to this aspect of the invention, methods of treating or preventing a fibrotic disease or disorder in a human or veterinary animal suffering from or predisposed to a fibrotic disease or disorder comprise administering the compositions described herein, particularly the CMPs and CMP-TC conjugates and compositions comprising such CMPs and/or conjugates, into or near one or more tissues, organs or organ systems of a human or veterinary animal, at a site proximal to the location of a fibrotic lesion associated with or causing the fibrotic disease or disorder. Without wishing to be bound by theory, the inventors surmise that in areas of certain fibrotic diseases and disorders there is sufficient disruption of type I collagen such that the CMP will target the site of the fibrotic disease or disorder specifically and intercalate into the collagen structure, thereby directly reforming a functioning collagen matrix or, in cases where the CMP is conjugated to a therapeutic compound, thereby delivering the therapeutic compound to the site where it must act to treat, prevent or ameliorate the fibrotic disease or disorder. According to this aspect of the invention, the conjugates or compositions are suitably applied to, near or into the tissue, organ or organ system in a dosage sufficient to treat, prevent or ameliorate the fibrotic disease or disorder, and the progression, remission or stasis of the fibrotic disease or disorder in the human or veterinary animal is then monitored over time for improvement in the disease or disorder state. If necessary, the conjugate or composition of the invention is then periodically readministered into, near or onto one or more tissues, organs or organ systems according to dosing and treatment schedules and protocols described herein and that will be familiar to the ordinarily skilled artisan, until the fibrotic disease or disorder is cured, prevented or ameliorated. In such embodiments, the conjugates or compositions of the invention are suitably administered to, near, on or into the tissues, organs or organ systems parenterally or topically. Parenteral administration is accomplished by any art-known route of administration of a therapy to the tissues, organ or organ systems, for example via a route selected from the group consisting of subcutaneous injection, intravenous infusion, intraarterial infusion, endoscopic application, transdermal diffusion, implantation of a drug eluting wafer, film, gel or putty, sublingually, orally or rectally. In such methods, the composition is suitably administered parenterally to the human or veterinary animal in the form of a pill, capsule, solution, suspension or powder that is ingested by the human or veterinary animal, or in the form of a mesh, film, wafer, gel, sphere, nanoparticle, putty or patch that is implanted near, on or into the fibrotic tissue, organ or organ system at or proximal to the site of the disease or disorder.

In other embodiments, administration of the conjugates or compositions to, near or into the tissues, organs or organ systems can be accomplished by any well-known means, including in the form of a solution, an ointment, a salve, a patch, a film, a gel, spheres, nanoparticles, putty, a cream, a topical solution and a drug eluting wafer. For example, the conjugates or compositions can be applied to or near, or introduced into, the tissues, organs or organ systems in the form of one or more drops of solution or a suspension that contains the composition or conjugates; via injection; in the form of a coating on a solid material that is implanted into, near or onto the tissues, organs or organ systems; in the form of a mesh or patch; by attaching the conjugate or composition to, or enclosing it within, one or more nanoparticles that are then delivered into, near or on the tissues, organs or organ systems. Other suitable methods of applying the conjugates or compositions to, on, near or into the tissues, organs or organ systems to accomplish the therapeutic and diagnostic methods of the invention will be readily apparent to the ordinarily skilled artisan.

Cardiovascular diseases or disorders that can be treated, prevented, ameliorated or diagnosed using the compositions and methods of the invention include but are not limited to myocardial infarction, cardiac insufficiency, cardiac valve disorders, atherosclerosis, cardiomyophathy, arrhythmias, congenital heart disease, coronary artery disease, pericardial disease, vascular occlusive disease (e.g., affecting the carotid artery, the aorta, the renal artery, the femoral artery, the pulmonary artery, and other large vessels and small vessels which may be arteries, arterioles, veins, venules and the like), Marfan syndrome, and the like.

According to this aspect of the invention, methods of treating or preventing a cardiovascular disease or disorder in a human or veterinary animal suffering from or predisposed to a cardiovascular disease or disorder comprise administering the compositions described herein, particularly the CMPs and/or CMP-TC conjugates and compositions comprising such CMPs and/or conjugates, into the vascular system of a human or veterinary animal suffering from or predisposed to such a disease or disorder. Without wishing to be bound by theory, the inventors surmise that in areas of certain cardiovascular diseases and disorders there is sufficient disruption of type I collagen such that the CMP introduced into the vascular system of the subject will target the site of the cardiovascular disease or disorder specifically and intercalate into the collagen structure, thereby directly reforming a functioning collagen matrix or, in cases where the CMP is conjugated to a therapeutic compound, thereby delivering the CMP and/or therapeutic compound to the site where it must act to treat, prevent or ameliorate the cardiovascular disease or disorder. According to this aspect of the invention, the conjugates or compositions are suitably applied to or into the vascular system in a dosage sufficient to treat, prevent or ameliorate the cardiovascular disease or disorder, and the progression, remission or stasis of the cardiovascular disease or disorder in the human or veterinary animal is then monitored over time for improvement in the disease or disorder state. If necessary, the conjugate or composition of the invention is then periodically readministered into the vascular system according to dosing and treatment schedules and protocols described herein and others that will be familiar to the ordinarily skilled artisan, until the cardiovascular disease or disorder is cured, prevented or ameliorated. In such embodiments, the conjugates or compositions of the invention are suitably administered to or into the heart, pericardium, vessel or other relevant component of the vascular system parenterally or topically. Parenteral administration is accomplished by any art-known route of administration of a therapy to the vascular system, for example via a route selected from the group consisting of subcutaneous injection, intravenous infusion, intraarterial infusion, transdermal diffusion, via catheterization, embolization, implantation of a drug eluting wafer or film, sublingually, orally, rectally. In such methods, the composition is suitably administered parenterally to the human or veterinary animal in the form of a pill, capsule, solution, suspension or powder that is ingested by the human or veterinary animal, or in the form of a mesh, wafer, film, gel, putty, sphere, nanoparticle or patch that is implanted within the heart, pericardium, vessel or other relevant component of the vascular system at or proximal to the site involved in the cardiovascular disease or disorder.

In other embodiments, administration of the conjugates or compositions to or into the vascular system can be accomplished by any well-known means, including in the form of a solution, an ointment, a salve, a patch, a film, a gel, spheres, nanoparticles, a cream, a topical solution and a drug eluting wafer. For example, the conjugates or compositions can be applied to or introduced into the heart, pericardium, vessel or other relevant component of the vascular system in the form of one or more drops of solution or a suspension that contains the composition or conjugates; via injection; in the form of a coating on a solid material that is implanted into the heart, pericardium, vessel or other relevant component of the vascular system; in the form of a mesh or patch; by attaching the conjugate or composition to, or enclosing it within, one or more nanoparticles that are then delivered into the heart, pericardium, vessel or other relevant component of the vascular system. Other suitable methods of applying the conjugates or compositions to or into the vascular system to accomplish the therapeutic and diagnostic methods of the invention will be readily apparent to the ordinarily skilled artisan.

Bone diseases or disorders that can be treated, prevented, ameliorated or diagnosed using the compositions and methods of the invention include but are not limited to osteoporosis, bone fracture, osteomyelitis, osteogenesis imperfecta, Paget disease of bone, osteonecrosis, rickets, osteomalacia, acromegaly and the like.

According to this aspect of the invention, methods of treating or preventing a bone disease or disorder in a human or veterinary animal suffering from or predisposed to a bone disease or disorder comprise administering the compositions described herein, particularly the CMPs and CMP-TC conjugates and compositions comprising such CMPs and/or conjugates, into or near one or more bones of a human or veterinary animal, at a site proximal to the location of a lesion associated with or causing the bone disease or disorder. Without wishing to be bound by theory, the inventors surmise that in areas of certain bone diseases and disorders there is sufficient disruption of type I collagen such that the CMP will target the site of the bone disease or disorder specifically and intercalate into the collagen structure, thereby directly reforming a functioning collagen matrix or, in cases where the CMP is conjugated to a therapeutic compound, thereby delivering the therapeutic compound to the site where it must act to treat, prevent or ameliorate the bone disease or disorder. According to this aspect of the invention, the conjugates or compositions are suitably applied to, near or into the bone in a dosage sufficient to treat, prevent or ameliorate the bone disease or disorder, and the progression, remission or stasis of the bone disease or disorder in the human or veterinary animal is then monitored over time for improvement in the disease or disorder state. If necessary, the conjugate or composition of the invention is then periodically readministered into, near or onto one or more bones according to dosing and treatment schedules and protocols described herein and others that will be familiar to the ordinarily skilled artisan, until the bone disease or disorder is cured, prevented or ameliorated. In such embodiments, the conjugates or compositions of the invention are suitably administered to, near, on or into the bones parenterally or topically. Parenteral administration is accomplished by any art-known route of administration of a therapy to the bones, for example via a route selected from the group consisting of subcutaneous injection, intravenous infusion, intraarterial infusion, endoscopic application, transdermal diffusion, implantation of a drug eluting wafer, film, gel or putty, sublingually, orally or rectally. In such methods, the composition is suitably administered parenterally to the human or veterinary animal in the form of a pill, capsule, solution, suspension or powder that is ingested by the human or veterinary animal, or in the form of a mesh, film, wafer, gel, sphere, nanoparticle, putty or patch that is implanted near, on or into the bone at or proximal to the site of the disease or disorder.

In other embodiments, administration of the conjugates or compositions to, near or into the bones can be accomplished by any well-known means, including in the form of a solution, an ointment, a salve, a patch, a film, a gel, spheres, nanoparticles, putty, a cream, a topical solution and a drug eluting wafer. For example, the conjugates or compositions can be applied to or near, or introduced into, the bones in the form of one or more drops of solution or a suspension that contains the composition or conjugates; via injection; in the form of a coating on a solid material that is implanted into, near or onto the bones; in the form of a mesh or patch; by attaching the conjugate or composition to, or enclosing it within, one or more nanoparticles that are then delivered into, near or on the bones. Other suitable methods of applying the conjugates or compositions to, on, near or into the bones to accomplish the therapeutic and diagnostic methods of the invention will be readily apparent to the ordinarily skilled artisan.

Rheumatic diseases or disorders that can be treated, prevented, ameliorated or diagnosed using the compositions and methods of the invention include but are not limited to arthritis (particularly rheumatoid arthritis, osteoarthritis and psoriatic arthritis), bursitis, crepitus, spondylosis, scleroderma, polymyalgia rheumatica and anarthritic syndrome.

According to this aspect of the invention, methods of treating or preventing a rheumatic disease or disorder in a human or veterinary animal suffering from or predisposed to a rheumatic disease or disorder comprise administering the compositions described herein, particularly the CMPs or CMP-TC conjugates and compositions comprising such CMPs and/or conjugates, to the human or veterinary animal at a site proximal to the location of a lesion associated with or causing the rheumatic disease or disorder. Without wishing to be bound by theory, the inventors surmise that in areas of certain rheumatic diseases and disorders there is sufficient disruption of type I collagen such that the CMP will target the site of the rheumatic disease or disorder specifically and intercalate into the collagen structure, thereby directly reforming a functioning collagen matrix or, in cases where the CMP is conjugated to a therapeutic compound, thereby delivering the therapeutic compound to the site where it must act to treat, prevent or ameliorate the rheumatic disease or disorder. According to this aspect of the invention, the conjugates or compositions are suitably applied to or into the human or veterinary animal in a dosage sufficient to treat, prevent or ameliorate the rheumatic disease or disorder, and the progression, remission or stasis of the rheumatic disease or disorder in the human or veterinary animal is then monitored over time for improvement in the disease or disorder state. If necessary, the conjugate or composition of the invention is then periodically readministered to the human or veterinary animal according to dosing and treatment schedules and protocols described herein and others that will be familiar to the ordinarily skilled artisan, until the rheumatic disease or disorder is cured, prevented or ameliorated. In such embodiments, the conjugates or compositions of the invention are suitably administered to or into the human or veterinary animal parenterally or topically. Parenteral administration is accomplished by any art-known route of administration of a therapy designed to treat, prevent or ameliorate a rheumatic disease or disorder, for example via a route selected from the group consisting of subcutaneous injection, intravenous infusion, intraarterial infusion, transdermal diffusion, implantation of a drug eluting wafer, sublingually, orally, vaginally or rectally. In such methods, the composition is suitably administered parenterally to the human or veterinary animal in the form of a pill, capsule, solution, suspension or powder that is ingested by the human or veterinary animal, or in the form of a mesh or patch that is implanted within the human or veterinary animal at or proximal to the site of the disease or disorder. In other such embodiments, particularly those in which the rheumatic disease or disorder is located in or near a bone, tendon, cartilage, ligament, bursa, joint or associated structure, the compositions or conjugates of the invention are suitably administered to the human or veterinary animal using a medical instrument suitable for such purpose, such as an laparoscope, or other suitable surgical/medical instruments capable of delivering a dose of a medicament such as the conjugates and compositions of the invention to the human or veterinary animal at the site of the genitourinary disease or disorder.

In other embodiments, administration of the conjugates or compositions to or into the human or veterinary animal can be accomplished by any well-known means, including in the form of a solution, an ointment, a salve, a patch, a cream, a topical solution and a drug eluting wafer. For example, the conjugates or compositions can be applied to or introduced into the human or veterinary animal in the form of one or more drops of solution or a suspension that contains the composition or conjugates; via injection; in the form of a coating on a solid material that is implanted into the human or veterinary animal; in the form of a mesh or patch; by attaching the conjugate or composition to, or enclosing it within, one or more nanoparticles that are then delivered into the human or veterinary animal. Other suitable methods of applying the conjugates or compositions to or into the human or veterinary animal to accomplish the therapeutic and diagnostic methods of the invention will be readily apparent to the ordinarily skilled artisan.

Nerve or nervous system (including the central nervous system (“CNS”) and peripheral nervous system (“PNS”) diseases or disorders that can be treated, prevented, ameliorated or diagnosed using the compositions and methods of the invention include but are not limited to injuries to one or more nerves or nerve processes (including axons, dendrites and neurons or neuronal bodies, ganglia, nerve bundles and the like), neurodegeneration (in many different physiological or disease contexts such as multiple sclerosis, amyotrophic lateral sclerosis, Parkinson's disease, Alzheimer's disease, Huntington's disease, a traumatic encephalopathy, a non-Alzheimer's dementia, encephalitis, meningitis, and the like), disorders involving peripheral nerves (such as diabetic peripheral neuropathy, nutritional neuropathy and alcohol-induced neuropathy), or certain neuroocular diseases and disorders including those involving or affecting the corneal nerves, retinal nerves and optic nerve, including but not limited to glaucoma, macular degeneration (wet and/or dry, which may or may not be age-related), neurotrophic keratitis, retinopathies (which may include diabetic retinopathy, ischemic retinopathy, a proliferative retinopathy, geographic atrophy, and other genetic-based retinopathies and genetic retinal diseases or disorders known in the art), damage to or inflammation of one or more corneal nerves (which may arise via damage to or inflammation of the eye via external diseases or trauma/wounding, including a transection of, or crush injury or torsional injury to, a nerve or nerve process), corneal pain (which may be acute or chronic, and which may result from damage or injury to the corneal nerves or corneal denervation, e.g., paraocular pain, extraocular pain, and post-herpetic neuralgia), an encephalopathy (e.g., traumatic encephalopathy such as concussion, encephalitis, meningitis), and the like. In certain such embodiments, the compositions and methods of the invention can be used to induce nerve repair or regrowth (e.g., via neuroregeneration), particularly in the cranial nerves including but not limited to the optic nerve, the retinal nerves, the acoustic nerve or the spinal nerve. In other such embodiments, the compositions and methods of the invention can be used to protect certain nerves from degeneration, or from further or continued degeneration (i.e., provide a neuroprotective function), which may, for example, be useful in preventing, reducing or slowing the progression of degeneration of the peripheral nerves for the prevention and/or treatment of diabetic peripheral neuropathy, nutritional neuropathy and alcohol-induced peripheral neuropathy, as well as of the corneal nerves, optic nerve and/or the retinal nerves for the prevention and/or treatment of corneal pain (e.g., acute corneal pain or chronic corneal pain, including but not limited to paraocular pain, extraocular pain, and post-herpetic neuralgia), glaucoma, genetic retinal diseases or disorders and genetic-based retinopathies (e.g., diabetic retinopathy). Other beneficial uses of the compositions and methods of the invention in treating, preventing, ameliorating or diagnosing nerve and nervous system diseases or disorders will be familiar to the ordinarily skilled artisan based on the guidance provided herein in view of information readily available in the relevant arts.

According to this aspect of the invention, methods of treating or preventing a nerve or nervous system disease or disorder in a human or veterinary animal suffering from or predisposed to a nerve or nervous system disease or disorder comprise administering the compositions described herein, particularly the CMPs and CMP-TC conjugates and compositions comprising such CMPs and/or conjugates, into or near one or more tissues, organs or organ systems of a human or veterinary animal, at a site proximal to the location of a nerve or nervous system lesion associated with or causing the nerve or nervous system disease or disorder. Without wishing to be bound by theory, the inventors surmise that in areas of certain nerve or nervous system diseases and disorders there is sufficient disruption of type I collagen (perhaps among other components of the local extracellular matrix) such that the CMP will target the site of the nerve or nervous system disease or disorder specifically and intercalate into the collagen structure, thereby inducing neuroregeneration and/or neuroprotection directly via reformation of a functioning collagen matrix, or in cases where the CMP carries a therapeutic compound delivering the therapeutic compound to the site where it must act to treat, prevent or ameliorate the nerve or nervous system disease or disorder. According to this aspect of the invention, the conjugates or compositions are suitably applied to, near or into the tissue, organ or organ system in a dosage sufficient to treat, prevent or ameliorate the nerve or nervous system disease or disorder, and the progression, remission or stasis of the nerve or nervous system disease or disorder in the human or veterinary animal is then monitored over time for improvement in the disease or disorder state. Additional concentrations and amounts of the conjugates or compositions of the invention that are suitably used in such methods can be easily determined by one of ordinary skill, based on the information contained herein and that is available in the art, without the need to resort to undue experimentation. If necessary, the conjugate or composition of the invention is then periodically readministered into, near or onto one or more tissues, organs or organ systems according to dosing and treatment schedules and protocols described herein and that will be familiar to the ordinarily skilled artisan, until the nerve or nervous system disease or disorder is cured, prevented or ameliorated. In such embodiments, the conjugates or compositions of the invention are suitably administered to, near, on or into the tissues, organs or organ systems parenterally or topically. Parenteral administration is accomplished by any art-known route of administration of a therapy to the tissues, organ or organ systems, for example via a route selected from the group consisting of subcutaneous injection, intradermal injection, intramuscular injection, intracranial injection, intraspinal injection, or injection into any tissue, organ or organ system where a nerve or nervous system disease or disorder is being manifested; intravenous infusion; intraarterial infusion; endoscopic application; transdermal diffusion; implantation of a drug eluting wafer, film, gel or putty; sublingually; orally; or rectally. In certain such methods, the composition is suitably administered parenterally to the human or veterinary animal in the form of an injected solution or paste, a pill, a capsule, a solution, a suspension or a powder that is inhaled or ingested by the human or veterinary animal, or in the form of a mesh, film, wafer, gel, sphere, nanoparticle, paste, putty or patch that is implanted near, on or into the tissue, organ or organ system at or proximal to the site of the nerve or nervous system disease or disorder. In certain such embodiments, one or more of the compounds, compositions or conjugates of the invention may be coated onto or into a mesh or “sleeve” material such that the mesh or sleeve material is impregnated with one or more of the compounds, compositions or compositions of the invention, and the mesh or sleeve then applied to an injured (e.g., transected) or damaged nerve, nerve process or nerve bundle.

In other embodiments, administration of the conjugates or compositions to, near or into the tissues, organs or organ systems can be accomplished by any well-known means, including in the form of a solution, an ointment, a salve, a patch, a film, a gel, a paste, spheres, nanoparticles, putty, a cream, a topical solution and a drug eluting wafer. For example, the conjugates or compositions can be applied to or near, or introduced into, the tissues, organs or organ systems in the form of one or more drops of solution or a suspension that contains the composition or conjugates (for example, for use in the back of the eye, in the form of a topical transocular eyedrop); via injection; in the form of a coating on a solid material that is implanted into, near or onto the tissues, organs or organ systems; in the form of a mesh or patch; by attaching the conjugate or composition to, or enclosing it within, one or more nanoparticles that are then delivered into, near or on the tissues, organs or organ systems. Other suitable methods of applying the conjugates or compositions to, on, near or into the tissues, organs or organ systems to accomplish the therapeutic and diagnostic methods of the invention will be readily apparent to the ordinarily skilled artisan.

In related embodiments, the invention provides devices, particularly medical devices, suitable for treating or preventing a disease, disorder or medical condition in a human or veterinary animal suffering from or predisposed to said disease, disorder or medical condition. Such devices suitably will comprise at least one of the compositions of the present invention, in the form of a coating on the device or a composition that is embedded within the device such that it is released from or elutes from the device once implanted within the body of the human or veterinary animal. Suitable such devices include, but are not limited to, artificial joints, stents, catheters, sutures, bone screws, bone plates, prosthetics (e.g., artificial limbs, body structures, organs, etc.), absorbable or non-absorbable meshes, absorbable or non-absorbable patches, drug-releasing wafers, brain neurostimulators (e.g., deep brain neurostimulators), gastric stimulators, cochlear implants, cardiac defibrillators, cardiac pacemakers, insulin pumps, internal infusion pumps, and the like. Suitable other devices useful in accordance with this aspect of the invention will be readily apparent to the ordinarily skilled artisan.

The devices provided by this aspect of the invention are useful for treating, preventing, ameliorating or diagnosing diseases, disorders and medical conditions in humans or veterinary animals suffering from or predisposed to such diseases, disorders or medical conditions. In methods according to this aspect, one or more medical devices of the invention is implanted into the human or veterinary animal, and medical condition of the human or veterinary animal is monitored until the disease, disorder or medical condition is cured, ameliorated or prevented in the human or veterinary animal. Suitable diseases, disorders and medical conditions that may be cured, treated, ameliorated or prevented using the devices and methods of the invention include cancers (such as those described elsewhere herein), and diseases or disorders affecting an organ system of the human or veterinary animal including the integumentary system (particularly diseases or disorders of the skin such as those described in detail herein), the muscular system, the skeletal system (particularly diseases or disorder of the bones, joints, cartilage, tendons or ligaments such as those described in detail herein), the nervous system (particularly those of the brain or the eye (such as anterior segment eye diseases and disorders including but not limited to those involving the corneal nerves (such as corneal pain (which may be acute or chronic), including but not limited to that resulting from damage or injury to the corneal nerves or denervation, e.g., paraocular pain, extraocular pain, and post-herpetic neuralgia), glaucoma, cataracts, vitreous adhesions or floaters, macular degeneration, dry eye syndrome, corneal keratitis, non-infectious corneal ulceration, non-infectious corneal melting, infectious corneal ulceration, infectious corneal melting, conjunctivitis, Stevens-Johnson Syndrome, scleritis, episcleritis, iritis, uveitis, vitritis, Behcet disease uveitis, birdshot retinochoroidopathy, juvenile idiopathic arthritis (JIA)-associated uveitis, multifocal choroiditis with panuveitis, necrotizing scleritis, serpiginous choroidopathy, sympathetic ophthalmia, Vogt-Koyanagi-Harada (VKH) disease, non-infectious panuveitis, ectasia, keratoconus, corneal laceration, corneal erosion, corneal abrasion, and a post-operative affliction of the eye resulting from eye surgery such as a post-operative cataract surgery state requiring medication or a post-operative glaucoma surgery state requiring medication, or posterior segment eye disorders such as those involving the retina, retinal epithelium (particularly the retinal pigment epithelium), retinal blood vessels, retinal nerves or optic nerve, including but not limited to macular degeneration (wet, dry and age-related), retinitis pigmentosa, retinal tears and detachment, retinopathy (e.g., diabetic retinopathy), retinal arterial or venous occlusion (e.g., BRAO (Branch Retinal Artery Occlusion), CRAO (Central Retinal Artery Occlusion), BRVO (Branch Retinal Vein Occlusion) and CRVO (Central Retinal Vein Occlusion), optic neuritis, optic neuropathy (including, for example, AION (Anterior Ischemic Optic Neuropathy), traumatic optic neuropathy and optic atrophy (e.g., glaucomatous optic atrophy)), and other neuropathies impacting the eye or area around the eye, including paraocular diseases, disorders and medical conditions and extraocular diseases, disorders and medical conditions, such as cranial nerve palsies including but not limited to Cranial III Nerve Palsy, Cranial Nerve IV Palsy, Cranial Nerve V Palsy (e.g., trigeminal neuralgia and post-herpes zoster neuralgia), Cranial Nerve VI Palsy and Cranial Nerve VII Palsy (e.g., Bell's Palsy), the circulatory system, the lymphatic system, the respiratory system (including those diseases or disorders affecting the epiglottis, the trachea, a bronchus, a bronchiole or a lung in the human or veterinary animal, particularly those diseases and disorders described in detail herein), the endocrine system, the urinary/excretory system (including those diseases or disorders affecting the kidney, the ureter, the urinary bladder, the upper urinary tract (i.e., the renal pelvis), the ureter or the urethra of the human or veterinary animal, particularly those diseases and disorders described in detail herein), the reproductive system (including diseases and disorders affecting the testicle, the prostate, the penis, the vagina, the cervix, the uterus, a fallopian tube or an ovary in said human or veterinary animal, particularly those diseases and disorders described in detail herein), the digestive system (including those diseases or disorders affecting the esophagus, stomach, small intestine, colon or rectum in said human or veterinary animal, particularly those diseases and disorders described in detail herein), and nerves or the nervous system (including the peripheral nervous system and the central nervous system, particularly those nerve or nervous system disorders, diseases and injuries described in detail herein). Suitable methods for implanting one or more of the devices provided by this aspect of the invention into a human or veterinary animal, to accomplish the treatment, prevention, amelioration or diagnosis of a disease, disorder or medical or physical condition in the human or veterinary animal will be familiar to the person of ordinary skill in the relevant medical and surgical arts.

Concentrations of the CMPs, or of the CMP-TC conjugates, useful in treating, preventing, ameliorating or diagnosing one or more diseases or disorders according to the methods of the present invention will be readily apparent to the artisan ordinarily skilled in the pharmaceutical and medical arts. For unconjugated CMPs, suitable amounts or concentrations of CMPs to be administered to a subject, particularly a human or veterinary animal, suitable amounts or concentrations of CMPs to be used include those described hereinabove. Based on the guidance provided herein, one of ordinary skill in the medical, pharmaceutical and/or pharmacological arts can determine the appropriate amount of the conjugates and compositions of the invention to be used per kilogram (kg) of body mass of the human or veterinary animal. For conjugated CMP-TCs, the same amounts or concentrations of CMPs described herein, whether in concentration (e.g., ng/ml or μg/ml) or in amount (e.g., mg per kg of body mass), are suitably administered to the subject, and the amount of active pharmaceutical ingredient or biologic is calculated during the conjugation process to deliver therapeutically effective amounts of the desired active pharmaceutical ingredient or biologic, depending upon the disease or disorder that is to be treated, prevented, ameliorated or diagnosed in the human or veterinary animal. Suitable amounts or concentrations of active pharmaceutical ingredients or biologics to be used according to this aspect of the invention will be familiar to the ordinarily skilled artisan, and can be readily determined from information contained herein and other information that is available in the relevant arts.

It will be readily apparent to one of ordinary skill in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1: Effect of CMPs and CMP-TC Conjugates in Treating Myopia

To examine the possible therapeutic effects of CMPs of the invention in treating myopia, studies were designed to test certain CMPs conjugates in an in vivo setting—the treatment and possible reversal of myopia in laboratory animals, such as mice and rats. In such studies, animals are anesthetized, and the eyes are treated dropwise over 1-3 days with matrix 60 nM human metalloproteinase 1 (MMP-1; Biolegend, San Diego, CA) that is pre-activated with 2 mM 4-aminophenylmercuric acetate (APMA, Sigma, St. Louis, MO) as described previously (Ribeiro, M. et al., Int. J. Mol. Sci. 23:7004 (2022)). Negative control animals are treated with vehicle only (PBS). Following MMP treatment, animals are treated dropwise daily for up to 3 weeks with 25 μg/ml or 50 μg/ml CMP (particularly SEQ ID NO:1 or SEQ ID NO:6) or with vehicle (PBS) for negative control animals. Following these treatments, the eye axial length is measured using standard optometric techniques, and the structure of the scleral collagen matrix is determined using a variety of microscopic techniques including confocal microscopy, second harmonic generation microscopy (Guo, P. et al., Trans. Vis. Sci. Tech. 9(9):45 (2020)), and the like. Results of these studies, correlating increased collagen disaggregation and disorganization with increased ocular axial length, demonstrate that the collagen matrix in the sclera is involved in maintaining optimal eye structure and shape, and that disruptions in the collagen matrix are associated with decreased visual acuity and the development of myopia. In addition, the results of these studies demonstrate that animals treated with CMP-containing compositions of the invention show an increased level of collagen organization, and restoration of the underlying collagen matrix structure in the sclera, resulting in a reduction in ocular axial length which can be used to indicate treatment of the symptoms and/or signs of myopia. Thus, the CMPs and CMP-TC conjugates of the present invention, and compositions comprising them, are useful in promoting a reduction in axial length of animal eyes in vivo, as a model of a variety of human and veterinary animal ocular conditions including myopia and presbyopia.

### Example 2: Activity of CMPs in Restoring Collagen Matrix Structure and Function in Peripapillary Sclera in Mouse Eyes

The sclera of the eye in mammals is enriched in collagen, which with elastin makes up the majority of the sclera (Guo, P. et al., Trans. Vis. Sci. Tech. 9:45 (2020)). Collagen in the sclera appears to mediate scleral stiffness, and thus the shape of the eye in humans and veterinary animals (Schultz, D. S. et al., Invest. Ophthalmol. Vis. Sci. 49:4232-4236 (2008)). A number of studies have shown that changes in collagen structure, including a thinning of collagen bundles and a reduction in the size of the individual collagen bundles in the corneoscleral, can lead to a reduction in the stiffness of the sclera. This reduction in scleral stiffness in turn increases the permeability of the corneoscleral as well as affecting the shape of the eye, leading to axial elongation of the globe and the development of myopia (Jonas, J. B. and Xu, I., Eye 28:113-117 (2014); Guo, P. et al., Trans. Vis. Sci. Tech. 9:45 (2020); Hou, W. et al., Eye Contact Lens 44:248-259 (2018); Xue, C. C., et al., Br. J. Ophthalmol., doi:10.1136/bjophthalmol-2022-321868 (2022); Pugazhendhi, S., et al., Clin. Ophthalmol. 14:853-873 (2020); Metlapally, R. and Wildsoet, C. F., Prog. Mol. Biol. Transl. Sci 134: 241-248 (2015)). Some studies have found that increased expression of matrix metalloproteinases (MMPs) in the scleral matrix and concomitant disruption of the scleral collagen matrix, which may arise via genetic, environmental or inflammatory etiologies, can lead to the development of myopia in mice (Zhao, F. et al., Am. J. Pathol. 188: 1754-1767 (2018); Guo, P. et al., Trans. Vis. Sci. Tech. 9:45 (2020)). Such effects on the corneoscleral matrix may similarly lead to other ocular diseases and disorders such as glaucoma, cataracts, staphyloma and retinal complications (Wang, B., et al., PLoS ONE 12:e0175913, https://doi.org/10.1371/journal.pone.0175913 (2017)).

Efforts to treat or prevent myopia by strengthening the sclera have met with varying degrees of success. For example, treatment of myopic eyes with atropine can slow the progression of myopia (Upadhyay, A. and Beuerman, R. W., Eye & Contact Lens 46:129-135 (2020); Pugazhendhi, S., et al., Clin. Ophthalmol. 14:853-873 (2020); Gwiazda, J., Optom. Vis. Sci. 86:624-628 (2009)), apparently by inducing the production of type I collagen in scleral fibroblasts (Cristaldi, M. et al., Biomedicines 8:78 (2020)). In other studies, collagen fibrils in the corneoscleral matrix have been stiffened via cross-linking, either chemically or via photoinduction (Guo, P. et al., Trans. Vis. Sci. Tech. 9:45 (2020); Backhouse, S. and Gentle, A., Ann. Eye Sci. 3:5 (2018); Garcia, M. B. et al., Invest. Ophthalmol. Vis. Sci. 58:1875-1886 (2017); Elsheikh, A., and Phillipa, J. R., Ophthalmic Physiol. Opt. 33:385-389 (2013); Wang, W.-Y. et al., Biomed. Pharmacother. 133:111092 (2021)). One downside of such approaches to treating or preventing myopia, however, is that artificial connective tissue remodeling such as those described in the literature cited above may increase the risk of glaucoma in patients receiving such treatments (Grytz, R., et al., Curr. Opin. Biomed. Eng. 15:40-50 (2020)).

The present inventors therefore reasoned that a method of stiffening the corneoscleral matrix that would more closely resemble natural collagen repair, such as occurs during treatment of mammalian eyes with formulations comprising one or more collagen mimetic peptides (CMPs) including those of the present invention, could be useful in treating myopia and preventing its progression by restoring the structure and function of the collagen matrix in the corneosclera. Such approaches could therefore be useful in treating or preventing a variety of corneoscleral diseases and disorders, including myopia, presbyopia, scleritis, episcleritis and keratoconus, among others.

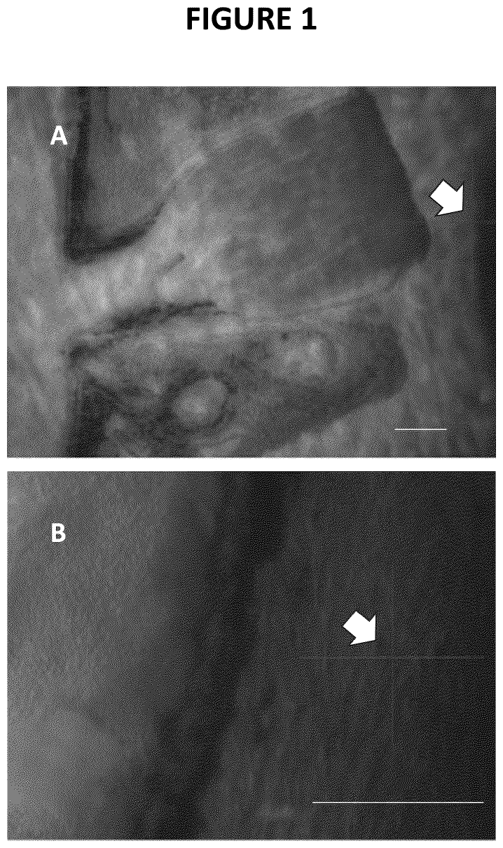

Therefore, to examine the possible therapeutic effects of CMPs of the invention in treating myopia, studies were designed to test the effects of certain CMPs conjugates in a model of the in vivo setting—an ex vivo study of the collagen matrix in the peripapillary sclera in mouse eyes. To conduct these studies, mouse eyes were explanted and thin sectioned, and sections of the peripapillary sclera (the scleral area adjacent to the optic nerve head) were examined by atomic force microscopy (AFM). FIG. 1 shows the morphology of the peripapillary sclera in a representative sample of such sections, with the arrows in FIG. 1A (lower-power view) and FIG. 1B (higher-power view of the same section) showing the area targeted by the AFM probe that is able to measure Young's Modulus in that tissue area as a determinant of the structural stiffness or elasticity of the point being measured (see, e.g., Girard, M. J. et al., J. Biomech. Eng. 131:051012, doi:10.1115/1.3113683 (2009)). Specifically, measurement of a higher Young's Modulus at a given point via AFM indicated that the sclera was less elastic or stiffer and more resistant to deformation than an area at which a lower Young's Modulus was measured.

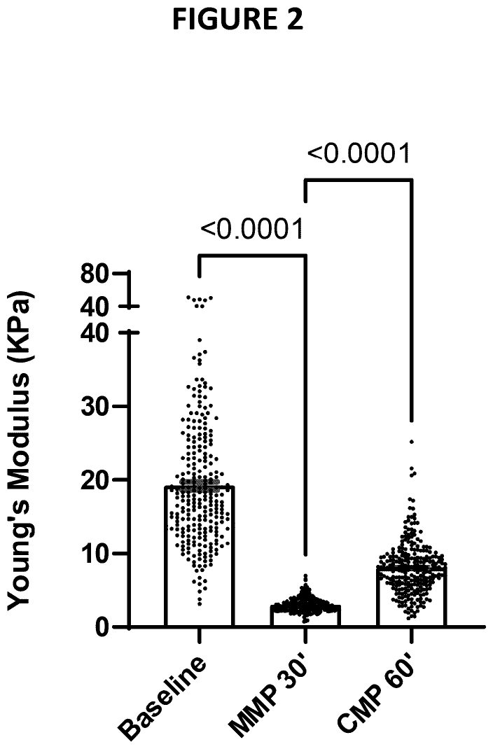

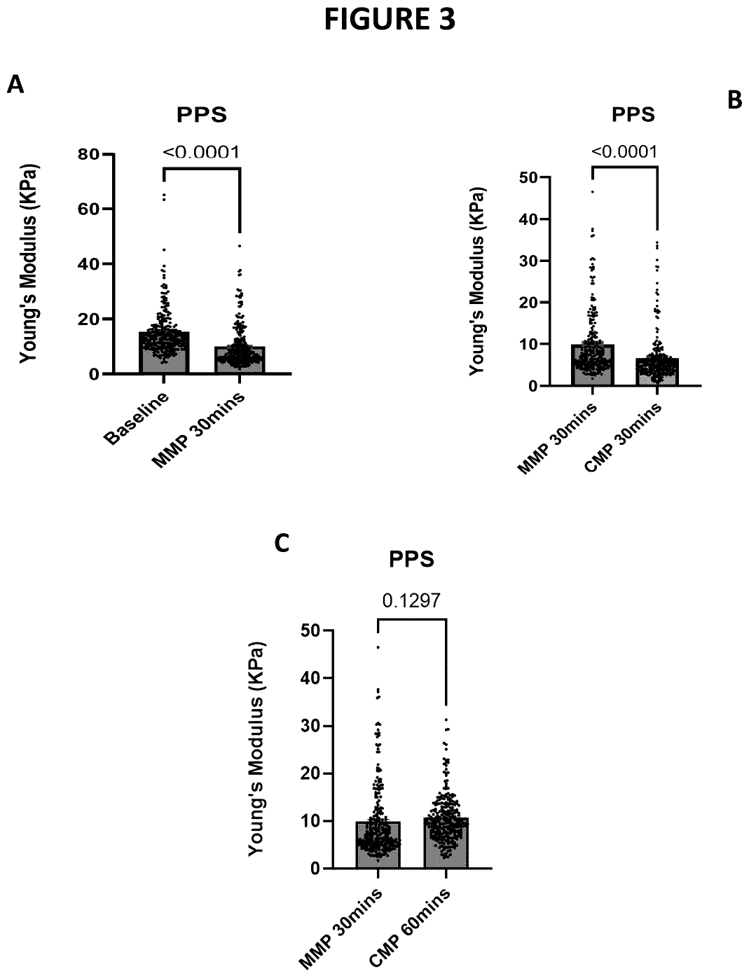

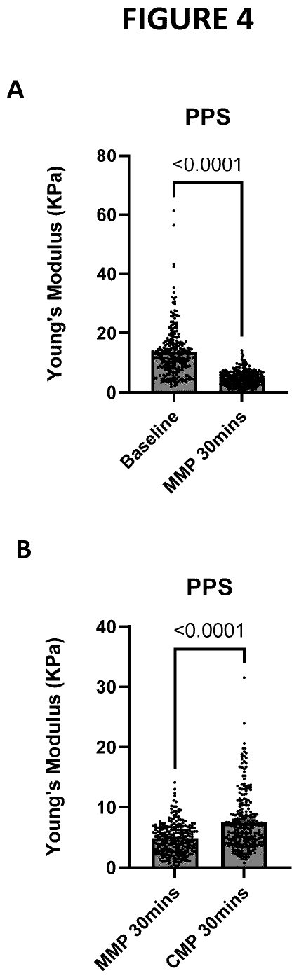

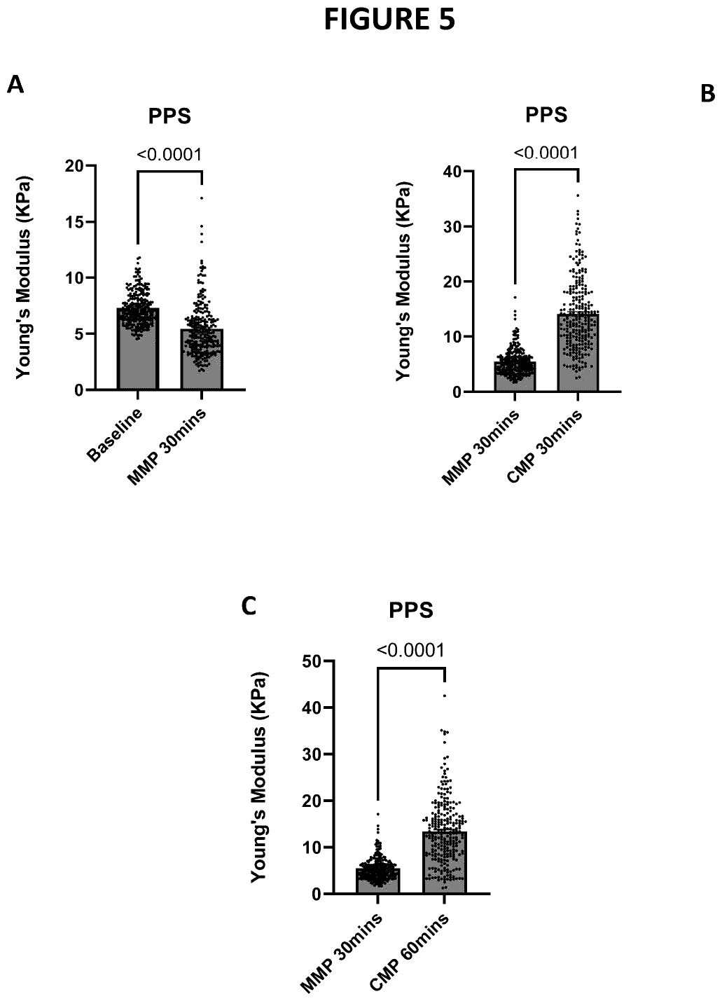

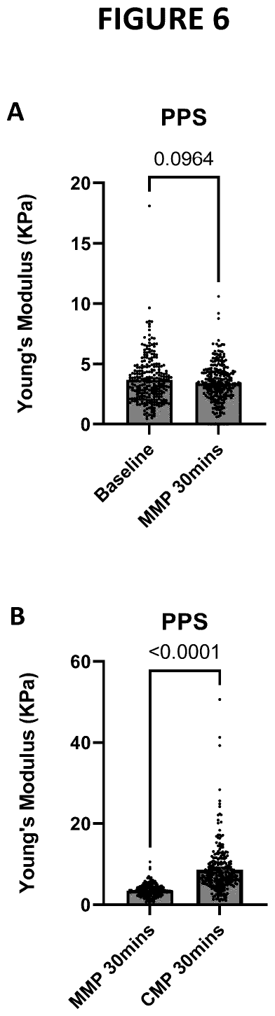

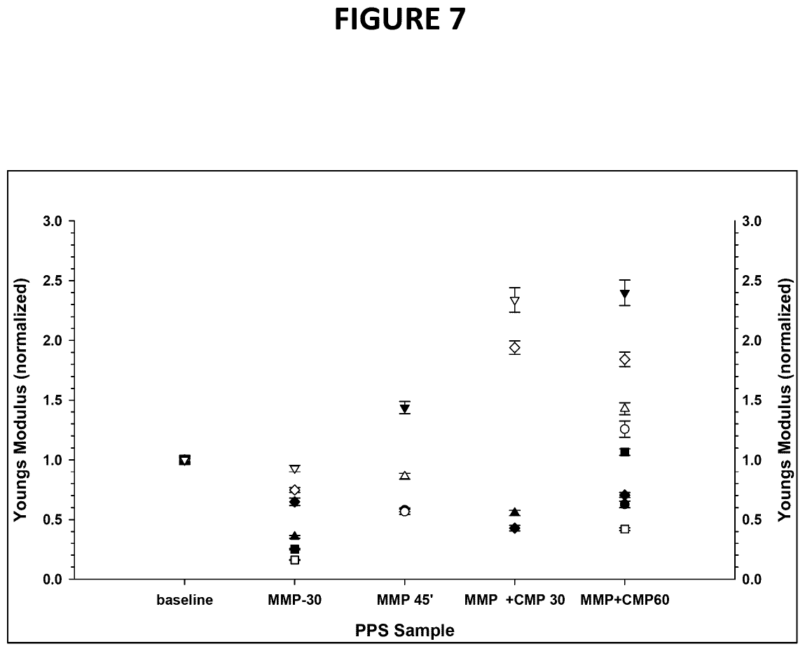

To examine the impact of matrix metalloproteinase (MMP) treatment, with or without subsequent CMP treatment, on the stiffness of the mouse peripapillary scleral explants, samples were treated with MMP (50 μg/ml for 30 minutes), followed by either a PBS washout or by treatment with a CMP-containing formulation of the invention for 60 minutes. AFM was then used to measure the Young's Modulus in a number of different locations, and results are shown in FIG. 2. Baseline (i.e., untreated) explant samples showed a variety of moduli of stiffness across the surface of the sclera, as expected since stiffness of the sclera varies from point to point in normal and myopic eyes (Grytz, R., et al., Curr. Opin. Biomed. Eng. 15:40-50 (2020); Jonas, J. B. and Xu, I., Eye 28:113-117 (2014)). Treatment of sclderal explants with MMP significantly reduced the scleral stiffness (represented by a decrease in Young's Modulus) nearly uniformly, and substantially below baseline; this result is consistent with what is seen in advanced myopia in humans and other animals. In contrast, treatment with a CMP3 (SEQ ID NO:1)-containing formulation of the invention for 60 minutes increased the scleral stiffening significantly above what was seen with MMP-treated samples, indicating that the CMP treatment was able to at least partially reverse the disruption of the scleral matrix caused by the MMP. Similar results were observed upon treatment of MMP-treated samples with CMP3-containing formulations for 30 minutes (FIGS. 3A-3B, 4A-4B, 5A-5B, 6A-6B), although in some samples treatment with CMP for 60 minutes (FIGS. 3B-3C, 5B-5C) provided more enhanced restoration of the scleral stiffness. When these results were plotted in a scatter graph (Figure. 7), it was apparent that treatment of MMP-treated scleral explants with CMP-containing formulations for as little as 30 minutes induced a significant stiffening in the scleral matrix, while increasing the treatment with CMPs for 60 minutes appeared to enhance the response to some extent beyond what was seen with 30-minute treatment.

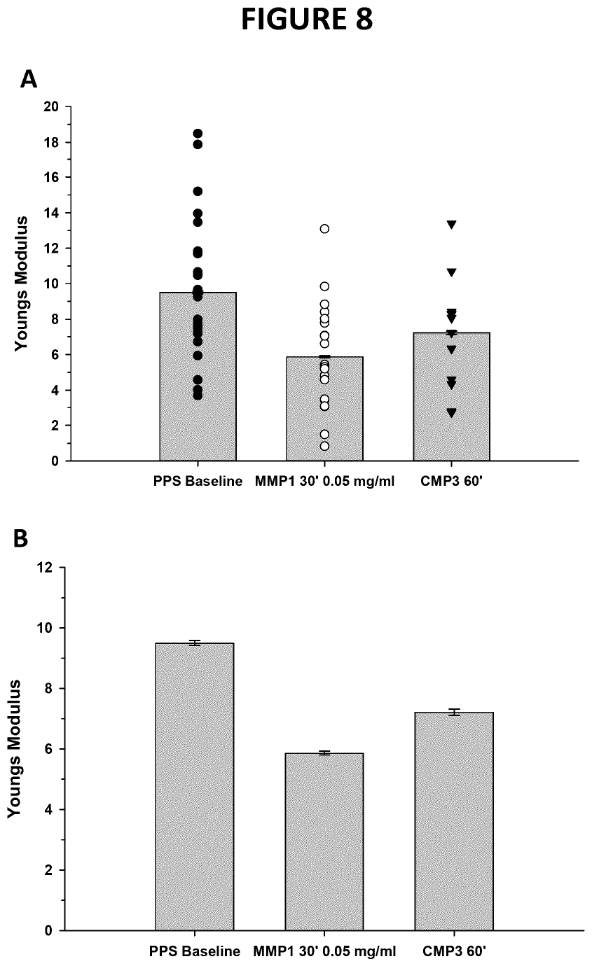

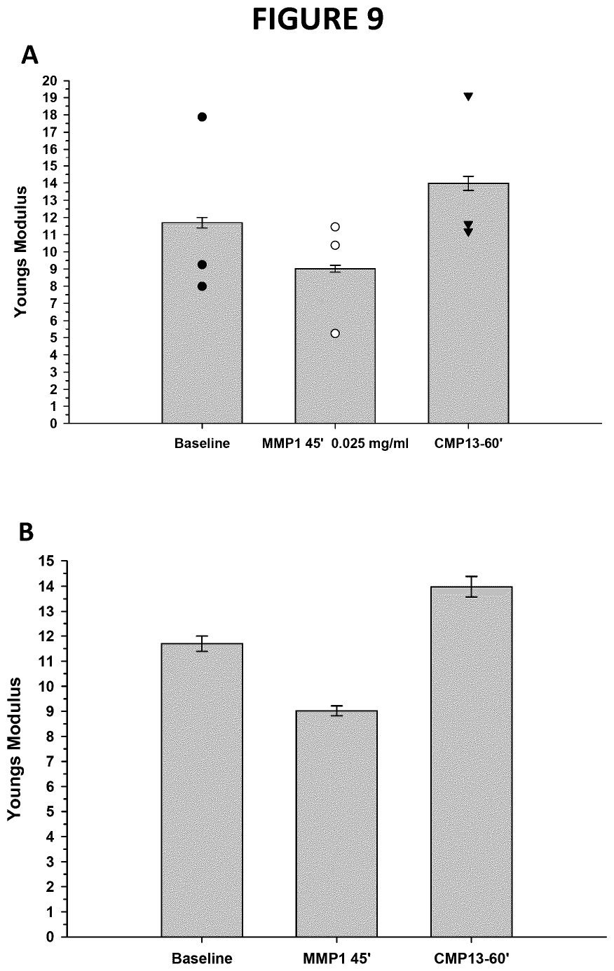

To increase the statistical significance of these studies, AFM was used to make Young's Modulus measurements across a large number of points in multiple individual locations in a given sample of peripapillary sclera. As was done above, samples were left untreated (baseline), or were treated with 50 μg/ml MMP1 for 30 minutes and then with a CMP-containing formulation of the invention (either CMP3, i.e., SEQ ID NO:1 or CMP13, i.e., SEQ ID NO:6) for 60 minutes, and then Young's Modulus was measured at multiple points in multiple locations via AFM. Results are shown in FIGS. 8 and 9.

As seen in FIG. 8, and as seen in the preceding figures, treatment of the scleral explants with MMP1 resulted in a significant decrease in Young's Modulus in the samples compared to untreated (baseline) samples. Treatment with CMP3-containing formulations, however, rapidly increased the scleral stiffness (FIG. 8A), with very tight statistical significance (ANOVA p<0.001) between the groups (FIG. 8B). Analogous results were observed with treatment of the peripapillary scleral samples with a CMP13-containing formulation of the invention (FIGS. 9A, 9B). Indeed, it appeared that treatment with CMP13 (SEQ ID NO:6) enhanced the stiffness of the peripapillary sclera beyond that observed in the untreated baseline samples.

Taken together, these results demonstrate that the CMP-containing formulations of the present invention can reverse the collagen matrix disruption, and thus restore (or at least partially restore) the strength and stiffness of the peripapillary sclera in mouse eye explants. Since CMPs are, by definition, mimetics of naturally occurring collagen strands, these results indicate that the CMP-containing formulations of the present invention, and methods using such formulations, may be useful in treating ocular diseases and disorders characterized by a disruption of the scleral structure and stiffness with fewer side effects than what is seen in more traditional treatments such as atropine or collagen cross-linking.

### Example 3: Activity of CMPs in Restoring Collagen Matrix Structure and Function in the Glial Lamina in Mouse Eyes

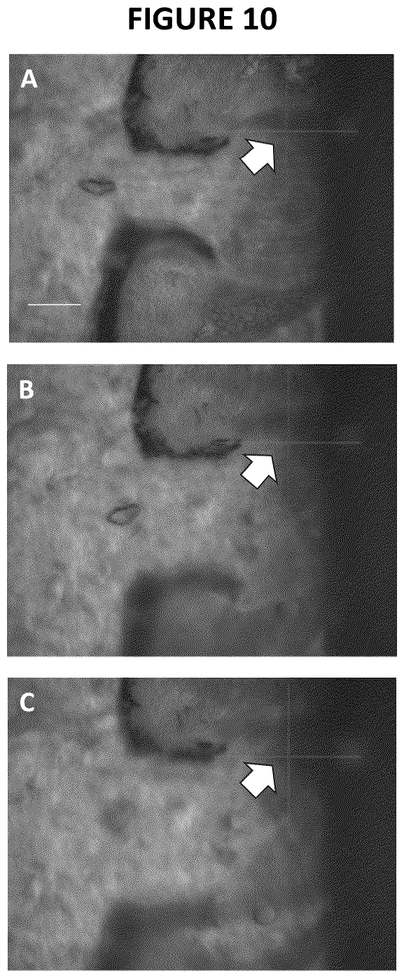

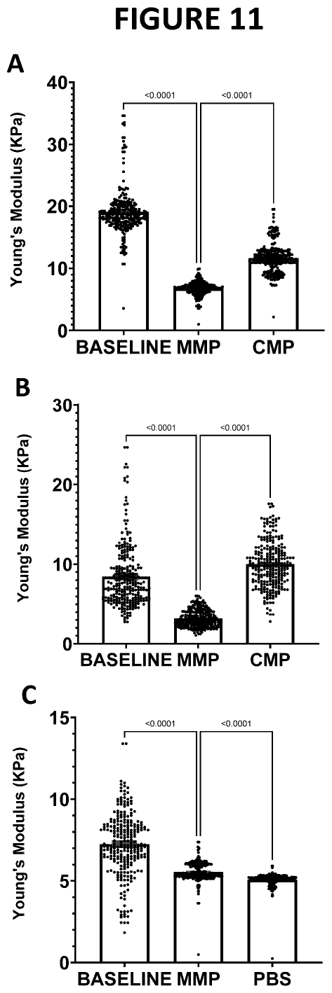

Building upon the studies detailed in Example 2 herein, the present inventors reasoned that since CMPs appear to be useful in reforming the collagen fibril bundle structures in the mouse peripapillary sclera and thereby strengthening the scleral matrix, such CMP-containing formulations may be similarly useful in strengthening the collagen structures in areas adjacent to the peripapillary sclera—notably, the glial lamina at the optic nerve head. To conduct these studies, mouse eyes were explanted and thin sectioned as described in Example 2 above, and sections of the glial lamina and the peripapillary sclera were examined by atomic force microscopy (AFM). FIG. 10 shows the morphology of the peripapillary sclera and glial lamina in three representative samples of such sections, with the arrows showing the area targeted by the AFM probe that is able to measure Young's Modulus in that tissue area. FIG. 10A is a sample that was untreated, while FIG. 10B is a sample after treatment for 30 minutes with 5 μg/ml MMP1 and FIG. 10C is a sample after treatment for 30 minutes with 5 μg/ml MMP1 followed by treatment for 30 minutes with a CMP formulation of the invention (comprising SEQ ID NO:6, or “CMP13”). As can be readily observed in these photomicrographs, there did not appear to be an observable difference in microscopic morphology between these samples. However, as seen in FIG. 11, measurement of Young's Modulus in the glial lamina between these three samples demonstrated substantial differences in the tissue stiffness, with MMP adversely affecting the stiffness of the glial lamina in a way reminiscent of what was seen with the peripapillary sclera samples (FIGS. 11A-11C) In contrast, the CMP-containing formulation of the invention restored the rigidity of the tissue closer to (FIG. 11A) and in some cases even surpassing (FIG. 111B) what was observed for baseline untreated samples. To ensure that this effect was not simply due to washout of the MMP upon treatment with CMP-containing formulations, MMP-treated samples were treated with vehicle (PBS); such treatment did not restore the stiffness of the glial lamina which approximated that seen in MMP-treated samples (FIG. 11C).

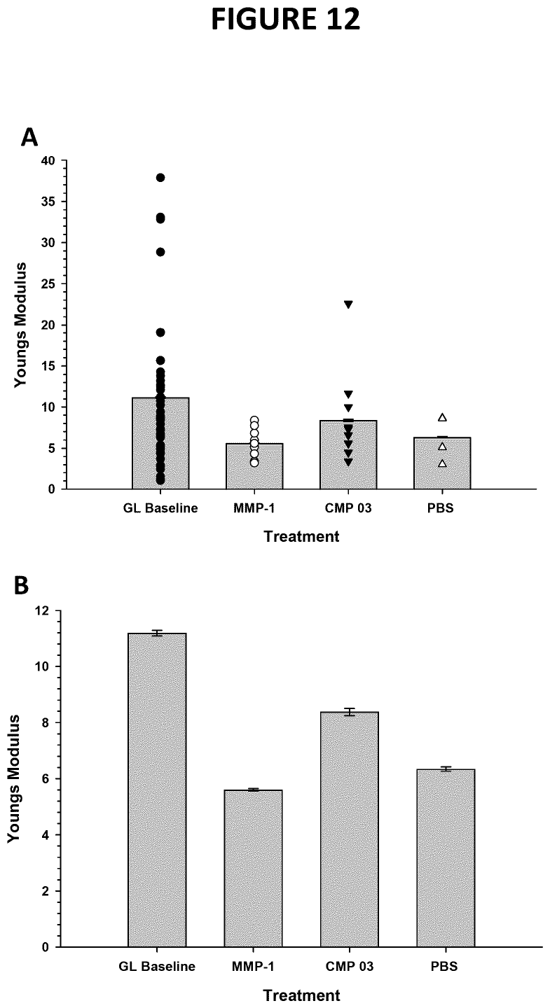

To increase the statistical significance of these studies, AFM was used to make Young's Modulus measurements across a large number of points in multiple individual locations in a given sample of glial lamina, as was done in Example 2 for peripapillary sclera. Samples were left untreated (baseline), or were treated with 5 μg/ml MMP1 for 30 minutes and then with a CMP-containing formulation of the invention (CMP3, i.e., SEQ ID NO:1) for 60 minutes, and then Young's Modulus was measured at multiple points in multiple locations via AFM. Results are shown in FIG. 12. As was observed for peripapillary sclera and as seen with a lower number of glial lamina samples in FIG. 11, treatment of the glial lamina with MMP1 resulted in a significant decrease in Young's Modulus in the samples compared to untreated (baseline) samples. Treatment with CMP3-containing formulations, however, rapidly increased the glial lamina stiffness (FIG. 12A), with very tight statistical significance (ANOVA p<0.001) between the groups (FIG. 12B).

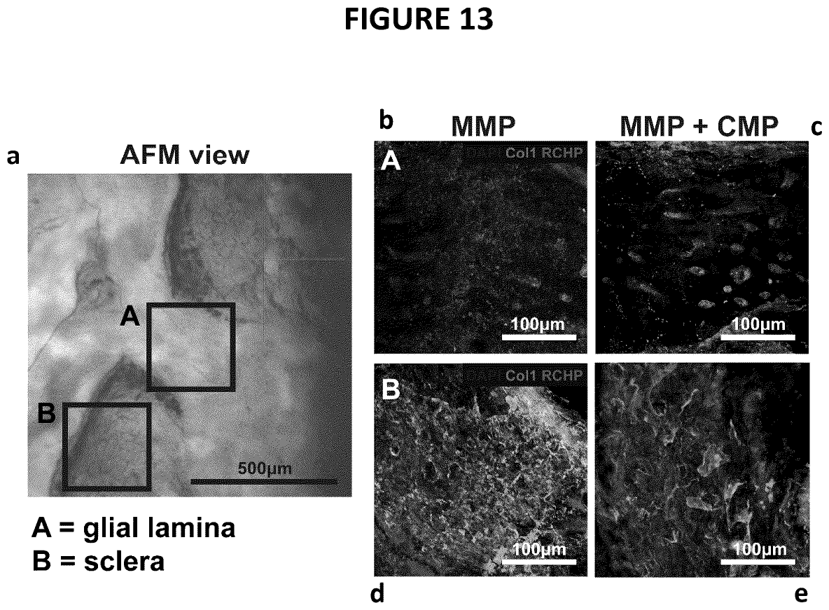

To examine the distribution of disrupted and intact collagen in such explants, samples prepared as above were treated with DAPI (to localize cell nuclei), a fluorescently labeled antibody against type I collagen (“Col1”), and a Cy3-labeled collagen hybridizing peptide (“RCHP”) which binds only to areas of disrupted collagen. Results are shown in FIG. 13. AFM examination of the morphology of these samples was reminiscent of those seen above and in Example 2 (FIG. 13a) and allowed the discernment of the glial lamina (“A”) and the peripapillary sclera (“B”) in such samples. Fluorescence microscopy for the various labels indicated that there was a large amount of disrupted collagen in MMP-treated samples in both the glial lamina (FIG. 13b) and peripapillary sclera (FIG. 13d), and the glial lamina section actually was nearly devoid of intact collagen (FIG. 13b). In contrast, samples treated with CMP3 (i.e., SEQ ID NO:1) showed virtually no disrupted collagen in either the glial lamina (FIG. 13c) or the peripapillary sclera (FIG. 13e), and in fact the glial lamina demonstrated restoration—as quickly as within 60 minutes—of a more robust intact collagen network (FIG. 13c). These results indicate that the CMP-containing formulations of the invention, when used in the methods of the present invention, can rapidly restore the collagen network in mouse eye explants, particularly in the sclera and glial lamina, thus reversing the effects of matrix metalloproteinase degradation of the collagen matrix.

Together with those of Example 2, these results indicate that the CMP-containing formulations of the present invention can reverse the collagen matrix disruption, and thus restore (or at least partially restore) the strength and stiffness of the peripapillary sclera and the glial lamina in mouse eye explants where the structure and function of the collagen matrix have been disrupted. Since such disruption of the collagen matrix is a hallmark of certain corneoscleral diseases and disorders, such as myopia, presbyopia, keratoconus and the like, these formulations and methods of the invention should prove useful in treating ocular diseases and disorders characterized by a disruption of the corneoscleral and optic nerve structure and function with fewer side effects than what is seen in more traditional treatments such as atropine or collagen cross-linking.

### Example 4: Activity of CMPs in Restoring Collagen Structure: Molecular Analysis

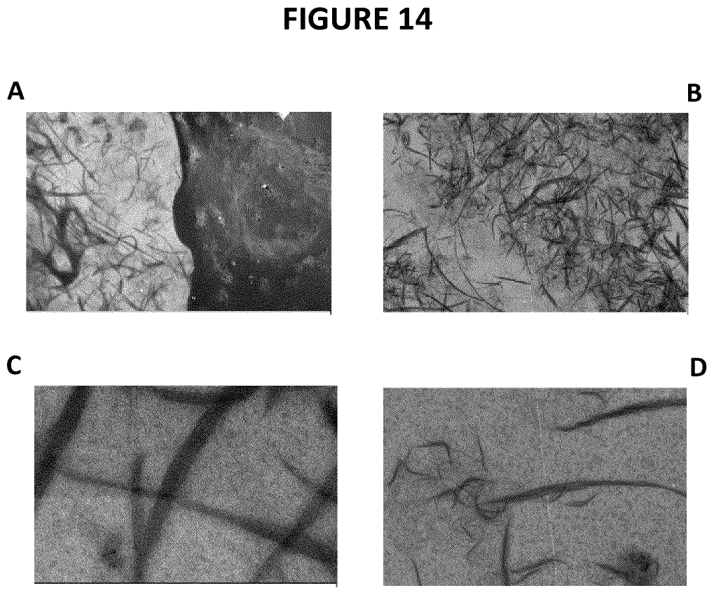

To further examine the impact of the CMP-containing formulations on collagen structure and function at a molecular level, samples of type I collagen plated onto substrates were examined microscopically to examine fiber/fibril formation and the presence or absence of fibril networks. Human type I collagen was plated onto substrates at a concentration of 10 μg/ml in PBS, allowed to air dry, and then left untreated or treated with MMP1 (60 ng/ml) for 30 minutes. Samples were then examined by scanning electron microscopy (SEM); results are shown in FIG. 14. Untreated samples of collagen demonstrated extensive fiber and fibril formation (FIGS. 14A, 14C), with a significant amount of network formation observed at lower power (FIG. 14A) and higher power (FIG. 14C) views. In contrast, MMP treatment of the substrate resulted in a readily observable amount of disruption of the collagen fibers, fibrils and network (FIG. 14B), which at higher magnification (FIG. 14D) was seen to also contain a high amount of collagen fragments and in some cases single strand collagen. Thus, MMP treatment of the collagen substrates resulted in rapid disorganization of the collagen structure and high amounts of disrupted collagen, reminiscent of earlier studies from the same inventors (see, e.g., U.S. Pat. No. 11,389,513, the disclosure of which is incorporated herein by reference).

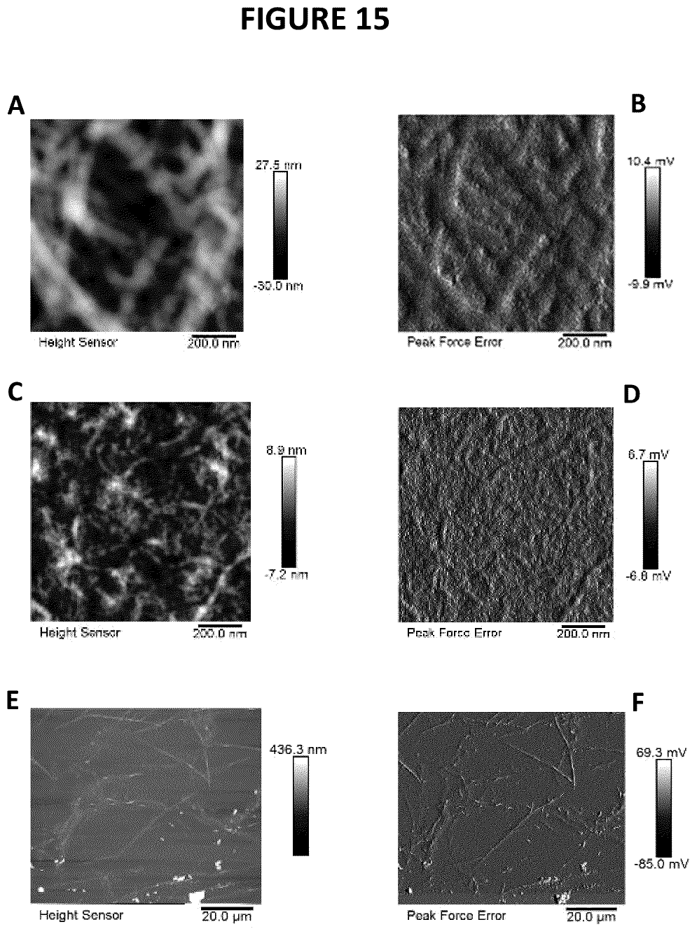

Examination of these same substrates by AFM, but also including a sample that had been treated simultaneously for 30 minutes with 100 ng/ml MMP1 and with 1 μg/ml of a CMP13 (SEQ ID NO:6)-containing formulation of the invention demonstrated the impact of the CMP-containing formulations of the invention in preventing the disruption of the collagen structures seen on the substrates. Results of these studies are shown in FIG. 15. In untreated (baseline) samples, extensive and multilamellar networks of collagen fibers, fibrils and bundles could be readily observed (FIGS. 15A, 15B). MMP-treated samples, however, showed a substantial amount of disorganized collagen and little remaining fiber/fibril formation (FIGS. 15C, 15D). Inclusion of CMP at the same time as the MMP, however, seemed to prevent the impact of the MMP on the collagen networks on the substrate, in that extensive collagen fiber/fibril formation could be observed in such samples (FIG. 15E), along with long-length fiber bundles and networks across the surface of the substrate (FIG. 15F). Together, these studies demonstrate that not only may the CMP-containing formulations of the present invention be useful in treating and reversing the collagen-disrupting effects of MMPs that lead to corneoscleral defects in certain ocular diseases and disorders, but that such formulations may also be useful in preventing such effects. Thus, the CMP-containing formulations and methods of using such formulations provided by the present invention should prove useful in both treating and preventing certain ocular diseases and disorders characterized by a loss of corneoscleral rigidity and strength, including but not limited to myopia, presbyopia and keratoconus.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed. For example, the recitation of a range of values (e.g., a range of dosages or dosing concentrations) should be understood to include the values at the beginning and the end of that range, as well as every value in between those beginning and end values. To illustrate this concept, a range of “about 25 ng/ml to about 250 ng/ml” should be interpreted to include a value that is “about 25 ng/ml,” “about 250 ng/ml,” and every individual concentration value between those two values. The term “about” when used in conjunction with a numeric value typically means a value that is the actual value recited ±10% of that value.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Hence, in addition to those specifically described herein, other suitable embodiments of the invention will be readily apparent to one of ordinary skill in the art based upon the foregoing description and examples, and upon knowledge generally available in the relevant arts. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All references cited herein, including U.S. patents and published patent applications, international patents and patent applications, and journal references or other publicly available documents, are incorporated herein by reference in their entireties to the same extent as if each reference had been specifically cited for the portion or portions of such reference applicable to the section of this application to which it is relevant.

What is claimed is:

1. A composition suitable for use in a medicament for treating or preventing a corneoscleral disease, disorder or condition in a human or veterinary animal in need of treatment or prevention thereof, said composition comprising (a) at least one collagen mimetic peptide (CMP) having, and (b) one or more pharmaceutically suitable carriers.

2. The composition of claim 1, wherein said corneoscleral disease, disorder or condition is selected from the group consisting of keratitis, episcleritis, scleritis, corneal ulceration, sequelae of corneal ulceration, corneal ectasia, acquired abnormalities of corneal shape, keratoconus, corneal astigmatism, keratoglobus, posterior corneal depressions, keratectasia, keratocele, descemetocele, pellucid marginal degeneration, Terrien's marginal dystrophy, Mooren's ulcers, central corneal ulcers, marginal corneal ulcers, staph marginal ulceration, Salzman's nodular dystrophy, age-related peripheral corneal atrophy, geographic ulceration, disciform stromal keratitis, metaherpetic ulceration, keratomalacia, post penetrating keratoplasty, incisional wounds, anterior membrane dystrophies, stromal dystrophies, ocular mucous membrane pemphigoid, necrotizing scleritis, scleromalacia, coloboma, myopia, presbyopia, hyperopia, scleral buckle induced scleromalacia, congenital hereditary stromal dystrophy, congenital anterior staphyloma, sclerocornea, traumatic breaks in Descemet's membrane, corneal keloids, scleral ectasia, scleral staphyloma, deep scleritis, necrotizing scleritis, scleromalacia perforans, hyaline degeneration of the sclera, paralimbal scleromalacia, ocular graft vs host disease, and choroideremia.

3. The composition of claim 1, wherein said corneoscleral disease, disorder or condition is myopia, presbyopia or keratoconus.

4. The composition of claim 1, wherein said at least one CMP is attached to at least one therapeutic compound (TC) to form a CMP-TC conjugate.

5. The composition of claim 1, wherein said at least one collagen mimetic peptide has an amino acid sequence corresponding to any one of SEQ ID NOs:1-388, 397-416, and 418-470.

6. The composition of claim 1, wherein said at least one collagen mimetic peptide has an amino acid sequence corresponding to any one of SEQ ID NOs: 1-14, 66-94, 107-135, 136-140, 192-220, 233-261, 260-264, 280, 281, 293, 294, 306, 307, 318-346, 347, 348, 359-388, 397-416 and 418-452.

7. The composition of claim 1, wherein said at least one collagen mimetic peptide has an amino acid sequence corresponding to SEQ ID NO:1.

8. The composition of claim 1, wherein said at least one collagen mimetic peptide has an amino acid sequence corresponding to SEQ ID NO:6.

9. The composition of claim 1, wherein said at least one collagen mimetic peptide has an amino acid sequence corresponding to any one of SEQ ID NOs:4, 5 and 9.

10. The composition of claim 1, wherein said at least one collagen mimetic peptide has an amino acid sequence corresponding to any one of SEQ ID NOs: 388, 397-416 and 418-470.

11. The composition of claim 1, wherein said at least one collagen mimetic peptide has an amino acid sequence corresponding to any one of SEQ ID NOs:10-27, 81-94, 122-135, 207-220, 248-261, 333-346, and 374-387.

12. A method of treating or preventing a corneoscleral disease, disorder or condition in a human or veterinary animal in need of treatment or prevention thereof, comprising administering the composition of claim 5 to an eye of said human or veterinary animal in a dosage sufficient to treat or prevent said myopia, keratoconus or presbyopia, monitoring the condition of the eye in said human or veterinary animal over time, and readministering said composition to the eye until said corneoscleral disease, disorder or condition is cured, prevented or ameliorated.

13. The method of claim 12, wherein said corneoscleral disease, disorder or condition is selected from the group consisting of keratitis, episcleritis, scleritis, corneal ulceration, sequelae of corneal ulceration, corneal ectasia, acquired abnormalities of corneal shape, keratoconus, corneal astigmatism, keratoglobus, posterior corneal depressions, keratectasia, keratocele, descemetocele, pellucid marginal degeneration, Terrien's marginal dystrophy, Mooren's ulcers, central corneal ulcers, marginal corneal ulcers, staph marginal ulceration, Salzman's nodular dystrophy, age-related peripheral corneal atrophy, geographic ulceration, disciform stromal keratitis, metaherpetic ulceration, keratomalacia, post penetrating keratoplasty, incisional wounds, anterior membrane dystrophies, stromal dystrophies, ocular mucous membrane pemphigoid, necrotizing scleritis, scleromalacia, coloboma, myopia, presbyopia, hyperopia, scleral buckle induced scleromalacia, congenital hereditary stromal dystrophy, congenital anterior staphyloma, sclerocornea, traumatic breaks in Descemet's membrane, corneal keloids, scleral ectasia, scleral staphyloma, deep scleritis, necrotizing scleritis, scleromalacia perforans, hyaline degeneration of the sclera, paralimbal scleromalacia, ocular graft vs host disease, and choroideremia.

14. The method of claim 12, wherein said corneoscleral disease, disorder or condition is myopia, keratoconus or presbyopia.

15. The method of claim 12, wherein said method is used to treat or prevent myopia.

16. The method of claim 12, wherein said method is used to treat or prevent presbyopia.

17. The method of claim 12, wherein said composition is administered to the eye conjunctivally or subconjunctivally.

18. The method of claim 12, wherein said composition is administered to the eye in the form of one or more drops of solution or a suspension that contains the composition.

19. The method of claim 12, wherein said composition is administered to the eye via injection.

20. The method of claim 19, wherein said injection is intravitreal injection.

21. The method of claim 12, wherein said composition is administered to the eye in the form of a coating on a solid material that is implanted into an eye structure.

22. The method of claim 12, wherein said composition is administered to the eye in the form of a wafer, film, gel, mesh or patch.

23. The method of claim 12, wherein said composition is attached to one or more spheres or nanoparticles that are delivered to or into an eye structure.

24. A medical device suitable for treating or preventing a corneoscleral disease, disorder or condition in a human or veterinary animal in need of treatment or prevention thereof, wherein said device comprises the composition of claim 5.

25. The medical device of claim 24, wherein said device is selected from the group consisting of a stent, a shunt, a suture, an absorbable mesh, an absorbable patch, a drug-releasing wafer, a film, and an internal infusion pump.

26. A method of treating, ameliorating or preventing myopia or presbyopia in a human or veterinary animal in need of treatment or prevention thereof, said method comprising implanting the medical device of claim 24 into the eye of said human or veterinary animal, and monitoring the medical condition of said human or veterinary animal until said myopia, keratoconus or presbyopia is cured, ameliorated or prevented.


SEQUENCE LISTING (479 sequences)

SEQ ID NO: 1 - protein, 21 aa, synthetic construct
  REGION 1..21: note = Synthetic
PPGPPGPPGPPGPPGPPGPPG

SEQ ID NO: 2 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXPGXPG

SEQ ID NO: 3 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGPXG

SEQ ID NO: 4 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XPGXPGXPGXPGXPGXPGXPG

SEQ ID NO: 5 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGPXG

SEQ ID NO: 6 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGXXGXXG

SEQ ID NO: 7 - protein, 21 aa, synthetic construct
  REGION 1..21: note = Synthetic
  MOD_RES 1: note = Chloroproline
  MOD_RES 4: note = Chloroproline
  MOD_RES 7: note = Chloroproline
  MOD_RES 10: note = Chloroproline
  MOD_RES 13: note = Chloroproline
  MOD_RES 16: note = Chloroproline
  MOD_RES 19: note = Chloroproline
XPGXPGXPGXPGXPGXPGXPG

SEQ ID NO: 8 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGPXG

SEQ ID NO: 9 - protein, 22 aa, synthetic construct
  REGION 1..22: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
  MOD_RES 21: note = Chloroproline
XXGXXGXXGXXGXXGXXGXXXG

SEQ ID NO: 10 - protein, 21 aa, synthetic construct
  REGION 1..21: note = Synthetic
PPGPPGCPGPPGPPGPPGPPG

SEQ ID NO: 11 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGCPGXPGXPGXPGXPG

SEQ ID NO: 12 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPCGPXGPXGPXGPXG

SEQ ID NO: 13 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPCGPXGPXGPXGPXG

SEQ ID NO: 14 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPCGPXGPXGPXGPXG

SEQ ID NO: 15 - protein, 21 aa, synthetic construct
  REGION 1..21: note = Synthetic
CPGPPGPPGPPGPPGPPGPPG

SEQ ID NO: 16 - protein, 21 aa, synthetic construct
  REGION 1..21: note = Synthetic
PCGPPGPPGPPGPPGPPGPPG

SEQ ID NO: 17 - protein, 21 aa, synthetic construct
  REGION 1..21: note = Synthetic
PPGCPGPPGPPGPPGPPGPPG

SEQ ID NO: 18 - protein, 21 aa, synthetic construct
  REGION 1..21: note = Synthetic
PPGPCGPPGPPGPPGPPGPPG

SEQ ID NO: 19 - protein, 21 aa, synthetic construct
  REGION 1..21: note = Synthetic
PPGPPGPCGPPGPPGPPGPPG

SEQ ID NO: 20 - protein, 21 aa, synthetic construct
  REGION 1..21: note = Synthetic
PPGPPGPPGCPGPPGPPGPPG

SEQ ID NO: 21 - protein, 21 aa, synthetic construct
  REGION 1..21: note = Synthetic
PPGPPGPPGPCGPPGPPGPPG

SEQ ID NO: 22 - protein, 21 aa, synthetic construct
  REGION 1..21: note = Synthetic
PPGPPGPPGPPGCPGPPGPPG

SEQ ID NO: 23 - protein, 21 aa, synthetic construct
  REGION 1..21: note = Synthetic
PPGPPGPPGPPGPCGPPGPPG

SEQ ID NO: 24 - protein, 21 aa, synthetic construct
  REGION 1..21: note = Synthetic
PPGPPGPPGPPGPPGCPGPPG

SEQ ID NO: 25 - protein, 21 aa, synthetic construct
  REGION 1..21: note = Synthetic
PPGPPGPPGPPGPPGPCGCPG

SEQ ID NO: 26 - protein, 21 aa, synthetic construct
  REGION 1..21: note = Synthetic
PPGPPGPPGPPGPPGPPGCPG

SEQ ID NO: 27 - protein, 21 aa, synthetic construct
  REGION 1..21: note = Synthetic
PPGPPGPPGPPGPPGPPGPCG

SEQ ID NO: 28 - protein, 21 aa, synthetic construct
  REGION 1..21: note = Synthetic
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
CPGXPGXPGXPGXPGXPGXPG

SEQ ID NO: 29 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XCGXPGXPGXPGXPGXPGXPG

SEQ ID NO: 30 - protein, 21 aa, synthetic construct
  REGION 1..21: note = Synthetic
  MOD_RES 1: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGCPGXPGXPGXPGXPGXPG

SEQ ID NO: 31 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXCGXPGXPGXPGXPGXPG

SEQ ID NO: 32 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXCGXPGXPGXPGXPG

SEQ ID NO: 33 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGCPGXPGXPGXPG

SEQ ID NO: 34 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXCGXPGXPGXPG

SEQ ID NO: 35 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 16: note = 4(R)-hydroxyproline
  MOD_RES 19: note = 4(R)-hydroxyproline
XPGXPGXPGXPGCPGXPGXPG

SEQ ID NO: 36 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXCGXPGXPG

SEQ ID NO: 37 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 19: note = 4(R)-hydroxyproline
XPGXPGXPGXPGXPGCPGXPG

SEQ ID NO: 38 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXCGXPG

SEQ ID NO: 39 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
XPGXPGXPGXPGXPGXPGCPG

SEQ ID NO: 40 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXPGXCG

SEQ ID NO: 41 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
CXGPXGPXGPXGPXGPXGPXG

SEQ ID NO: 42 - protein, 21 aa, synthetic construct
  REGION 1..21: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PCGPXGPXGPXGPXGPXGPXG

SEQ ID NO: 43 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGCXGPXGPXGPXGPXGPXG

SEQ ID NO: 44 - protein, 21 aa, synthetic construct
  REGION 1..21: note = Synthetic
  MOD_RES 2: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPCGPXGPXGPXGPXGPXG

SEQ ID NO: 45 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGCXGPXGPXGPXGPXG

SEQ ID NO: 46 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGCXGPXGPXGPXG

SEQ ID NO: 47 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPCGPXGPXGPXG

SEQ ID NO: 48 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGCXGPXGPXG

SEQ ID NO: 49 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 17: note = 4(R)-hydroxyproline
  MOD_RES 20: note = 4(R)-hydroxyproline
PXGPXGPXGPXGPCGPXGPXG

SEQ ID NO: 50 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGCXGPXG

SEQ ID NO: 51 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 20: note = 4(R)-hydroxyproline
PXGPXGPXGPXGPXGPCGPXG

SEQ ID NO: 52 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGCXG

SEQ ID NO: 53 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
PXGPXGPXGPXGPXGPXGPCG

SEQ ID NO: 54 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
CXGPXGPXGPXGPXGPXGPXG

SEQ ID NO: 55 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PCGPXGPXGPXGPXGPXGPXG

SEQ ID NO: 56 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGCXGPXGPXGPXGPXGPXG

SEQ ID NO: 57 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPCGPXGPXGPXGPXGPXG

SEQ ID NO: 58 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGCXGPXGPXGPXGPXG

SEQ ID NO: 59 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGCXGPXGPXGPXG

SEQ ID NO: 60 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPCGPXGPXGPXG

SEQ ID NO: 61 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGCXGPXGPXG

SEQ ID NO: 62 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPCGPXGPXG

SEQ ID NO: 63 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGCXGPXG

SEQ ID NO: 64 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPCGPXG

SEQ ID NO: 65 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGCXG

SEQ ID NO: 66 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPCG

SEQ ID NO: 67 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
CXGPXGPXGPXGPXGPXGPXG

SEQ ID NO: 68 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PCGPXGPXGPXGPXGPXGPXG

SEQ ID NO: 69 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGCXGPXGPXGPXGPXGPXG

SEQ ID NO: 70 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPCGPXGPXGPXGPXGPXG

SEQ ID NO: 71 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGCXGPXGPXGPXGPXG

SEQ ID NO: 72 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPCGPXGPXGPXGPXG

SEQ ID NO: 73 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGCXGPXGPXGPXG

SEQ ID NO: 74 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPCGPXGPXGPXG

SEQ ID NO: 75 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGCXGPXGPXG

SEQ ID NO: 76 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPCGPXGPXG

SEQ ID NO: 77 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGCXGPXG

SEQ ID NO: 78 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPCGPXG

SEQ ID NO: 79 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGCXG

SEQ ID NO: 80 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPCG

SEQ ID NO: 81 - protein, 21 aa, synthetic construct
  REGION 1..21: note = Synthetic
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
CXGXXGXXGXXGXXGXXGXXG

SEQ ID NO: 82 - protein, 21 aa, synthetic construct
  REGION 1..21: note = Synthetic
  MOD_RES 1: note = Fluoroproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XCGXXGXXGXXGXXGXXGXXG

SEQ ID NO: 83 - protein, 21 aa, synthetic construct
  REGION 1..21: note = Synthetic
  MOD_RES 1: note = Fluoroproline
  MOD_RES 2: note = 4(R)-hydroxyproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGCXGXXGXXGXXGXXGXXG

SEQ ID NO: 84 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXCGXXGXXGXXGXXGXXG

SEQ ID NO: 85 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGCXGXXGXXGXXGXXG

SEQ ID NO: 86 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXCGXXGXXGXXGXXG

SEQ ID NO: 87 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGCXGXXGXXGXXG

SEQ ID NO: 88 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXCGXXGXXGXXG

SEQ ID NO: 89 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGCXGXXGXXG

SEQ ID NO: 90 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXCGXXGXXG

SEQ ID NO: 91 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGCXGXXG

SEQ ID NO: 92 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 19: note = Fluoroproline
  MOD_RES 20: note = 4(R)-hydroxyproline
XXGXXGXXGXXGXXGXCGXXG

SEQ ID NO: 93 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 20: note = 4(R)-hydroxyproline
XXGXXGXXGXXGXXGXXGCXG

SEQ ID NO: 94 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
XXGXXGXXGXXGXXGXXGXCG

SEQ ID NO: 95 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
CXGPXGPXGPXGPXGPXGPXG

SEQ ID NO: 96 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PCGPXGPXGPXGPXGPXGPXG

SEQ ID NO: 97 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGCXGPXGPXGPXGPXGPXG

SEQ ID NO: 98 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPCGPXGPXGPXGPXGPXG

SEQ ID NO: 99 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGCXGPXGPXGPXGPXG

SEQ ID NO: 100 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGCXGPXGPXGPXG

SEQ ID NO: 101 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPCGPXGPXGPXG

SEQ ID NO: 102 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGCXGPXGPXG

SEQ ID NO: 103 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPCGPXGPXG

SEQ ID NO: 104 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGCXGPXG

SEQ ID NO: 105 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPCGPXG

SEQ ID NO: 106 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGCXG

SEQ ID NO: 107 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPCG

SEQ ID NO: 108 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
CXGPXGPXGPXGPXGPXGPXG

SEQ ID NO: 109 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PCGPXGPXGPXGPXGPXGPXG

SEQ ID NO: 110 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGCXGPXGPXGPXGPXGPXG

SEQ ID NO: 111 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPCGPXGPXGPXGPXGPXG

SEQ ID NO: 112 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGCXGPXGPXGPXGPXG

SEQ ID NO: 113 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPCGPXGPXGPXGPXG

SEQ ID NO: 114 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGCXGPXGPXGPXG

SEQ ID NO: 115 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPCGPXGPXGPXG

SEQ ID NO: 116 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGCXGPXGPXG

SEQ ID NO: 117 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPCGPXGPXG

SEQ ID NO: 118 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGCXGPXG

SEQ ID NO: 119 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPCGPXG

SEQ ID NO: 120 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGCXG

SEQ ID NO: 121 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPCG

SEQ ID NO: 122 - protein, 21 aa, synthetic construct
  REGION 1..21: note = Synthetic
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
CXGXXGXXGXXGXXGXXGXXG

SEQ ID NO: 123 - protein, 21 aa, synthetic construct
  REGION 1..21: note = Synthetic
  MOD_RES 1: note = Chloroproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XCGXXGXXGXXGXXGXXGXXG

SEQ ID NO: 124 - protein, 21 aa, synthetic construct
  REGION 1..21: note = Synthetic
  MOD_RES 1: note = Chloroproline
  MOD_RES 2: note = 4(R)-hydroxyproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGCXGXXGXXGXXGXXGXXG

SEQ ID NO: 125 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXCGXXGXXGXXGXXGXXG

SEQ ID NO: 126 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGCXGXXGXXGXXGXXG

SEQ ID NO: 127 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXCGXXGXXGXXGXXG

SEQ ID NO: 128 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGCXGXXGXXGXXG

SEQ ID NO: 129 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXCGXXGXXGXXG

SEQ ID NO: 130 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGCXGXXGXXG

SEQ ID NO: 131 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGXCGXXGXXG

SEQ ID NO: 132 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 17: note = 4(R)-hydroxyproline
  MOD_RES 19: note = Chloroproline
  MOD_RES 20: note = 4(R)-hydroxyproline
XXGXXGXXGXXGXXGCXGXXG

SEQ ID NO: 133 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 19: note = Chloroproline
  MOD_RES 20: note = 4(R)-hydroxyproline
XXGXXGXXGXXGXXGXCGXXG

SEQ ID NO: 134 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 20: note = 4(R)-hydroxyproline
XXGXXGXXGXXGXXGXXGCXG

SEQ ID NO: 135 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
XXGXXGXXGXXGXXGXXGXCG

SEQ ID NO: 136 - protein, 21 aa, synthetic construct
  REGION 1..21: note = Synthetic
PPGPPGMPGPPGPPGPPGPPG

SEQ ID NO: 137 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGMPGXPGXPGXPGXPG

SEQ ID NO: 138 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPMGPXGPXGPXGPXG

SEQ ID NO: 139 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPMGPXGPXGPXGPXG

SEQ ID NO: 140 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPMGPXGPXGPXGPXG

SEQ ID NO: 141 - protein, 21 aa, synthetic construct
  REGION 1..21: note = Synthetic
MPGPPGPPGPPGPPGPPGPPG

SEQ ID NO: 142 - protein, 21 aa, synthetic construct
  REGION 1..21: note = Synthetic
PMGPPGPPGPPGPPGPPGPPG

SEQ ID NO: 143 - protein, 21 aa, synthetic construct
  REGION 1..21: note = Synthetic
PPGMPGPPGPPGPPGPPGPPG

SEQ ID NO: 144 - protein, 21 aa, synthetic construct
  REGION 1..21: note = Synthetic
PPGPMGPPGPPGPPGPPGPPG

SEQ ID NO: 145 - protein, 21 aa, synthetic construct
  REGION 1..21: note = Synthetic
PPGPPGPMGPPGPPGPPGPPG

SEQ ID NO: 146 - protein, 21 aa, synthetic construct
  REGION 1..21: note = Synthetic
PPGPPGPPGMPGPPGPPGPPG

SEQ ID NO: 147 - protein, 21 aa, synthetic construct
  REGION 1..21: note = Synthetic
PPGPPGPPGPMGPPGPPGPPG

SEQ ID NO: 148 - protein, 21 aa, synthetic construct
  REGION 1..21: note = Synthetic
PPGPPGPPGPPGMPGPPGPPG

SEQ ID NO: 149 - protein, 21 aa, synthetic construct
  REGION 1..21: note = Synthetic
PPGPPGPPGPPGPMGPPGPPG

SEQ ID NO: 150 - protein, 21 aa, synthetic construct
  REGION 1..21: note = Synthetic
PPGPPGPPGPPGPPGMPGPPG

SEQ ID NO: 151 - protein, 21 aa, synthetic construct
  REGION 1..21: note = Synthetic
PPGPPGPPGPPGPPGPMGPPG

SEQ ID NO: 152 - protein, 21 aa, synthetic construct
  REGION 1..21: note = Synthetic
PPGPPGPPGPPGPPGPPGMPG

SEQ ID NO: 153 - protein, 21 aa, synthetic construct
  REGION 1..21: note = Synthetic
PPGPPGPPGPPGPPGPPGPMG

SEQ ID NO: 154 - protein, 21 aa, synthetic construct
  REGION 1..21: note = Synthetic
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
MPGXPGXPGXPGXPGXPGXPG

SEQ ID NO: 155 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XMGXPGXPGXPGXPGXPGXPG

SEQ ID NO: 156 - protein, 21 aa, synthetic construct
  REGION 1..21: note = Synthetic
  MOD_RES 1: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGMPGXPGXPGXPGXPGXPG

SEQ ID NO: 157 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXMGXPGXPGXPGXPGXPG

SEQ ID NO: 158 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXMGXPGXPGXPGXPG

SEQ ID NO: 159 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGMPGXPGXPGXPG

SEQ ID NO: 160 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXMGXPGXPGXPG

SEQ ID NO: 161 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 16: note = 4(R)-hydroxyproline
  MOD_RES 19: note = 4(R)-hydroxyproline
XPGXPGXPGXPGMPGXPGXPG

SEQ ID NO: 162 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXMGXPGXPG

SEQ ID NO: 163 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 19: note = 4(R)-hydroxyproline
XPGXPGXPGXPGXPGMPGXPG

SEQ ID NO: 164 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXMGXPG

SEQ ID NO: 165 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
XPGXPGXPGXPGXPGXPGMPG

SEQ ID NO: 166 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXPGXMG

SEQ ID NO: 167 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
MXGPXGPXGPXGPXGPXGPXG

SEQ ID NO: 168 - protein, 21 aa, synthetic construct
  REGION 1..21: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PMGPXGPXGPXGPXGPXGPXG

SEQ ID NO: 169 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGMXGPXGPXGPXGPXGPXG

SEQ ID NO: 170 - protein, 21 aa, synthetic construct
  REGION 1..21: note = Synthetic
  MOD_RES 2: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPMGPXGPXGPXGPXGPXG

SEQ ID NO: 171 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGMXGPXGPXGPXGPXG

SEQ ID NO: 172 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGMXGPXGPXGPXG

SEQ ID NO: 173 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPMGPXGPXGPXG

SEQ ID NO: 174 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGMXGPXGPXG

SEQ ID NO: 175 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 17: note = 4(R)-hydroxyproline
  MOD_RES 20: note = 4(R)-hydroxyproline
PXGPXGPXGPXGPMGPXGPXG

SEQ ID NO: 176 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGMXGPXG

SEQ ID NO: 177 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 20: note = 4(R)-hydroxyproline
PXGPXGPXGPXGPXGPMGPXG

SEQ ID NO: 178 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGMXG

SEQ ID NO: 179 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
PXGPXGPXGPXGPXGPXGPMG

SEQ ID NO: 180 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
MXGPXGPXGPXGPXGPXGPXG

SEQ ID NO: 181 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PMGPXGPXGPXGPXGPXGPXG

SEQ ID NO: 182 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGMXGPXGPXGPXGPXGPXG

SEQ ID NO: 183 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPMGPXGPXGPXGPXGPXG

SEQ ID NO: 184 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGMXGPXGPXGPXGPXG

SEQ ID NO: 185 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGMXGPXGPXGPXG

SEQ ID NO: 186 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPMGPXGPXGPXG

SEQ ID NO: 187 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGMXGPXGPXG

SEQ ID NO: 188 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPMGPXGPXG

SEQ ID NO: 189 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGMXGPXG

SEQ ID NO: 190 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPMGPXG

SEQ ID NO: 191 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGMXG

SEQ ID NO: 192 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPMG

SEQ ID NO: 193 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
MXGPXGPXGPXGPXGPXGPXG

SEQ ID NO: 194 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PMGPXGPXGPXGPXGPXGPXG

SEQ ID NO: 195 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGMXGPXGPXGPXGPXGPXG

SEQ ID NO: 196 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPMGPXGPXGPXGPXGPXG

SEQ ID NO: 197 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGMXGPXGPXGPXGPXG

SEQ ID NO: 198 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPMGPXGPXGPXGPXG

SEQ ID NO: 199 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGMXGPXGPXGPXG

SEQ ID NO: 200 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPMGPXGPXGPXG

SEQ ID NO: 201 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGMXGPXGPXG

SEQ ID NO: 202 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPMGPXGPXG

SEQ ID NO: 203 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGMXGPXG

SEQ ID NO: 204 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPMGPXG

SEQ ID NO: 205 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGMXG

SEQ ID NO: 206 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPMG

SEQ ID NO: 207 - protein, 21 aa, synthetic construct
  REGION 1..21: note = Synthetic
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
MXGXXGXXGXXGXXGXXGXXG

SEQ ID NO: 208 - protein, 21 aa, synthetic construct
  REGION 1..21: note = Synthetic
  MOD_RES 1: note = fluoroproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XMGXXGXXGXXGXXGXXGXXG

SEQ ID NO: 209 - protein, 21 aa, synthetic construct
  REGION 1..21: note = Synthetic
  MOD_RES 1: note = fluoroproline
  MOD_RES 2: note = 4(R)-hydroxyproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGMXGXXGXXGXXGXXGXXG

SEQ ID NO: 210 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXMGXXGXXGXXGXXGXXG

SEQ ID NO: 211 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGMXGXXGXXGXXGXXG

SEQ ID NO: 212 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGXMGXXGXXGXXGXXG

SEQ ID NO: 213 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGXXGMXGXXGXXGXXG

SEQ ID NO: 214 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGXXGXMGXXGXXGXXG

SEQ ID NO: 215 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGXXGXXGMXGXXGXXG

SEQ ID NO: 216 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 16: note = fluoroproline
  MOD_RES 17: note = 4(R)-hydroxyproline
  MOD_RES 19: note = fluoroproline
  MOD_RES 20: note = 4(R)-hydroxyproline
XXGXXGXXGXXGXMGXXGXXG

SEQ ID NO: 217 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 17: note = 4(R)-hydroxyproline
  MOD_RES 19: note = fluoroproline
  MOD_RES 20: note = 4(R)-hydroxyproline
XXGXXGXXGXXGXXGMXGXXG

SEQ ID NO: 218 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 19: note = fluoroproline
  MOD_RES 20: note = 4(R)-hydroxyproline
XXGXXGXXGXXGXXGXMGXXG

SEQ ID NO: 219 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 20: note = 4(R)-hydroxyproline
XXGXXGXXGXXGXXGXXGMXG

SEQ ID NO: 220 - protein, 21 aa, synthetic construct
  REGION 1..21: note = Synthetic
  MOD_RES 1: note = fluoroproline
  MOD_RES 2: note = 4(R)-hydroxyproline
  MOD_RES 4: note = fluoroproline
  MOD_RES 5: note = 4(R)-hydroxyproline
  MOD_RES 7: note = fluoroproline
  MOD_RES 8: note = 4(R)-hydroxyproline
  MOD_RES 10: note = fluoroproline
  MOD_RES 11: note = 4(R)-hydroxyproline
  MOD_RES 13: note = fluoroproline
  MOD_RES 14: note = 4(R)-hydroxyproline
  MOD_RES 16: note = fluoroproline
  MOD_RES 17: note = 4(R)-hydroxyproline
  MOD_RES 19: note = fluoroproline
XXGXXGXXGXXGXXGXXGXMG

SEQ ID NO: 221 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
MXGPXGPXGPXGPXGPXGPXG

SEQ ID NO: 222 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PMGPXGPXGPXGPXGPXGPXG

SEQ ID NO: 223 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGMXGPXGPXGPXGPXGPXG

SEQ ID NO: 224 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPMGPXGPXGPXGPXGPXG

SEQ ID NO: 225 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGMXGPXGPXGPXGPXG

SEQ ID NO: 226 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGMXGPXGPXGPXG

SEQ ID NO: 227 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPMGPXGPXGPXG

SEQ ID NO: 228 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGMXGPXGPXG

SEQ ID NO: 229 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPMGPXGPXG

SEQ ID NO: 230 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGMXGPXG

SEQ ID NO: 231 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPMGPXG

SEQ ID NO: 232 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGMXG

SEQ ID NO: 233 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPMG

SEQ ID NO: 234 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
MXGPXGPXGPXGPXGPXGPXG

SEQ ID NO: 235 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PMGPXGPXGPXGPXGPXGPXG

SEQ ID NO: 236 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGMXGPXGPXGPXGPXGPXG

SEQ ID NO: 237 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPMGPXGPXGPXGPXGPXG

SEQ ID NO: 238 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGMXGPXGPXGPXGPXG

SEQ ID NO: 239 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPMGPXGPXGPXGPXG

SEQ ID NO: 240 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGMXGPXGPXGPXG

SEQ ID NO: 241 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPMGPXGPXGPXG

SEQ ID NO: 242 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGMXGPXGPXG

SEQ ID NO: 243 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPMGPXGPXG

SEQ ID NO: 244 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGMXGPXG

SEQ ID NO: 245 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPMGPXG

SEQ ID NO: 246 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGMXG

SEQ ID NO: 247 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPMG

SEQ ID NO: 248 - protein, 21 aa, synthetic construct
  REGION 1..21: note = Synthetic
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
MXGXXGXXGXXGXXGXXGXXG

SEQ ID NO: 249 - protein, 21 aa, synthetic construct
  REGION 1..21: note = Synthetic
  MOD_RES 1: note = Chloroproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XMGXXGXXGXXGXXGXXGXXG

SEQ ID NO: 250 - protein, 21 aa, synthetic construct
  REGION 1..21: note = Synthetic
  MOD_RES 1: note = Chloroproline
  MOD_RES 2: note = 4(R)-hydroxyproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGMXGXXGXXGXXGXXGXXG

SEQ ID NO: 251 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXMGXXGXXGXXGXXGXXG

SEQ ID NO: 252 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGMXGXXGXXGXXGXXG

SEQ ID NO: 253 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXMGXXGXXGXXGXXG

SEQ ID NO: 254 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGMXGXXGXXGXXG

SEQ ID NO: 255 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXMGXXGXXGXXG

SEQ ID NO: 256 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGMXGXXGXXG

SEQ ID NO: 257 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGXMGXXGXXG

SEQ ID NO: 258 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 17: note = 4(R)-hydroxyproline
  MOD_RES 19: note = Chloroproline
  MOD_RES 20: note = 4(R)-hydroxyproline
XXGXXGXXGXXGXXGMXGXXG

SEQ ID NO: 259 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 19: note = Chloroproline
  MOD_RES 20: note = 4(R)-hydroxyproline
XXGXXGXXGXXGXXGXMGXXG

SEQ ID NO: 260 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 20: note = 4(R)-hydroxyproline
XXGXXGXXGXXGXXGXXGMXG

SEQ ID NO: 261 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
XXGXXGXXGXXGXXGXXGXMG

SEQ ID NO: 262 - protein, 21 aa, synthetic construct
  REGION 1..21: note = Synthetic
PPGPPGKPGPPGPPGPPGPPG

SEQ ID NO: 263 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGKPGXPGXPGXPGXPG

SEQ ID NO: 264 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPKGPXGPXGPXGPXG

SEQ ID NO: 265 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPKGPXGPXGPXGPXG

SEQ ID NO: 266 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPKGPXGPXGPXGPXG

SEQ ID NO: 267 - protein, 21 aa, synthetic construct
  REGION 1..21: note = Synthetic
KPGPPGPPGPPGPPGPPGPPG

SEQ ID NO: 268 - protein, 21 aa, synthetic construct
  REGION 1..21: note = Synthetic
PKGPPGPPGPPGPPGPPGPPG

SEQ ID NO: 269 - protein, 21 aa, synthetic construct
  REGION 1..21: note = Synthetic
PPGKPGPPGPPGPPGPPGPPG

SEQ ID NO: 270 - protein, 21 aa, synthetic construct
  REGION 1..21: note = Synthetic
PPGPKGPPGPPGPPGPPGPPG

SEQ ID NO: 271 - protein, 21 aa, synthetic construct
  REGION 1..21: note = Synthetic
PPGPPGPKGPPGPPGPPGPPG

SEQ ID NO: 272 - protein, 21 aa, synthetic construct
  REGION 1..21: note = Synthetic
PPGPPGPPGKPGPPGPPGPPG

SEQ ID NO: 273 - protein, 21 aa, synthetic construct
  REGION 1..21: note = Synthetic
PPGPPGPPGPKGPPGPPGPPG

SEQ ID NO: 274 - protein, 21 aa, synthetic construct
  REGION 1..21: note = Synthetic
PPGPPGPPGPPGKPGPPGPPG

SEQ ID NO: 275 - protein, 21 aa, synthetic construct
  REGION 1..21: note = Synthetic
PPGPPGPPGPPGPKGPPGPPG

SEQ ID NO: 276 - protein, 21 aa, synthetic construct
  REGION 1..21: note = Synthetic
PPGPPGPPGPPGPPGKPGPPG

SEQ ID NO: 277 - protein, 21 aa, synthetic construct
  REGION 1..21: note = Synthetic
PPGPPGPPGPPGPPGPKGPPG

SEQ ID NO: 278 - protein, 21 aa, synthetic construct
  REGION 1..21: note = Synthetic
PPGPPGPPGPPGPPGPPGKPG

SEQ ID NO: 279 - protein, 21 aa, synthetic construct
  REGION 1..21: note = Synthetic
PPGPPGPPGPPGPPGPPGPKG

SEQ ID NO: 280 - protein, 21 aa, synthetic construct
  REGION 1..21: note = Synthetic
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
KPGXPGXPGXPGXPGXPGXPG

SEQ ID NO: 281 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XKGXPGXPGXPGXPGXPGXPG

SEQ ID NO: 282 - protein, 21 aa, synthetic construct
  REGION 1..21: note = Synthetic
  MOD_RES 1: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGKPGXPGXPGXPGXPGXPG

SEQ ID NO: 283 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXKGXPGXPGXPGXPGXPG

SEQ ID NO: 284 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXKGXPGXPGXPGXPG

SEQ ID NO: 285 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGKPGXPGXPGXPG

SEQ ID NO: 286 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXKGXPGXPGXPG

SEQ ID NO: 287 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 16: note = 4(R)-hydroxyproline
  MOD_RES 19: note = 4(R)-hydroxyproline
XPGXPGXPGXPGKPGXPGXPG

SEQ ID NO: 288 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXKGXPGXPG

SEQ ID NO: 289 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 19: note = 4(R)-hydroxyproline
XPGXPGXPGXPGXPGKPGXPG

SEQ ID NO: 290 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXKGXPG

SEQ ID NO: 291 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
XPGXPGXPGXPGXPGXPGKPG

SEQ ID NO: 292 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXPGXKG

SEQ ID NO: 293 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
KXGPXGPXGPXGPXGPXGPXG

SEQ ID NO: 294 - protein, 21 aa, synthetic construct
  REGION 1..21: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PKGPXGPXGPXGPXGPXGPXG

SEQ ID NO: 295 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGKXGPXGPXGPXGPXGPXG

SEQ ID NO: 296 - protein, 21 aa, synthetic construct
  REGION 1..21: note = Synthetic
  MOD_RES 2: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPKGPXGPXGPXGPXGPXG

SEQ ID NO: 297 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGKXGPXGPXGPXGPXG

SEQ ID NO: 298 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGKXGPXGPXGPXG

SEQ ID NO: 299 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPKGPXGPXGPXG

SEQ ID NO: 300 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGKXGPXGPXG

SEQ ID NO: 301 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 17: note = 4(R)-hydroxyproline
  MOD_RES 20: note = 4(R)-hydroxyproline
PXGPXGPXGPXGPKGPXGPXG

SEQ ID NO: 302 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGKXGPXG

SEQ ID NO: 303 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 20: note = 4(R)-hydroxyproline
PXGPXGPXGPXGPXGPKGPXG

SEQ ID NO: 304 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGKXG

SEQ ID NO: 305 - protein, 21 aa, synthetic construct
  REGION 1..21: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
PXGPXGPXGPXGPXGPXGPKG

SEQ ID NO: 306 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
KXGPXGPXGPXGPXGPXGPXG

SEQ ID NO: 307 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PKGPXGPXGPXGPXGPXGPXG

SEQ ID NO: 308 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGKXGPXGPXGPXGPXGPXG

SEQ ID NO: 309 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPKGPXGPXGPXGPXGPXG

SEQ ID NO: 310 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGKXGPXGPXGPXGPXG

SEQ ID NO: 311 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGKXGPXGPXGPXG

SEQ ID NO: 312 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPKGPXGPXGPXG

SEQ ID NO: 313 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGKXGPXGPXG

SEQ ID NO: 314 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPKGPXGPXG

SEQ ID NO: 315 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGKXGPXG

SEQ ID NO: 316 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPKGPXG

SEQ ID NO: 317 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGKXG

SEQ ID NO: 318 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPKG

SEQ ID NO: 319 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
KXGPXGPXGPXGPXGPXGPXG

SEQ ID NO: 320 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PKGPXGPXGPXGPXGPXGPXG

SEQ ID NO: 321 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGKXGPXGPXGPXGPXGPXG

SEQ ID NO: 322 - protein, 21 aa, synthetic construct
  REGION 1..21: note = Synthetic
  MOD_RES 2: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPKGPXGPXGPXGPXGPXG

SEQ ID NO: 323 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGKXGPXGPXGPXGPXG

SEQ ID NO: 324 - protein, 21 aa, synthetic construct
  REGION 1..21: note = Synthetic
  MOD_RES 2: note = Fluoroproline
  MOD_RES 5: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPKGPXGPXGPXGPXG

SEQ ID NO: 325 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGKXGPXGPXGPXG

SEQ ID NO: 326 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 14: note = Fluoroproline
  MOD_RES 17: note = Fluoroproline
  MOD_RES 20: note = Fluoroproline
PXGPXGPXGPKGPXGPXGPXG

SEQ ID NO: 327 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGKXGPXGPXG

SEQ ID NO: 328 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 17: note = Fluoroproline
  MOD_RES 20: note = Fluoroproline
PXGPXGPXGPXGPKGPXGPXG

SEQ ID NO: 329 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGKXGPXG

SEQ ID NO: 330 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 20: note = Fluoroproline
PXGPXGPXGPXGPXGPKGPXG

SEQ ID NO: 331 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGKXG

SEQ ID NO: 332 - protein, 21 aa, synthetic construct
  REGION 1..21: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGPKG

SEQ ID NO: 333 - protein, 21 aa, synthetic construct
  REGION 1..21: note = Synthetic
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
KXGXXGXXGXXGXXGXXGXXG

SEQ ID NO: 334 - protein, 21 aa, synthetic construct
  REGION 1..21: note = Synthetic
  MOD_RES 1: note = Fluoroproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XKGXXGXXGXXGXXGXXGXXG

SEQ ID NO: 335 - protein, 21 aa, synthetic construct
  REGION 1..21: note = Synthetic
  MOD_RES 1: note = Fluoroproline
  MOD_RES 2: note = 4(R)-hydroxyproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGKXGXXGXXGXXGXXGXXG

SEQ ID NO: 336 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXKGXXGXXGXXGXXGXXG

SEQ ID NO: 337 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGKXGXXGXXGXXGXXG

SEQ ID NO: 338 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXKGXXGXXGXXGXXG

SEQ ID NO: 339 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGKXGXXGXXGXXG

SEQ ID NO: 340 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXKGXXGXXGXXG

SEQ ID NO: 341 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGKXGXXGXXG

SEQ ID NO: 342 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXKGXXGXXG

SEQ ID NO: 343 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGKXGXXG

SEQ ID NO: 344 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 19: note = Fluoroproline
  MOD_RES 20: note = 4(R)-hydroxyproline
XXGXXGXXGXXGXXGXKGXXG

SEQ ID NO: 345 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 20: note = 4(R)-hydroxyproline
XXGXXGXXGXXGXXGXXGKXG

SEQ ID NO: 346 - protein, 21 aa, synthetic construct
  REGION 1..21: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
XXGXXGXXGXXGXXGXXGXKG

SEQ ID NO: 347 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
KXGPXGPXGPXGPXGPXGPXG

SEQ ID NO: 348 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PKGPXGPXGPXGPXGPXGPXG

SEQ ID NO: 349 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGKXGPXGPXGPXGPXGPXG

SEQ ID NO: 350 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPKGPXGPXGPXGPXGPXG

SEQ ID NO: 351 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGKXGPXGPXGPXGPXG

SEQ ID NO: 352 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGKXGPXGPXGPXG

SEQ ID NO: 353 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPKGPXGPXGPXG

SEQ ID NO: 354 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGKXGPXGPXG

SEQ ID NO: 355 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPKGPXGPXG

SEQ ID NO: 356 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGKXGPXG

SEQ ID NO: 357 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPKGPXG

SEQ ID NO: 358 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGKXG

SEQ ID NO: 359 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPKG

SEQ ID NO: 360 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
KXGPXGPXGPXGPXGPXGPXG

SEQ ID NO: 361 - protein, 21 aa, synthetic construct
  REGION 1..21: note = Synthetic
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PKGPXGPXGPXGPXGPXGPXG

SEQ ID NO: 362 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGKXGPXGPXGPXGPXGPXG

SEQ ID NO: 363 - protein, 21 aa, synthetic construct
  REGION 1..21: note = Synthetic
  MOD_RES 2: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPKGPXGPXGPXGPXGPXG

SEQ ID NO: 364 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGKXGPXGPXGPXGPXG

SEQ ID NO: 365 - protein, 21 aa, synthetic construct
  REGION 1..21: note = Synthetic
  MOD_RES 2: note = Chloroproline
  MOD_RES 5: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPKGPXGPXGPXGPXG

SEQ ID NO: 366 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGKXGPXGPXGPXG

SEQ ID NO: 367 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 14: note = Chloroproline
  MOD_RES 17: note = Chloroproline
  MOD_RES 20: note = Chloroproline
PXGPXGPXGPKGPXGPXGPXG

SEQ ID NO: 368 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGKXGPXGPXG

SEQ ID NO: 369 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 17: note = Chloroproline
  MOD_RES 20: note = Chloroproline
PXGPXGPXGPXGPKGPXGPXG

SEQ ID NO: 370 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGKXGPXG

SEQ ID NO: 371 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 20: note = Chloroproline
PXGPXGPXGPXGPXGPKGPXG

SEQ ID NO: 372 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline
PXGPXGPXGPXGPXGPXGKXG

SEQ ID NO: 373 - protein, 21 aa, synthetic construct
  REGION 1..21: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
PXGPXGPXGPXGPXGPXGPKG

SEQ ID NO: 374 - protein, 21 aa, synthetic construct
  REGION 1..21: note = Synthetic
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
KXGXXGXXGXXGXXGXXGXXG

SEQ ID NO: 375 - protein, 21 aa, synthetic construct
  REGION 1..21: note = Synthetic
  MOD_RES 1: note = Chloroproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XKGXXGXXGXXGXXGXXGXXG

SEQ ID NO: 376 - protein, 21 aa, synthetic construct
  REGION 1..21: note = Synthetic
  MOD_RES 1: note = Chloroproline
  MOD_RES 2: note = 4(R)-hydroxyproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGKXGXXGXXGXXGXXGXXG

SEQ ID NO: 377 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXKGXXGXXGXXGXXGXXG

SEQ ID NO: 378 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGKXGXXGXXGXXGXXG

SEQ ID NO: 379 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXKGXXGXXGXXGXXG

SEQ ID NO: 380 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGKXGXXGXXGXXG

SEQ ID NO: 381 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXKGXXGXXGXXG

SEQ ID NO: 382 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGKXGXXGXXG

SEQ ID NO: 383 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline
XXGXXGXXGXXGXKGXXGXXG

SEQ ID NO: 384 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 17: note = 4(R)-hydroxyproline
  MOD_RES 19: note = Chloroproline
  MOD_RES 20: note = 4(R)-hydroxyproline
XXGXXGXXGXXGXXGKXGXXG

SEQ ID NO: 385 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 19: note = Chloroproline
  MOD_RES 20: note = 4(R)-hydroxyproline
XXGXXGXXGXXGXXGXKGXXG

SEQ ID NO: 386 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 20: note = 4(R)-hydroxyproline
XXGXXGXXGXXGXXGXXGKXG

SEQ ID NO: 387 - protein, 21 aa, synthetic construct
  REGION 1..21: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
XXGXXGXXGXXGXXGXXGXKG

SEQ ID NO: 388 - protein, 20 aa, synthetic construct
  REGION 1..20: note = Synthetic
  MOD_RES 1: note = 4(R)-hydroxyproline
  MOD_RES 2: note = Fluoroproline
  MOD_RES 4: note = 4(R)-hydroxyproline
  MOD_RES 5: note = Fluoroproline
  MOD_RES 7: note = 4(R)-hydroxyproline
  MOD_RES 8: note = Fluoroproline
  MOD_RES 10: note = 4(R)-hydroxyproline
  MOD_RES 11: note = Fluoroproline
  MOD_RES 13: note = 4(R)-hydroxyproline
  MOD_RES 14: note = Fluoroproline
  MOD_RES 16: note = Fluoroproline
  MOD_RES 18: note = 4(R)-hydroxyproline
  MOD_RES 19: note = Fluoroproline
XXGXXGXXGXXGXXGXGXXG

SEQ ID NO: 389-395 - skipped (7 entries)

SEQ ID NO: 396 - protein, 30 aa, synthetic construct
  REGION 1..30: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
  MOD_RES 23: note = 4(R)-hydroxyproline
  MOD_RES 26: note = 4(R)-hydroxyproline
  MOD_RES 29: note = 4(R)-hydroxyproline
PXGPXGPXGPXGPXGPXGPXGPXGPXGPXG

SEQ ID NO: 397 - protein, 21 aa, synthetic construct
  REGION 1..21: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
GGGPXGPXGPXGPXGPXGPXG

SEQ ID NO: 398 - protein, 21 aa, synthetic construct
  REGION 1..21: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
GGGPXGPXGPXGPXGPXGPXG

SEQ ID NO: 399 - protein, 24 aa, synthetic construct
  REGION 1..24: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
  MOD_RES 23: note = 4(R)-hydroxyproline
GGGPXGPXGPXGPXGPXGPXGPXG

SEQ ID NO: 400 - protein, 24 aa, synthetic construct
  REGION 1..24: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
  MOD_RES 23: note = Fluoroproline
GGGPXGPXGPXGPXGPXGPXGPXG

SEQ ID NO: 401 - protein, 27 aa, synthetic construct
  REGION 1..27: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
  MOD_RES 23: note = 4(R)-hydroxyproline
  MOD_RES 26: note = 4(R)-hydroxyproline
GGGPXGPXGPXGPXGPXGPXGPXGPXG

SEQ ID NO: 402 - protein, 27 aa, synthetic construct
  REGION 1..27: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
  MOD_RES 23: note = Fluoroproline
  MOD_RES 26: note = Fluoroproline
GGGPXGPXGPXGPXGPXGPXGPXGPXG

SEQ ID NO: 403 - protein, 30 aa, synthetic construct
  REGION 1..30: note = Synthetic
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
  MOD_RES 23: note = 4(R)-hydroxyproline
  MOD_RES 26: note = 4(R)-hydroxyproline
  MOD_RES 29: note = 4(R)-hydroxyproline
GGGPXGPXGPXGPXGPXGPXGPXGPXGPXG

SEQ ID NO: 404 - protein, 30 aa, synthetic construct; identical to SEQ ID NO: 403
  REGION 1..30: note = Synthetic
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
  MOD_RES 23: note = Fluoroproline
  MOD_RES 26: note = Fluoroproline
  MOD_RES 29: note = Fluoroproline

SEQ ID NO: 405 - protein, 19 aa, synthetic construct
  REGION 1..19: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
PXGPXGPXGPXGPXGPXGY

SEQ ID NO: 406 - protein, 19 aa, synthetic construct
  REGION 1..19: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
PXGPXGPXGPXGPXGPXGY

SEQ ID NO: 407 - protein, 22 aa, synthetic construct
  REGION 1..22: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGPXGY

SEQ ID NO: 408 - protein, 22 aa, synthetic construct
  REGION 1..22: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
PXGPXGPXGPXGPXGPXGPXGY

SEQ ID NO: 409 - protein, 25 aa, synthetic construct
  REGION 1..25: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
  MOD_RES 23: note = 4(R)-hydroxyproline
PXGPXGPXGPXGPXGPXGPXGPXGY

SEQ ID NO: 410 - protein, 25 aa, synthetic construct
  REGION 1..25: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline
  MOD_RES 23: note = Fluoroproline
PXGPXGPXGPXGPXGPXGPXGPXGY

SEQ ID NO: 411 - protein, 10 aa, synthetic construct
  REGION 1..10: note = Synthetic
  MOD_RES 3: note = 4(R)-hydroxyproline
  MOD_RES 6: note = 4(R)-hydroxyproline
  MOD_RES 9: note = 4(R)-hydroxyproline
CPXGPXGPXG

SEQ ID NO: 412 - protein, 10 aa, synthetic construct
  REGION 1..10: note = Synthetic
  MOD_RES 3: note = Fluoroproline
  MOD_RES 6: note = Fluoroproline
  MOD_RES 9: note = Fluoroproline
CPXGPXGPXG

SEQ ID NO: 413 - protein, 16 aa, synthetic construct
  REGION 1..16: note = Synthetic
  MOD_RES 3: note = 4(R)-hydroxyproline
  MOD_RES 6: note = 4(R)-hydroxyproline
  MOD_RES 9: note = 4(R)-hydroxyproline
  MOD_RES 12: note = 4(R)-hydroxyproline
  MOD_RES 15: note = 4(R)-hydroxyproline
CPXGPXGPXGPXGPXG

SEQ ID NO: 414 - protein, 16 aa, synthetic construct
  REGION 1..16: note = Synthetic
  MOD_RES 3: note = Fluoroproline
  MOD_RES 6: note = Fluoroproline
  MOD_RES 9: note = Fluoroproline
  MOD_RES 12: note = Fluoroproline
  MOD_RES 15: note = Fluoroproline
CPXGPXGPXGPXGPXG

SEQ ID NO: 415 - protein, 22 aa, synthetic construct
  REGION 1..22: note = Synthetic
  MOD_RES 3: note = 4(R)-hydroxyproline
  MOD_RES 6: note = 4(R)-hydroxyproline
  MOD_RES 9: note = 4(R)-hydroxyproline
  MOD_RES 12: note = 4(R)-hydroxyproline
  MOD_RES 15: note = 4(R)-hydroxyproline
  MOD_RES 18: note = 4(R)-hydroxyproline
  MOD_RES 21: note = 4(R)-hydroxyproline
CPXGPXGPXGPXGPXGPXGPXG

SEQ ID NO: 416 - protein, 22 aa, synthetic construct
  REGION 1..22: note = Synthetic
  MOD_RES 3: note = Fluoroproline
  MOD_RES 6: note = Fluoroproline
  MOD_RES 9: note = Fluoroproline
  MOD_RES 12: note = Fluoroproline
  MOD_RES 15: note = Fluoroproline
  MOD_RES 18: note = Fluoroproline
  MOD_RES 21: note = Fluoroproline
CPXGPXGPXGPXGPXGPXGPXG

SEQ ID NO: 417 - protein, 60 aa, synthetic construct
  REGION 1..60: note = Synthetic
  VARIANT 1..2: note = X is proline, 4S-hydroxyproline, fluoroproline, chloroproline, lysine, cysteine or methionine
  VARIANT 4..5: note = X, if present, is proline, 4S-hydroxyproline, fluoroproline, chloroproline, lysine, cysteine or methionine
  VARIANT 7..8: note = X, if present, is proline, 4S-hydroxyproline, fluoroproline, chloroproline, lysine, cysteine or methionine
  VARIANT 10..11: note = X, if present, is proline, 4S-hydroxyproline, fluoroproline, chloroproline, lysine, cysteine or methionine
  VARIANT 13..14: note = X, if present, is proline, 4S-hydroxyproline, fluoroproline, chloroproline, lysine, cysteine or methionine
  VARIANT 16..17: note = X, if present, is proline, 4S-hydroxyproline, fluoroproline, chloroproline, lysine, cysteine or methionine
  VARIANT 19..20: note = X, if present, is proline, 4S-hydroxyproline, fluoroproline, chloroproline, lysine, cysteine or methionine
  VARIANT 22..23: note = X, if present, is proline, 4S-hydroxyproline, fluoroproline, chloroproline, lysine, cysteine or methionine
  VARIANT 25..26: note = X, if present, is proline, 4S-hydroxyproline, fluoroproline, chloroproline, lysine, cysteine or methionine
  VARIANT 28..29: note = X, if present, is proline, 4S-hydroxyproline, fluoroproline, chloroproline, lysine, cysteine or methionine
  VARIANT 31..32: note = X, if present, is proline, 4S-hydroxyproline, fluoroproline, chloroproline, lysine, cysteine or methionine
  VARIANT 34..35: note = X, if present, is proline, 4S-hydroxyproline, fluoroproline, chloroproline, lysine, cysteine or methionine
  VARIANT 37..38: note = X, if present, is proline, 4S-hydroxyproline, fluoroproline, chloroproline, lysine, cysteine or methionine
  VARIANT 40..41: note = X, if present, is proline, 4S-hydroxyproline, fluoroproline, chloroproline, lysine, cysteine or methionine
  VARIANT 43..44: note = X, if present, is proline, 4S-hydroxyproline, fluoroproline, chloroproline, lysine, cysteine or methionine
  VARIANT 46..47: note = X, if present, is proline, 4S-hydroxyproline, fluoroproline, chloroproline, lysine, cysteine or methionine
  VARIANT 49..50: note = X, if present, is proline, 4S-hydroxyproline, fluoroproline, chloroproline, lysine, cysteine or methionine
  VARIANT 52..53: note = X, if present, is proline, 4S-hydroxyproline, fluoroproline, chloroproline, lysine, cysteine or methionine
  VARIANT 55..56: note = X, if present, is proline, 4S-hydroxyproline, fluoroproline, chloroproline, lysine, cysteine or methionine
  VARIANT 58..59: note = X, if present, is proline, 4S-hydroxyproline, fluoroproline, chloroproline, lysine, cysteine or methionine
XXGXXGXXGXXGXXGXXGXXGXXGXXGXXGXXGXXGXXGXXGXXGXXGXXGXXGXXGXXG

SEQ ID NO: 418 - protein, 21 aa, synthetic construct
  REGION 1..21: note = Synthetic
PAGPAGPAGPAGPAGPAGPAG

SEQ ID NO: 419 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XAGXAGXAGXAGXAGXAGXAG

SEQ ID NO: 420 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XAGXAGXAGXAGXAGXAGXAG

SEQ ID NO: 421 - protein, 21 aa, synthetic construct
  REGION 1..21: note = Synthetic
PGEPGEPGEPGEPGEPGEPGE

SEQ ID NO: 422 - protein, 21 aa, synthetic construct
  REGION 1..21: note = Synthetic
PGQPGQPGQPGQPGQPGQPGQ

SEQ ID NO: 423 - protein, 21 aa, synthetic construct
  REGION 1..21: note = Synthetic
PGPPGPPGPPGPPGPPGPPGP

SEQ ID NO: 424 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGEXGEXGEXGEXGEXGEXGE

SEQ ID NO: 425 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGQXGQXGQXGQXGQXGQXGQ

SEQ ID NO: 426 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGEXGEXGEXGEXGEXGEXGE

SEQ ID NO: 427 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGQXGQXGQXGQXGQXGQXGQ

SEQ ID NO: 428 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGPXGPXGPXGPXGPXGPXGP

SEQ ID NO: 429 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGQXGQXGQXGQXGQXGQXGQ

SEQ ID NO: 430 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGEXGEXGEXGEXGEXGEXGE

SEQ ID NO: 431 - protein, 21 aa, synthetic construct
  REGION 1..21: note = Synthetic
PGDPGDPGDPGDPGDPGDPGD

SEQ ID NO: 432 - protein, 21 aa, synthetic construct
  REGION 1..21: note = Synthetic
PGNPGNPGNPGNPGNPGNPGN

SEQ ID NO: 433 - protein, 21 aa, synthetic construct
  REGION 1..21: note = Synthetic
KGQKGQKGQKGQKGQKGQKGQ

SEQ ID NO: 434 - protein, 21 aa, synthetic construct
  REGION 1..21: note = Synthetic
KGEKGEKGEKGEKGEKGEKGE

SEQ ID NO: 435 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XAGXAGXAGXAGXAGXAGXAG

SEQ ID NO: 436 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XAGXAGXAGXAGXAGXAGXAG

SEQ ID NO: 437 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGEXGEXGEXGEXGEXGEXGE

SEQ ID NO: 438 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGQXGQXGQXGQXGQXGQXGQ

SEQ ID NO: 439 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGEXGEXGEXGEXGEXGEXGE

SEQ ID NO: 440 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGQXGQXGQXGQXGQXGQXGQ

SEQ ID NO: 441 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGDXGDXGDXGDXGDXGDXGD

SEQ ID NO: 442 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGNXGNXGNXGNXGNXGNXGN

SEQ ID NO: 443 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGDXGDXGDXGDXGDXGDXGD

SEQ ID NO: 444 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGNXGNXGNXGNXGNXGNXGN

SEQ ID NO: 445 - protein, 21 aa, synthetic construct
  REGION 1..21: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XGPXGPXGPXGPXGPXGPXGP

SEQ ID NO: 446 - protein, 21 aa, synthetic construct
  REGION 1..21: note = Synthetic
  MOD_RES 1: note = Fluoroproline
  MOD_RES 4: note = Fluoroproline
  MOD_RES 7: note = Fluoroproline
  MOD_RES 10: note = Fluoroproline
  MOD_RES 13: note = Fluoroproline
  MOD_RES 16: note = Fluoroproline
  MOD_RES 19: note = Fluoroproline
XGPXGPXGPXGPXGPXGPXGP

SEQ ID NO: 447 - protein, 21 aa, synthetic construct
  REGION 1..21: note = Synthetic
  MOD_RES 3: note = 4(R)-hydroxyproline
  MOD_RES 6: note = 4(R)-hydroxyproline
  MOD_RES 9: note = 4(R)-hydroxyproline
  MOD_RES 12: note = 4(R)-hydroxyproline
  MOD_RES 15: note = 4(R)-hydroxyproline
  MOD_RES 18: note = 4(R)-hydroxyproline
  MOD_RES 21: note = 4(R)-hydroxyproline
PGXPGXPGXPGXPGXPGXPGX

SEQ ID NO: 448 - protein, 21 aa, synthetic construct
  REGION 1..21: note = Synthetic
  MOD_RES 1: note = Fluoroproline
  MOD_RES 3: note = 4(R)-hydroxyproline
  MOD_RES 4: note = Fluoroproline
  MOD_RES 6: note = 4(R)-hydroxyproline
  MOD_RES 7: note = Fluoroproline
  MOD_RES 9: note = 4(R)-hydroxyproline
  MOD_RES 10: note = Fluoroproline
  MOD_RES 12: note = 4(R)-hydroxyproline
  MOD_RES 13: note = Fluoroproline
  MOD_RES 15: note = 4(R)-hydroxyproline
  MOD_RES 16: note = Fluoroproline
  MOD_RES 18: note = 4(R)-hydroxyproline
  MOD_RES 19: note = Fluoroproline
  MOD_RES 21: note = 4(R)-hydroxyproline
XGXXGXXGXXGXXGXXGXXGX

SEQ ID NO: 449 - protein, 21 aa, synthetic construct
  REGION 1..21: note = Synthetic
  MOD_RES 1: note = 4(R)-hydroxyproline
  MOD_RES 3..4: note = 4(R)-hydroxyproline
  MOD_RES 6..7: note = 4(R)-hydroxyproline
  MOD_RES 9..10: note = 4(R)-hydroxyproline
  MOD_RES 12..13: note = 4(R)-hydroxyproline
  MOD_RES 15..16: note = 4(R)-hydroxyproline
  MOD_RES 18..19: note = 4(R)-hydroxyproline
  MOD_RES 21: note = 4(R)-hydroxyproline
XGXXGXXGXXGXXGXXGXXGX

SEQ ID NO: 450 - protein, 21 aa, synthetic construct
  REGION 1..21: note = Synthetic
  MOD_RES 1: note = 4(R)-hydroxyproline
  MOD_RES 3: note = Fluoroproline
  MOD_RES 4: note = 4(R)-hydroxyproline
  MOD_RES 6: note = Fluoroproline
  MOD_RES 7: note = 4(R)-hydroxyproline
  MOD_RES 9: note = Fluoroproline
  MOD_RES 10: note = 4(R)-hydroxyproline
  MOD_RES 12: note = Fluoroproline
  MOD_RES 13: note = 4(R)-hydroxyproline
  MOD_RES 15: note = Fluoroproline
  MOD_RES 16: note = 4(R)-hydroxyproline
  MOD_RES 18: note = Fluoroproline
  MOD_RES 19: note = 4(R)-hydroxyproline
  MOD_RES 21: note = Fluoroproline
XGXXGXXGXXGXXGXXGXXGX

SEQ ID NO: 451 - protein, 21 aa, synthetic construct
  REGION 1..21: note = Synthetic
  MOD_RES 3: note = Fluoroproline
  MOD_RES 6: note = Fluoroproline
  MOD_RES 9: note = Fluoroproline
  MOD_RES 12: note = Fluoroproline
  MOD_RES 15: note = Fluoroproline
  MOD_RES 18: note = Fluoroproline
  MOD_RES 21: note = Fluoroproline
PGXPGXPGXPGXPGXPGXPGX

SEQ ID NO: 452 - protein, 21 aa, synthetic construct
  REGION 1..21: note = Synthetic
  MOD_RES 1: note = Fluoroproline
  MOD_RES 3..4: note = Fluoroproline
  MOD_RES 6..7: note = Fluoroproline
  MOD_RES 9..10: note = Fluoroproline
  MOD_RES 12..13: note = Fluoroproline
  MOD_RES 15..16: note = Fluoroproline
  MOD_RES 18..19: note = Fluoroproline
  MOD_RES 21: note = Fluoroproline
XGXXGXXGXXGXXGXXGXXGX

SEQ ID NO: 453 - protein, 22 aa, synthetic construct
  REGION 1..22: note = Synthetic
CPPGPPGPPGPPGPPGPPGPPG

SEQ ID NO: 454 - protein, 22 aa, synthetic construct
  REGION 1..22: note = Synthetic
PPGPPGPPGPPGPPGPPGPPGC

SEQ ID NO: 455 - protein, 23 aa, synthetic construct
  REGION 1..23: note = Synthetic
CPPGPPGPPGPPGPPGPPGPPGC

SEQ ID NO: 456 - protein, 22 aa, synthetic construct
  REGION 1..22: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
CXXGXXGXXGXXGXXGXXGXXG

SEQ ID NO: 457 - protein, 22 aa, synthetic construct
  REGION 1..22: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGXXGXXGC

SEQ ID NO: 458 - protein, 23 aa, synthetic construct
  REGION 1..23: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
CXXGXXGXXGXXGXXGXXGXXGC

SEQ ID NO: 459 - protein, 22 aa, synthetic construct
  REGION 1..22: note = Synthetic
MPPGPPGPPGPPGPPGPPGPPG

SEQ ID NO: 460 - protein, 22 aa, synthetic construct
  REGION 1..22: note = Synthetic
PPGPPGPPGPPGPPGPPGPPGM

SEQ ID NO: 461 - protein, 23 aa, synthetic construct
  REGION 1..23: note = Synthetic
MPPGPPGPPGPPGPPGPPGPPGM

SEQ ID NO: 462 - protein, 22 aa, synthetic construct
  REGION 1..22: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
MXXGXXGXXGXXGXXGXXGXXG

SEQ ID NO: 463 - protein, 22 aa, synthetic construct
  REGION 1..22: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGXXGXXGM

SEQ ID NO: 464 - protein, 23 aa, synthetic construct
  REGION 1..23: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
MXXGXXGXXGXXGXXGXXGXXGM

SEQ ID NO: 465 - protein, 22 aa, synthetic construct
  REGION 1..22: note = Synthetic
KPPGPPGPPGPPGPPGPPGPPG

SEQ ID NO: 466 - protein, 22 aa, synthetic construct
  REGION 1..22: note = Synthetic
PPGPPGPPGPPGPPGPPGPPGK

SEQ ID NO: 467 - protein, 23 aa, synthetic construct
  REGION 1..23: note = Synthetic
KPPGPPGPPGPPGPPGPPGPPGK

SEQ ID NO: 468 - protein, 22 aa, synthetic construct
  REGION 1..22: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
KXXGXXGXXGXXGXXGXXGXXG

SEQ ID NO: 469 - protein, 22 aa, synthetic construct
  REGION 1..22: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGXXGXXGK

SEQ ID NO: 470 - protein, 23 aa, synthetic construct
  REGION 1..23: note = Synthetic
  MOD_RES 2: note = Fluoroproline
  MOD_RES 3: note = 4(R)-hydroxyproline
  MOD_RES 5: note = Fluoroproline
  MOD_RES 6: note = 4(R)-hydroxyproline
  MOD_RES 8: note = Fluoroproline
  MOD_RES 9: note = 4(R)-hydroxyproline
  MOD_RES 11: note = Fluoroproline
  MOD_RES 12: note = 4(R)-hydroxyproline
  MOD_RES 14: note = Fluoroproline
  MOD_RES 15: note = 4(R)-hydroxyproline
  MOD_RES 17: note = Fluoroproline
  MOD_RES 18: note = 4(R)-hydroxyproline
  MOD_RES 20: note = Fluoroproline
  MOD_RES 21: note = 4(R)-hydroxyproline
KXXGXXGXXGXXGXXGXXGXXGK

SEQ ID NO: 471 - protein, 10 aa, synthetic construct
  REGION 1..10: note = Synthetic
PKPQQFFGLM

SEQ ID NO: 472 - protein, 32 aa, synthetic construct
  REGION 1..32: note = Synthetic
PPGPPGPPGPPGPPGPPGPPGRPKPQQFFGLM

SEQ ID NO: 473 - protein, 32 aa, synthetic construct
  REGION 1..32: note = Synthetic
  MOD_RES 1: note = 4(R)-hydroxyproline
  MOD_RES 4: note = 4(R)-hydroxyproline
  MOD_RES 7: note = 4(R)-hydroxyproline
  MOD_RES 10: note = 4(R)-hydroxyproline
  MOD_RES 13: note = 4(R)-hydroxyproline
  MOD_RES 16: note = 4(R)-hydroxyproline
  MOD_RES 19: note = 4(R)-hydroxyproline
XPGXPGXPGXPGXPGXPGXPGRPKPQQFFGLM

SEQ ID NO: 474 - protein, 32 aa, synthetic construct
  REGION 1..32: note = Synthetic
  MOD_RES 2: note = 4(R)-hydroxyproline
  MOD_RES 5: note = 4(R)-hydroxyproline
  MOD_RES 8: note = 4(R)-hydroxyproline
  MOD_RES 11: note = 4(R)-hydroxyproline
  MOD_RES 14: note = 4(R)-hydroxyproline
  MOD_RES 17: note = 4(R)-hydroxyproline
  MOD_RES 20: note = 4(R)-hydroxyproline
PXGPXGPXGPXGPXGPXGPXGRPKPQQFFGLM

SEQ ID NO: 475 - protein, 32 aa, synthetic construct
  REGION 1..32: note = Synthetic
  MOD_RES 1: note = Fluoroproline
  MOD_RES 2: note = 4(R)-hydroxyproline
  MOD_RES 4: note = Fluoroproline
  MOD_RES 5: note = 4(R)-hydroxyproline
  MOD_RES 7: note = Fluoroproline
  MOD_RES 8: note = 4(R)-hydroxyproline
  MOD_RES 10: note = Fluoroproline
  MOD_RES 11: note = 4(R)-hydroxyproline
  MOD_RES 13: note = Fluoroproline
  MOD_RES 14: note = 4(R)-hydroxyproline
  MOD_RES 16: note = Fluoroproline
  MOD_RES 17: note = 4(R)-hydroxyproline
  MOD_RES 19: note = Fluoroproline
  MOD_RES 20: note = 4(R)-hydroxyproline
XXGXXGXXGXXGXXGXXGXXGRPKPQQFFGLM

SEQ ID NO: 476 - protein, 32 aa, synthetic construct; identical to SEQ ID NO: 475
  REGION 1..32: note = Synthetic
  MOD_RES 1: note = Chloroproline
  MOD_RES 2: note = 4(R)-hydroxyproline
  MOD_RES 4: note = Chloroproline
  MOD_RES 5: note = 4(R)-hydroxyproline
  MOD_RES 7: note = Chloroproline
  MOD_RES 8: note = 4(R)-hydroxyproline
  MOD_RES 10: note = Chloroproline
  MOD_RES 11: note = 4(R)-hydroxyproline
  MOD_RES 13: note = Chloroproline
  MOD_RES 14: note = 4(R)-hydroxyproline
  MOD_RES 16: note = Chloroproline
  MOD_RES 17: note = 4(R)-hydroxyproline
  MOD_RES 19: note = Chloroproline
  MOD_RES 20: note = 4(R)-hydroxyproline

SEQ ID NO: 477 - protein, 32 aa, synthetic construct; identical to SEQ ID NO: 474
  REGION 1..32: note = Synthetic
  MOD_RES 2: note = Fluoroproline
  MOD_RES 5: note = Fluoroproline
  MOD_RES 8: note = Fluoroproline
  MOD_RES 11: note = Fluoroproline
  MOD_RES 14: note = Fluoroproline
  MOD_RES 17: note = Fluoroproline
  MOD_RES 20: note = Fluoroproline

SEQ ID NO: 478 - protein, 32 aa, synthetic construct; identical to SEQ ID NO: 474
  REGION 1..32: note = Synthetic
  MOD_RES 2: note = Chloroproline
  MOD_RES 5: note = Chloroproline
  MOD_RES 8: note = Chloroproline
  MOD_RES 11: note = Chloroproline
  MOD_RES 14: note = Chloroproline
  MOD_RES 17: note = Chloroproline
  MOD_RES 20: note = Chloroproline

SEQ ID NO: 479 - protein, 30 aa, synthetic construct
  REGION 1..30: note = Synthetic
PPGPPGPPGPPGPPGPPGPPGPPGPPGPPG